(19) United States
(12) Patent Application Publication
(10) Pub. No.: US 2023/0355255 A1
(43) Pub. Date: Nov. 9, 2023

(54) METHODS AND DEVICES FOR TREATING VASCULAR DISEASE

(71) Applicant: Route 92 Medical, Inc., San Mateo, CA (US)

(72) Inventors: Tony M. Chou, San Mateo, CA (US); John Miller, San Mateo, CA (US); Joey English, San Mateo, CA (US); Warren T. Kim, San Mateo, CA (US); Michi E. Garrison, San Mateo, CA (US)

(21) Appl. No.: 18/311,797

(22) Filed: May 3, 2023

Related U.S. Application Data
(60) Provisional application No. 63/346,524, filed on May 27, 2022.
(60) Provisional application No. 63/338,114, filed on May 4, 2022.

(51) Int. Cl. A61B 17/22; A61F 2/966

(52) U.S. Cl. CPC A61B 17/22; A61F 2/966; A61B 2017/22038; A61B 2017/22079; A61B 2017/22094; A61M 2025/0004

(57) ABSTRACT

Methods of treating intracranial atherosclerotic disease carotid atherosclerosis, and intracranial and cerebral aneurysms by deploying implantable expandable devices. A catheter system is advanced through a base sheath towards an intracranial vessel having an atherosclerotic lesion. A tapered end region of an inner catheter is positioned distal to a distal end of an outer catheter, at least a portion of the tapered end region of the inner catheter crosses the lesion. The outer catheter is advanced over the inner catheter across the lesion. The inner catheter is withdrawn while the outer catheter is maintained in place. A stent delivery system is advanced through the catheter lumen. The outer catheter is withdrawn to unsleeve the stent and the stent delivery system maintained in place. The stent is deployed against the lesion. Related devices, systems, and methods are provided.

### CROSS-REFERENCE TO RELATED APPLICATIONS

This application claims the benefit of priority under 35 U.S.C. § 119(e) to U.S. Provisional Pat. Application Serial No. 63/338,114, filed May 4, 2022 and U.S. Provisional Pat. Application Serial No. 63/346,524, filed May 27, 2022. The disclosures are hereby incorporated by reference in their entireties.

### FIELD

The present technology relates generally to medical devices and methods, and more particularly, to medical device delivery and methods of implantation of stents or flow diverters for the treatment of vascular disease such as blood vessel narrowing due to vasospasm or atherosclerotic disease and intracranial aneurysm.

### BACKGROUND

Vascular disease caused by stenosis or narrowing of a vessel is commonly treated by endovascular implantation of scaffolding devices such as stents, often in combination with balloon angioplasty, to increase the inner diameter or cross-sectional area of the vessel lumen. Endovascular implantation of scaffolding devices such as stents or flow diverters can also be used to treat aneurysms to direct flow and/or assist in the implantation of a coil into the aneurysm.

Treating vessels of the brain (e.g. cerebral arteries) or vessels leading to the brain (e.g., carotid arteries) by endovascular implantation of stents and stent-like devices is particularly challenging due, in part, to the tortuosity of the vasculature in the skull and the small size of the vessels. Further, the risk of stroke and thromboembolic complications is high due to the release of thrombotic material during delivery of the stent and, in the case of flow diverters for treatment of aneurysm, can block blood flow to branch vessels. Stent length also poses a risk for further thromboembolic complications. In addition, navigating through the delicate intracranial cerebral vessels that are highly stenotic or fully blocked can be risky because the size and direction of the vessels are not well-visualized. Advancing balloons, stent delivery catheters, or other treatment devices through these vessels blindly or with limited flow increases risk of injury such as vessel dissection or perforation.

Guide catheters or guide sheaths are used to guide interventional devices to the target anatomy from an arterial access site, typically the femoral artery. The length of the guide is determined by the distance between the access site and the desired location of the guide distal tip. Interventional devices such as guidewires, microcatheters, and intermediate catheters used for sub-selective guides and aspiration, are inserted through the guide and advanced to the target site. Often, devices are used in a co-axial fashion, namely, a guidewire inside a microcatheter inside an intermediate catheter is advanced as an assembly to the target site in a stepwise fashion with the inner, most atraumatic elements, advancing distally first and providing support for advancement of the outer elements. The length of each element of the coaxial assemblage takes into account the length of the guide, the length of proximal connectors on the catheters, and the length needed to extend from the distal end.

Typical tri-axial systems such as for aspiration or delivery of stents, stent retrievers and other interventional devices require overlapped series of catheters, each with their own rotating hemostatic valves (RHV) on the proximal end. For example, a guidewire can be inserted through a Penumbra VELOCITY microcatheter having a first proximal RHV, which can be inserted through a Penumbra ACE68 having a second proximal RHV, which can be inserted through a Penumbra NEURONMAX 088 access catheter having a third proximal RHV positioned in the high carotid via a femoral introducer. Maintaining the coaxial relationships between these catheters can be technically challenging. The three RHVs must be constantly adjusted with two hands or, more commonly, four hands (i.e., two operators). Further, the working area of typical tri-axial systems for aspiration and/or intracranial device delivery can require working area of 3-5 feet at the base of the operating table.

The time required to access the site of the occlusion and restore, even partially, flow to the vessel is crucial in determining a successful outcome of such procedures. Similarly, the occurrence of distal emboli during the procedure and the potentially negative neurologic effect and procedural complications such as perforation and intracerebral hemorrhage are limits to success of the procedure. There is also difficulty in getting larger-bore access catheters and sheaths in a rapid and atraumatic fashion to distal carotid and cerebral vessels. Both the lengths and diameters of current systems put limitations on the delivery system of endovascular scaffolding devices such as stents, or flow diverters, which in turn limits the safety, speed, and precision of delivering such devices. There is a need for a system of devices and methods that allow for rapid access of distal carotid and cerebral vessels with larger lumen sizes and/or shorter lengths. There is also a need for improved delivery systems of scaffolding devices, and corresponding improved scaffolding device designs, which may be delivered through improved, larger lumen, and/or shorter system of access devices. There is also a need for improved navigation and access devices that can cross high-grade or fully stenosed vessels with minimal risk of trauma to aid in delivery of devices such as balloon catheters and stents.

In an interrelated aspect, provided is a flow diverter system including a delivery system having an inner tubular member; and an outer tubular member. The flow diverter system includes a flow diverter mounted on the inner tubular member and constrained by the outer tubular member during delivery; and an outer catheter having an inner diameter of between 2.0 mm and 3.0 mm configured to receive the flow diverter constrained by the outer tubular member for delivery.

The flow diverter can be a laser-cut expandable metal tube. The flow diverter can be formed of first and second expandable tubes. The first and second expandable tubes can each be a laser-cut metal tube. The first expandable tube can be a laser-cut metal tube and the second expandable tube can be a braided tube. The first expandable tube can be a laser-cut metal tube and the second expandable tube can be a polymer sleeve. The flow diverter can have a compound construction. The compound construction can include two end sections constructed from laser-cut tube and a middle section having a braid.

In an interrelated aspect, provided is a flow diverter system including a flow diverter delivery system having an inner tubular member and an introducer; and a flow diverter mounted on the inner tubular member and constrained by the introducer. The flow diverter constrained by the introducer is deliverable through a delivery catheter having an inner diameter of between 2.0 mm and 3.0 mm.

### SUMMARY

In an aspect, disclosed is a method of treating intracranial atherosclerotic disease. The method includes advancing a catheter system through a base sheath towards an intracranial vessel having an atherosclerotic lesion. The catheter system includes an inner catheter having a tubular elongate body with a single lumen and a flexible, distal tapered end region; and an outer catheter having a catheter lumen and a distal end. The method includes positioning the tapered end region of the inner catheter distal to the distal end of the outer catheter; crossing the lesion with at least a portion of the tapered end region of the inner catheter; advancing the outer catheter over the inner catheter and positioning a distal end region of the outer catheter across the lesion; withdrawing the inner catheter from the catheter lumen and maintaining the outer catheter in place across the lesion; advancing a stent delivery system having a stent through the catheter lumen to the distal end region of the outer catheter; withdrawing the outer catheter to unsleeve the stent and maintaining the stent delivery system in place; and deploying the stent of the stent delivery system against the lesion.

The method can further include navigating the catheter system through a carotid artery using the tapered end region of the inner catheter to find a passage through an occlusion in the carotid artery. Crossing the lesion with the at least a portion of the tapered end region of the inner catheter can pre-dilate the lesion. Advancing the catheter system includes advancing the catheter system over a guidewire. The guidewire can be pre-positioned across the lesion. The guidewire can be positioned within the single lumen of the inner catheter proximal to a distal opening from the single lumen. Advancing a catheter system through a base sheath can further include navigating the catheter system through a carotid artery while the tapered end region of the inner catheter is positioned distal to the distal end of the outer catheter and the guidewire is fully contained within the single lumen of the inner catheter. Navigating the catheter system through the carotid artery can include using the tapered end region of the inner catheter to find a passage through an occlusion in the carotid artery.

The tapered end region of the inner catheter can dilate the occlusion in the carotid artery as the catheter system is advanced towards the atherosclerotic lesion in the intracranial vessel. A distal end of the guidewire can be positioned proximal to the distal tapered end region of the inner catheter during the advancing step. The guidewire can be a 0.014” to 0.024” guidewire. The inner catheter can have a length configured to extend from outside a patient’s body, through a femoral artery, and to the intracranial vessel. The inner catheter can further include a proximal segment having a metal reinforced segment and an intermediate segment having an unreinforced polymer having a first durometer, the intermediate segment proximal of the distal tapered end region and distal to the proximal segment. The distal tapered end region can be formed of a polymer that is different from the unreinforced polymer of the intermediate segment, and where the polymer of the tapered end region has a second durometer less than the first durometer. The tapered end region can taper distally from a first outer diameter of between 0.048” and 0.080” to a second outer diameter of about 0.031” up to about 0.048” over a length that is between 0.5 cm and 4.0 cm. The second outer diameter can be at a distal-most terminus of the inner catheter. A taper angle of a wall of the tapered end region relative to a center line of the tapered end region can be between 0.9 to 1.6 degrees. The second outer diameter can be about 50% of the first outer diameter, about 40% of the first outer diameter, or about 65% of the first outer diameter. The intermediate segment can include a first segment having a material hardness of no more than 55 D and a second segment located proximal to the first segment having a material hardness of no more than 72 D. A location of a material transition between the unreinforced polymer and the metal reinforced segment can be at least about 49 cm from a distal end of the elongate body.

In an interrelated aspect, provided is a method of treating intracranial atherosclerotic disease including advancing a catheter system through a base sheath towards an intracranial vessel having an atherosclerotic lesion. The catheter system includes an inner catheter having a tubular elongate body with a single lumen and a flexible, distal tapered end region; and an outer catheter having a catheter lumen and a distal end. The method includes positioning the tapered end region of the inner catheter distal to the distal end of the outer catheter; crossing the lesion with at least a portion of the tapered end region of the inner catheter to pre-dilate the lesion; positioning a distal end of the outer catheter to a proximal base of the lesion; withdrawing the inner catheter from the catheter lumen and maintaining the outer catheter in place; advancing a stent delivery system having a stent through the catheter lumen through the distal end of the outer catheter and into the pre-dilated lesion; and deploying the stent of the stent delivery system against the lesion.

In an interrelated aspect, provided is a method of treating atherosclerotic disease including advancing a distal end of a base sheath from a femoral artery to a common carotid artery; advancing a catheter system through the base sheath towards an atherosclerotic lesion in at least one of a common carotid artery, an external carotid artery, or an internal carotid artery. The catheter system includes an inner catheter having a tubular elongate body with a single lumen and a flexible, distal tapered end region; and an outer catheter. The outer catheter includes a flexible, distal luminal portion having a catheter lumen extending between a distal end and a proximal end of the flexible, distal luminal portion; and a proximal tether element extending proximally from a point of attachment near the proximal end of the flexible distal luminal portion to outside the body of the patient. An outer diameter of a portion of the proximal tether element near the point of attachment is smaller than an outer diameter of the distal luminal portion near the point of attachment. The method includes positioning the tapered end region of the inner catheter distal to the distal end of the outer catheter; crossing the lesion with at least a portion of the tapered end region of the inner catheter; withdrawing the inner catheter from the catheter lumen and maintaining the outer catheter in place; advancing a stent delivery system having a stent through the catheter lumen to the distal end region of the outer catheter; and deploying the stent of the stent delivery system against the lesion.

Crossing the lesion with the at least a portion of the tapered end region of the inner catheter can dilate the lesion. Advancing the catheter system includes advancing the catheter system with a guidewire positioned within the single lumen of the inner catheter so a distal end of the guidewire is positioned proximal to a distal opening from the single lumen. Crossing the lesion can include navigating the catheter system past the lesion while the tapered end region of the inner catheter is positioned distal to the distal end of the outer catheter and without the guidewire extending out of the distal opening of the single lumen of the inner catheter. Navigating the catheter system past the lesion can include using the tapered end region of the inner catheter to find a passage through the lesion. The distal end of the base sheath can be advanced to a location proximal of a bifurcation between the internal carotid artery and the external carotid artery. The method can further include advancing the outer catheter over the inner catheter and positioning a distal end region of the outer catheter across the lesion. The method can further include withdrawing the outer catheter after advancing the stent delivery system to unsleeve the stent while maintaining the stent delivery system in place.

In an interrelated aspect, provided is a method of treating atherosclerotic disease including advancing a distal end of a base sheath from a femoral artery to a common carotid artery; advancing a catheter system through the base sheath towards an atherosclerotic lesion in at least one of a common carotid artery, an external carotid artery, or an internal carotid artery. The catheter system includes an inner catheter having a tubular elongate body with a single lumen. The inner catheter includes a proximal segment, an intermediate segment, and a flexible, distal tapered end region having an unreinforced polymer with a material hardness less than that of the intermediate segment, a taper length of the tapered end region being between about 0.5 cm and about 4.0 cm. The catheter system includes an outer catheter having a catheter lumen extending between a distal end and a proximal end. The method includes positioning the tapered end region of the inner catheter distal to the distal end of the outer catheter; crossing the lesion with at least a portion of the tapered end region of the inner catheter; withdrawing the inner catheter from the catheter lumen and maintaining the outer catheter in place; advancing a stent delivery system having a stent through the catheter lumen to the distal end region of the outer catheter; and deploying the stent of the stent delivery system against the lesion.

Crossing the lesion with the at least a portion of the tapered end region of the inner catheter can dilate the lesion. Advancing the catheter system can include advancing the catheter system with a guidewire positioned within the single lumen of the inner catheter so a distal end of the guidewire is positioned proximal to a distal opening from the single lumen. Crossing the lesion can include navigating the catheter system past the lesion while the tapered end region of the inner catheter is positioned distal to the distal end of the outer catheter and without the guidewire extending out of the distal opening of the single lumen of the inner catheter. Navigating the catheter system past the lesion can include using the tapered end region of the inner catheter to find a passage through the lesion. The distal end of the base sheath can be advanced to a location proximal of a bifurcation between the internal carotid artery and the external carotid artery. The method can further include advancing the outer catheter over the inner catheter and positioning a distal end region of the outer catheter across the lesion. The method can further include withdrawing the outer catheter after advancing the stent delivery system to unsleeve the stent while maintaining the stent delivery system in place.

### BRIEF DESCRIPTION OF THE DRAWINGS

These and other aspects will now be described in detail with reference to the following drawings. Generally, the figures are not to scale in absolute terms or comparatively, but are intended to be illustrative. Also, relative placement of features and elements may be modified for the purpose of illustrative clarity.

FIG. 1A shows a catheter system for accessing an occlusion site in an artery;

FIG. 1B shows the catheter system of FIG. 1A assembled;

FIG. 1C is a detail view of a distal end region of a catheter advancement element taken along circle C-C of FIG. 1A;

FIG. 1D is a detail view of a distal end region of a catheter advancing element having a rescue guidewire parked proximal of the distal opening;

FIG. 2A shows a schematic of a conventional guidewire centered by a microcatheter and penetrating an occlusion such as an embolus or atherosclerotic lesion;

FIG. 2B shows a schematic of a catheter advancement element positioned within a vessel and the tapered distal tip region deflecting upon reaching a proximal face of an occlusion;

FIG. 2C shows a schematic of a catheter advancement element positioned over a guidewire within a vessel having a tapered distal tip region deflecting upon reaching a proximal face of an occlusion;

FIG. 3A shows an implementation of a test rig for assessing deflection of a tapered distal tip region upon reaching a proximal face of an occlusion such as an embolus or atherosclerotic lesion;

FIG. 3B is a schematic of an implementation of a test rig;

FIG. 4A shows an assembled catheter system being advanced through a base sheath positioned in the internal carotid artery (ICA) towards an atherosclerotic lesion in an intracranial vessel;

FIGS. 4B-4D show the tapered distal end region of the inner catheter crossing the lesion to pre-dilate the lesion;

FIG. 4E shows the outer catheter advanced over the inner catheter and positioning a distal end region of the outer catheter across the lesion;

FIG. 4F shows the inner catheter withdrawn from the outer catheter while the outer catheter is maintained in place across the lesion;

FIG. 4G shows a stent delivery system advanced through the outer catheter to the distal end region of the outer catheter;

FIG. 4H shows the outer catheter withdrawn relative to the stent delivery system and the stent delivery system maintained in place;

FIG. 4I shows the stent deployed against the lesion and the stent delivery system withdrawn;

FIG. 5A shows the tapered distal end region of the inner catheter crossing the lesion to pre-dilate the lesion and the distal end of the outer catheter positioned at the proximal base of the lesion;

FIG. 5B shows the inner catheter withdrawn from the outer catheter while the outer catheter is maintained in place at the proximal base of the lesion;

FIG. 5C shows a stent delivery system advanced through the outer catheter into the pre-dilated lesion;

FIG. 5D shows the stent of the stent delivery system deployed against the lesion;

FIG. 6A is an implementation of a cut-tube flow diverter in a collapsed delivery configuration;

FIG. 6B is the flow diverter of FIG. 6A in the expanded configuration;

FIGS. 7A-7C show details of an attachment mechanism between two layers of a dual-layer flow diverter;

FIGS. 8A-8B show details of an alternate attachment mechanism between two layers of a dual-layer flow diverter;

FIGS. 9A-9B show embodiments of a flow diverter;

FIG. 10 shows an embodiment of a compound flow diverter;

FIG. 11A shows an assembled catheter system accessing an intracranial aneurysm, with a base sheath positioned in the internal carotid artery (ICA), an outer catheter advanced in the distal ICA, and an inner catheter crossing the vessel in the area of the aneurysm;

FIG. 11B shows the outer catheter of FIG. 11A advanced across the aneurysm and the inner catheter withdrawn;

FIG. 11C shows show a delivery system advanced across the aneurysm and the outer catheter withdrawn;

FIG. 11D shows the restraining sleeve of the delivery system withdrawn and an endovascular scaffolding device deployed across the aneurysm;

FIG. 12A is a side view of an implementation of a catheter advancement element having a rapid-exchange guidewire lumen;

FIG. 12B is a cross-sectional view of the catheter advancement element of FIG. 12A;

FIG. 13A shows components of a delivery system for an endovascular scaffolding device such as a flow diverter or stent;

FIG. 13B shows the device and delivery system of FIG. 13A assembled in a delivery configuration;

FIG. 13C shows the device and delivery system of FIG. 13B with the device partially deployed.

It should be appreciated that the drawings are for example only and are not meant to be to scale. It is to be understood that devices described herein may include features not necessarily depicted in each figure.

### DETAILED DESCRIPTION

There is a need for devices, systems, and methods to safely and quickly access and treat occlusions or aneurysms within the cerebral arteries. In particular, there is a need for access systems to deliver balloon angioplasty and/or endovascular scaffolding devices, such as stents or flow diverters, to vessels in the brain so that occlusions or aneurysms may be treated quickly, accurately, and safely by using access methods that are potentially either or both shorter in length and larger in diameter.

In addition, there is a need for improved endovascular scaffolding devices and device delivery systems, which are enabled by larger and/or shorter access systems.

Acute ischemic stroke (AIS) is the sudden blockage of adequate arterial blood flow to a section of the brain, usually caused by emboli lodging or thrombus forming in situ in one of the blood vessels supplying the brain. If this blockage is not quickly resolved, ischemia may lead to permanent neurologic deficit or death. The timeframe for effective treatment of stroke is within 3-4 hours for intravenous (IV) thrombolytic therapy and 6 hours for site-directed intra-arterial thrombolytic therapy or up to 7-8 hours for interventional recanalization of a blocked cerebral artery. Re-perfusing the ischemic brain after this time period has no overall benefit to the patient, and may in fact cause harm due to the increased risk of intracranial hemorrhage from fibrinolytic use. Even within this time period, there is strong evidence that the shorter the time period between onset of symptoms and treatment, the better the results. Unfortunately, the ability to recognize symptoms, deliver patients to stroke treatment sites, and finally to treat these patients within this timeframe is rare. Despite treatment advances, stroke remains the third leading cause of death and the leading cause of serious, long-term disability in the United States.

Atherosclerosis is the build-up of fatty deposits and/or plaque on the inner wall of a patient’s arteries. The lesions decrease the size of the artery lumen and limit blood flow through the artery. Intracranial vessels including basilar artery, internal carotid arteries, middle cerebral arteries, intracranial vertebral arteries, posterior cerebral arteries, and anterior cerebral arteries can be prone to develop atherosclerosis including intimal necrosis, the accumulation of lipids and development of fatty streaks and fibromuscular plaque, intimal thickening and proliferative changes of the basement membrane and adventitia. Atherosclerotic disease in these vessels is re referred to as intracranial atherosclerotic disease (ICAD) and is considered a major cause of recurrent stroke and transient ischemic attacks. Stroke associated with ICAD occur most commonly due to artery-to-artery embolism as well as local branch occlusion, in situ thrombotic occlusion, and hemodynamic insufficiency (See Wondwossen et al. Neurology Vol. 97(20 S2): S 146-S 157 (2021). Unlike in AIS where the preferred treatment is removal of the clot, whether mechanically or by thrombolytic therapy, patients with ICAD stenosis may be treated with stenting to open the occluded artery resolving the hemodynamic insufficiency, preventing further narrowing and also mitigating the risk of artery-to-artery embolism and stroke associated with ICAD.

Navigating the arterial anatomy in order to treat various vascular pathologies at the level of the carotid arteries or cerebral arteries, requires catheter systems having superior flexibility and deliverability, which can be challenging for large-bore catheters. The internal carotid artery (ICA) arises from the bifurcation of the common carotid artery (CCA) at the level of the intervertebral disc between C3 and C4 vertebrae. The course of the ICA is divided into four parts - cervical Cr, petrous Pt, cavernous Cv and cerebral Cb parts. In the anterior circulation, the consistent tortuous terminal carotid is locked into its position by bony elements. The cervical carotid Cr enters the petrous bone and is locked into a set of turns as it is encased in bone. The cavernous carotid is an artery that passes through a venous bed, the cavernous sinus, and while flexible, is locked as it exits the cavernous sinus by another bony element, which surrounds and fixes the entry into the cranial cavity. Because of these bony points of fixation, the petrous and cavernous carotid (Pt and Cv) and above are relatively consistent in their tortuosity. The carotid siphon CS is an S-shaped part of the terminal ICA. The carotid siphon CS begins at the posterior bend of the cavernous ICA and ends at the ICA bifurcation into the anterior cerebral artery ACA and middle cerebral artery MCA. The ophthalmic artery arises from the cerebral ICA, which represents a common point of catheter hang up in accessing the anterior circulation. The MCA is initially defined by a single M1 segment and then further bifurcates in two or three M2 segments and then further arborizes to create M3 segments. These points of catheter hang up can significantly increase the amount of time needed to restore blood perfusion to the brain, which in the treatment of ICAD or AIS is a disadvantage with severe consequences.

In addition to the natural anatomy being problematic for navigating large-bore catheters to distal sites, the vessels leading to the distal sites can have one or more pathologies that are problematic for advancement of conventional guidewire-led systems. Patients may have an occlusion at a site proximal to the distal occlusion, such as within the carotid artery, that requires treatment before the catheter system can be navigated further. Patients may also have a severe atherosclerotic occlusion from a proximal location such as the cervical carotid that can extend through the origin of the ICA as high as the M1 segment. In this situation, the entire region is severely or completed occluded and is often poorly visualized or invisible on angiogram. A severely atherosclerotic vessel may also have a dissection or a breakdown of the layers of the vessel wall. Meaning, a flap of the vessel wall may protrude into the lumen. Partial or complete occlusions of the carotid and presence of arterial dissections greatly increases the difficulty in locating and navigating large-bore catheters through the anatomy. Conventional guidewire systems tend to go under a dissection flap causing the guidewire to get redirected away from the true lumen of the vessel and into branch vessels as well as greatly increasing the risk of vessel perforation.

With advancing age, the large vessels often enlarge and lengthen. Fixed proximally and distally, the cervical internal carotid artery often becomes tortuous with age. The common carotid artery CCA is relatively fixed in the thoracic cavity as it exits into the cervical area by the clavicle. The external and internal carotid arteries ECA, ICA are not fixed relative to the common carotid artery CCA, and thus they develop tortuosity with advancing age with lengthening of the entire carotid system. This can cause them to elongate and develop kinks and tortuosity or, in worst case, a complete loop or so-called “cervical loop”. If catheters used to cross these kinked or curved areas are too stiff or inflexible, these areas can undergo a straightening that can cause the vessel to wrap around or “barbershop pole” causing focused kinking and folding of the vessel. These sorts of extreme tortuosity also can significantly increase the amount of time needed to restore blood perfusion to the brain, particularly in the aging population. In certain circumstances, the twisting of vessels upon themselves or if the untwisted artery is kinked, normal antegrade flow may be reduced to a standstill creating ischemia. Managing the unkinking or unlooping the vessels such as the cervical ICA can also increase the time it takes to perform a procedure.

A major drawback of current catheter systems and methods for carotid and cerebral intervention procedures, such as the treatment of atherosclerosis with carotid stenting and/or intracranial stenting, is the amount of time required to restore blood perfusion to the brain, including the time it takes to access the occlusive site or sites in the cerebral artery. Reducing the time required to access the occlusion and exchange devices is an important factor in minimizing the overall time to a particular procedure. Additionally, each attempt is associated with potential procedural risk due to device advancement in the delicate cerebral vasculature.

Another limitation of current catheter systems typically used for stent delivery is the need for multiple operators to deliver and effectively manipulate long tri-axial systems with multiple RHVs typically used with conventional guide and distal access catheters. Tri-axial systems also impact the overall length requirements for working device delivery systems capable of navigation to distal sites. As an example, stent delivery systems are manufactured to have an effective length for delivery through a base sheath to a desired target location. The effective length of typical stent delivery systems for use in coronary vessels is between 140 cm and 150 cm and can be designed as over-the-wire or monorail delivery systems. Tri-axial access systems can limit the distal reach of such stent delivery system due to the additional length outside the patient that the stent delivery system must navigate. In other words, the stent delivery system used for coronary vessels can be too short to navigate to certain distal sites in the cerebral vasculature due to the inefficiency of the tri-axial point of access. Stent delivery systems are manufactured and stocked so that a variety of lengths and diameters of stents are available for a particular procedure with a standard catheter length. To accommodate the additional length of the tri-axial access longer stent delivery system may be used. The stent delivery system must be long enough to fit through the tri-axial system while still capable of reaching the target site. The stent itself must also be manufactured and stocked according to a variety of lengths and diameters.

There are a variety of stents known in the art including coronary stents, stents for peripheral artery disease affecting the femoral, popliteal and iliac arteries, as well as carotid and neurovascular stents. The two basic types of stents include balloon-expandable or self-expanding. One of the original balloon-expandable stents for use in the coronaries is the Palmaz-Schatz stent (J&J) and the WALLSTENT (Boston Scientific) was one of the first self-expanding stents for coronary use. Balloon-expandable stents tend to have greater radial outward force compared to self-expanding stents, but suffer from the susceptibility of compression by external mechanical forces. Self-expanding stents, in contrast, are able to return to their original shape even after compression by an external pressure. This characteristic is particularly important in peripheral stenting such as within the carotid artery or in the limbs.

The differences in material, construction and design of the stent (e.g., cross members, strut thickness, overall length, flexibility, rigidity) provides the stent with a functional property to perform at the desired treatment location. For example, a lesion that is prone to breaking off embolic material may need better coverage such as with a closed cell stent design. Lesions in certain anatomies such as the carotid area tend to be longer than lesions in the coronaries and benefit from a longer stent to ensure full coverage. Some stents like the WALLSTENT undergo a great deal of foreshortening upon expansion. Meaning, these otherwise long stents end up being even longer in the collapsed state to ensure full coverage of a lesion following foreshortening. Still further, a stent used in some anatomical locations may need greater radial strength to prevent strut recoil and the loss of lumen size such as within the limbs. Cell design, scaffolding, stent foreshortening, and outward radial force, etc. can each impact the deliverability of the stent through the anatomy to reach the lesion to be treated. Closed cell stents and stents that provide greater radial strength tend to be more rigid and less flexible. Longer, stiffer stents are more challenging to deliver to vessels, particularly where the path is highly tortuous, the size of the vessel is small, and the presence of disease along the way (e.g., extracranial carotid artery). The WALLSTENT, PRECISE Stent (Cordis), ZILVER (Cook), and others can be about 6 mm up to about 10 mm in diameter and are delivered through delivery catheters that have an inner diameter of about 0.070” up to about 0.088”. Large-bore access systems are beneficial in delivering relatively large neurovascular implants and devices such as stents and stent delivery systems. Typically, larger catheters, particularly catheters carrying relatively large stents, can be limited in their ability to reach distal sites due to lack of navigability and also limited length. As mentioned above, the path to reach distal sites undergoes tight turns locked within bony anatomy.

Neurovascular stents such as the NEUROFORM ATLAS (Stryker Neurovascular) or ENTERPRISE stent (Johnson & Johnson) have long delivery systems sufficient to reach distal sites, however are designed to support coil embolization and often lack radial force to effectively treat intracranial stenoses.

Navigation can be further complicated by atherosclerotic anatomy. Conventional catheter systems incorporating, for example, guidewires extending through microcatheters, tend to find undesirable pathways through atherosclerotic anatomy (e.g., under dissection flaps) creating delivery challenges and risks.

Guide catheters and guide sheaths are used to direct interventional devices, such as stents, coils, and flow diverters, to a target site, such as an embolism, stenosis, or an intracranial aneurysm, from the access site. It can be challenging to establish guide or sheath position in a fashion that is stable and provides support for the device delivery. To maneuver the catheters into position, coaxial, triaxial, or quadraxial systems are often used in which a guidewire/microcatheter system is first deployed and coaxial larger catheters are subsequently delivered. The clinical challenge, especially in the octogenarian population, is the elongation of the aortic arch against the fixed thoracic descending aorta, leading to a shifting of all great vessels, especially the brachiocephalic takeoff. Such shifting makes it more challenging to access the anatomy during treatment of, e.g., atherosclerosis, stroke, aneurysm, and other distally located vascular diseases. As catheters, wires, balloons, stents, or retrievable structures are advanced through the great vessels, they have a tendency to prolapse into the ascending aorta when pushed into a highly angulated and/or tortuous anatomy.

Additionally, due to difficulty in navigating large-diameter delivery systems to distal carotid and cerebral anatomies, devices such as flow diverters have been typically delivered through microcatheters that are 0.027” ID or smaller. Flow diverters are endoscaffolding devices used to treat unruptured aneurysms, especially aneurysms with wide necks that are difficult to exclude by other means such as embolic coils. Flow diverters are implanted in a segment of distal carotid or intracranial artery that includes the aneurysm. Flow diverters have a very dense material coverage, around 30% when expanded, so as to exclude or limit blood flow from entering the aneurysm through the aneurysm neck. Excluding blood flow into the aneurysm reduces or eliminates the risk of aneurysm rupture due to thrombosis at the site over time.

All currently available flow diverters are based on a braided wire design to achieve the high percentage metal coverage that achieves the desired thrombotic effect. A braid is the only design that can expand from a diameter deliverable through a 0.027” microcatheter to a maximum desired vessel diameter of up to 5 mm while still possessing a metal coverage ratio of 30% at the expanded configuration. Examples include the Medtronic PIPELINE, the Stryker SURPASS, the Terumo FRED, and others. In contrast, stents constructed from laser-cut metal tubes such as Nitinol, stainless steel, and other alloys are unable to accomplish this metal coverage ratio due to geometric constraints. The braid-style self-expanding implants may not immediately expand fully to the walls and may move during deployment, leading to time-consuming and risky maneuvers to achieve the desired wall coverage and wall apposition. Significant shortening of the braided flow diverters occurs during deployment due to the nature of braid construction, and often leads to ineffective coverage of the aneurysm site and often requires repositioning, manipulation, or may require placement of an additional implant. Because of this, coverage of the aneurysm and/or apposition of the flow diverter against the wall is often not optimal. Poor apposition is associated with higher rates of narrowing or occlusion of the flow diverter.

Additionally, due to difficulty in navigating large-diameter delivery systems to distal carotid and cerebral anatomies, devices such as flow diverters have been typically delivered through microcatheters that are 0.027” ID or smaller. Unfortunately, braided-style flow diverters can be difficult, time-consuming, imprecise, and risky to deliver. The delivery system for such devices often includes a leading distal guidewire tip, which presents risk of vessel perforation. The nature of braid-style implants often requires a delivery system with additional distal-end-constraining features, requiring multiple steps to deploy. The braid-style self-expanding implants may move and/or shorten during delivery and not immediately expand fully to the walls, leading to time-consuming and risky maneuvers to achieve the desired wall coverage and wall apposition. Even with these maneuvers, coverage and/or apposition is often not optimal.

Described herein are catheter systems and methods for treating various neurovascular pathologies, such as intracranial atherosclerotic disease (ICAD), lesions calcified with severe stenosis or a restenotic lesion by deploying a stent. The catheter systems described herein can also be used for treating and safely navigating through extracranial atherosclerotic disease to deploy a stent or flow diverter despite the visualization and navigational challenges. The systems described herein provide quick and simple single-operator access to distal target anatomy, in particular occluded anatomy of extracranial carotid arteries and the tortuous anatomy of the intracranial vasculature at a single point of manipulation. The medical methods, devices and systems described herein allow for navigating complex, tortuous anatomy to perform rapid and safe delivery of intracranial medical devices including stents, with or without aspiration for the treatment and/or removal of cerebral occlusions. The systems described herein can be particularly useful for the delivery of working devices in the treatment of atherosclerotic disease, including an angioplasty balloons, stents, or other working device, alone or in combination with aspiration. The catheter systems described herein can also be used to deliver endovascular scaffolding devices such as flow diverters to treat aneurysms by occluding flow to the aneurysm or to assist in the implantation of a coil into the aneurysm.

The devices, systems, and methods described herein allow the user to safely navigate and optimally place treatment systems with respect to an occlusion of vessel despite navigational challenges. The devices, systems, and methods provide a safer way to navigate occluded vessel and find the lumen. The devices, systems, and methods enable safe and rapid positioning of large interventional devices such as a large-bore aspiration catheters or stent delivery catheters to an occlusion in carotid or cerebral artery. Further, the extreme flexibility and deliverability of the distal access catheter systems described herein allow the catheters to take the shape of the tortuous anatomy rather than exert straightening forces creating new anatomy. The distal access catheter systems described herein can pass through tortuous loops while maintaining the natural curves of the anatomy therein decreasing the risk of vessel straightening. The distal access catheter systems described herein can thereby create a safe conduit through the neurovasculature maintaining the natural tortuosity of the anatomy for other catheters to traverse (e.g. interventional device delivery catheters). The catheters traversing the conduit need not have the same degree of flexibility and deliverability such that if they were delivered directly to the same anatomy rather than through the conduit, would lead to straightening, kinking, or folding of the anterior circulation.

It has been found in performing the novel methods described herein that a novel structure is desirable to extend the range of applications of a conventional catheter to these novel treatment approaches. Provided herein are systems including a catheter advancement element having a tapered distal end region with a flexibility, shape, and taper length configured to be atraumatically delivered to a vessel in the brain. This is not achieved with conventional catheter systems as they may have improper flexibility, are formed of improper materials, or have improper shape and/or taper length resulting in conventional catheter systems getting misdirected or hung up or, if more force is applied, perforating the vessel. Unlike these conventional catheter systems, the catheter systems described herein includes a catheter advancement element capable of safely navigating neurovascular anatomy and find the lumen so that a corresponding large bore catheter (i.e., aspiration and/or stent delivery system) can be delivered to distal sites. The catheter systems described herein help locate occlusions in the vessels in the novel manner of the methods provided herein. These and other features will be described in detail herein.

The systems described herein can be used for the delivery of a working device, such as a stent, for treatment of a carotid occlusion or atherosclerotic lesion, cerebral occlusion, ICAD lesion, aneurysm, or other pathology. The working device delivered can have its own delivery device, which can be advanced over a guidewire. The working device can be configured to provide thrombotic treatments and can include large-bore catheters, aspiration embolectomy (or thrombectomy), advanced catheters, wires, balloons. Preferably, the working device is an implantable structure - temporary and retrievable or an implant that remains in place following a procedure. The working device can be a retrievable structure such as coil-tipped retrievable stents “Stentriever” (e.g., SOLITAIRE by Medtronic or TREVO by Stryker) as well as permanent structures including flow diverters, and vessel support implants including balloon-expandable stents, self-expanding stents, and mesh sleeves. The working device can be a stent retriever having an expanding portion and a proximal control element. The working device can be a stent. As used herein, the term “stent” refers to a working device that is designed for use within a bodily structure such as within a body lumen and that is capable of undergoing a shape change from a lower profile insertion configuration to a higher profile deployed configuration. A stent refers to both balloon-expandable and self-expanding stents. A stent may be uncovered or covered with a material such as with a mesh, fabric sleeve, or graft material. A stent may be coated with a material such as a polymer or one or more drugs. A stent refers to an implant that remains in place within the bodily structure for a period of time following a procedure to continue providing a therapeutic effect. A stent may be permanent such as a metal stent or semi-permanent such as a bioabsorbable stent that erodes or is absorbed in a given time-frame. The stent may be a braided design, a cut metal tube design, or a multi-layer or compound design with more than one expandable element such as a braid and/or cut tube elements coupled together to form a single implant device.

The treatment system can also include a delivery device for delivering the working device. The delivery device can vary in structure and function depending upon the type of working device being deployed. In an implementation, the working device is a stent (e.g., self-expanding stent, braided stent, compound stent, or flow diverter) and the delivery device is a stent delivery system having an inner catheter on which the stent is mounted with an outer sheath configured to contain the stent against the inner catheter. The inner catheter can be a balloon catheter configured to post-dilate a self-expanding stent or expand the balloon expandable stent. The delivery device can include a catheter having a cylindrical region configured to support a working device such as a stent. The delivery device catheter can include a tapered distal end region extending distal to the cylindrical region supporting the working device. In an implementation, the working device is a stent or flow diverter and the delivery device is a microcatheter configured to house the expandable device. In conventional stent delivery systems, a microcatheter is positioned over a guidewire across the treatment site. The guidewire is then removed. The stent mounted on a delivery stylet is introduced into the proximal end of the microcatheter and pushed through the microcatheter via the stylet to the distal end of the microcatheter. The microcatheter is pulled back to deploy the stent. In still further implementations, the treatment system has no implantable working device and the delivery device is an angioplasty balloon catheter configured to dilate a target lesion. The balloon catheter can also have a tapered distal end region extending distal to the angioplasty balloon that is configured to pre-dilate the lesion prior to positioning the angioplasty balloon across the lesion.

While some implementations are described herein with specific regard to accessing a neurovascular anatomy or delivery of an expandable cerebral treatment device, the systems and methods described herein should not be limited to this and may also be applicable to other uses. For example, the catheter systems described herein may be used to deliver working devices to an extracranial vessel including the carotid vessels leading to the cerebral anatomy, or a target vessel of a coronary anatomy, peripheral anatomy, or other vasculature anatomy. Coronary vessels are considered herein including left and right coronary arteries, posterior descending artery, right marginal artery, left anterior descending artery, left circumflex artery, M1 and M2 left marginal arteries, and D1 and D2 diagonal branches. Any of a variety of peripheral vessels are considered herein including the popliteal arteries, anterior tibial arteries, dorsalis pedis artery, posterior tibial arteries, and fibular artery.

It should also be appreciated that where the phrase “aspiration catheter” is used herein that such a catheter may be used for other purposes besides or in addition to aspiration, such as the delivery of fluids to a treatment site or as a support catheter or distal access catheter providing a conduit that facilitates and guides the delivery or exchange of other devices such as a guidewire or interventional devices such as stent retrievers. Alternatively, the access systems described herein may also be useful for access to other parts of the body outside the vasculature. Similarly, where the working device is described as being an expandable cerebral treatment device, stent retriever or self-expanding stent other interventional devices can be delivered using the delivery systems described herein.

Where the distal access catheter is described herein as an aspiration catheter it should not be limited to only aspiration. Similarly, where the catheter is described herein as a way to deliver a stent retriever or a stent it should not be limited as such. It should also be appreciated that the systems described herein can be used to perform procedures that incorporate a combination of treatments. For example, the catheter can be used for the delivery of a stent delivery system, optionally in the presence of aspiration through the catheter. As another example, a user may start out performing a first interventional procedure using the systems described herein, such as aspiration thrombectomy, and switch to another interventional procedure, such as delivery of a stent retriever or stent.

As used herein, “embolus” or “embolus material” or “embolic material” or “embolic region” refers to material within a zone of an occlusion site that is more dense or a relatively hard consistency that is preferably placed in contact with a distal end of an aspiration catheter to successfully perform aspiration embolectomy. The embolus may be a thrombus (a clot of blood) or other material that formed at a first blood vessel location (e.g., a coronary vessel), breaks loose, and travels through the circulation to a second blood vessel location. As used herein, “in situ thrombus” or “thrombus material” or “thrombotic material” or “thrombotic region” or “in situ clot material” or “clot material” refers to material within a zone of an occlusion site that accumulates in situ at the site of the embolus and is often less dense or relatively soft and fluid-like. As used herein, “organized thrombus” refers to in situ thrombus material or clot material that accumulates at the site of embolus and is more dense and less fluid-like than the in situ clot material.

As used herein, “an occlusion” or “an occlusion site” or “occlusive material” refers to the blockage that occurred as a result of an atherosclerotic lesion or embolus lodging within a vessel and disrupting blood flow through the vessel or a stenosis within a vessel or sinus. The occlusion or occlusive material can include both thrombus and embolus as well as another non-thrombotic narrowing of the vessel.

As used herein, “an aneurysm” refers to the ballooning out of a weakened section of vessel wall. A “cerebral aneurysm” or “intracranial aneurysm” refers to an aneurysm in a vessel of the brain.

While some implementations are described herein with specific regard to accessing a neurovascular anatomy for application of aspiration, the systems and methods described herein should not be limited to this and may also be applicable to other uses such as the delivery of a stent deployment system. For example, the catheter systems described herein may be used to deliver working devices to the carotid artery or intracranial artery. Where the phrase “distal access catheter” or “aspiration catheter” is used herein that the catheter can be used for aspiration, the delivery of fluids to a treatment site or as a support catheter, or distal access providing a conduit that facilitates and guides the delivery or exchange of other devices such as a guidewire or interventional devices such as stents or stent retrievers.

The devices and systems described herein are related to and can be used in combination and in the alternative with the devices and systems described in U.S. Pat. No. 10,327,790, filed Aug. 3, 2012; U.S. Pat. No. 9,561,345, filed Dec. 19, 2014; U.S. Pat. No. 9,820,761, filed Feb. 4, 2016; U.S. Pat. No. 11,020,133, filed on Jan. 9, 2018; U.S. Pat. No. 10,799,663, filed on Jan. 19, 2018; U.S. Publication No. 2019/0351182, filed May 16, 2019; U.S. Application Serial No. 16/684,324, filed Nov. 14, 2019; and U.S. Publication No. 2020/0289136, filed Jun. 2, 2020. The disclosures of each of these publications and applications are incorporated by reference herein in their entireties.

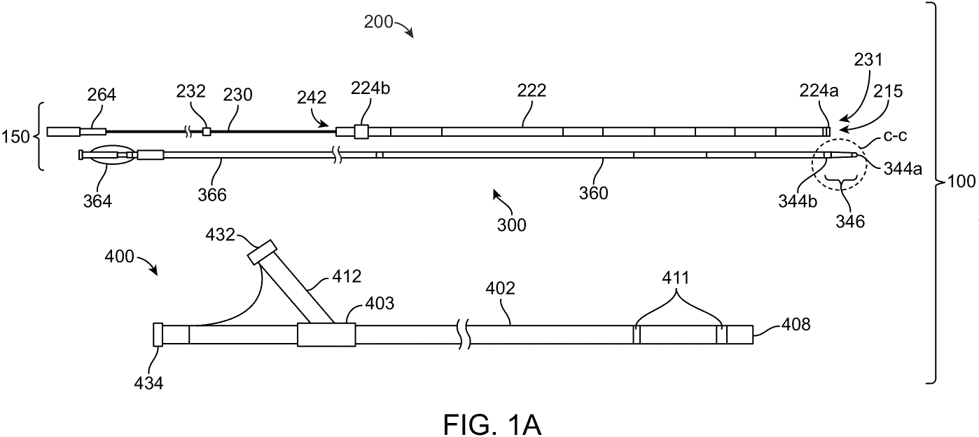

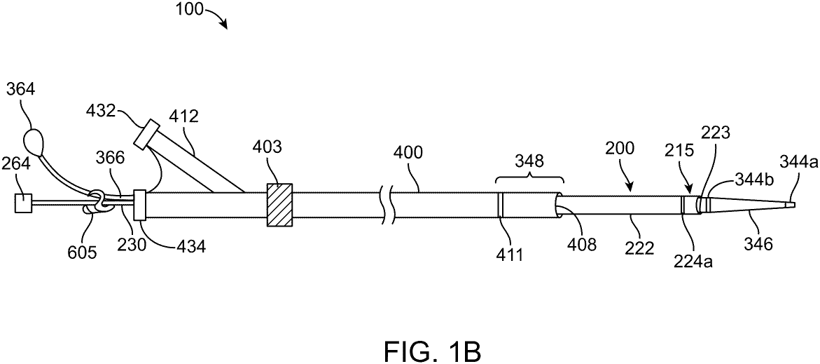

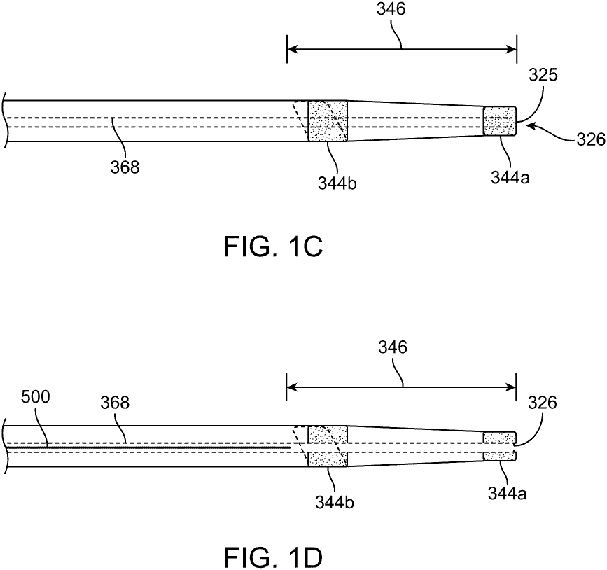

FIGS. 1A-1B illustrate an implementation of a distal access system 100 including devices for accessing and treating a cerebral occlusion such as by deploying a stent. FIG. 1A is an exploded view of an implementation of a catheter system and FIG. 1B is an assembled view of the catheter system of FIG. 1A. FIG. 1C is a detailed view of the catheter advancement element 300 of FIG. 1A taken along circle C-C. FIG. 1D is a detailed view of a catheter advancement element having a parked guidewire 500 in the lumen 368 so that a distal end of the guidewire 500 is positioned proximal to the distal opening 326 of the lumen 368. The distal access system 100 is capable of providing quick and simple access to distal target anatomy, particularly the tortuous anatomy of the cerebral vasculature. The system 100 can be a single operator system such that each of the components and systems can be delivered and used together by one operator through a single point of manipulation requiring minimal hand movements. As will be described in more detail below, all wire and catheter manipulations can occur at or in close proximity to a single rotating hemostatic valve (RHV) or more than a single RHV co-located in the same device.

The system 100 can include one or more catheter systems 150, each having a catheter 200 and a catheter advancement element 300. The catheter system 150 is configured to be advanced through an access guide sheath 400. The catheter 200 is configured to be received through the guide sheath 400 and is designed to have exceptional deliverability. The catheter 200 can, but need not, be a spined, distal access catheter co-axial with a lumen of the guide sheath 400 thereby providing a step-up in inner diameter within the conduit. The catheter need not include the proximal control element and instead can be a non-spined, conventional catheter having a uniform diameter. The catheter 200 can be a full length catheter. The catheter 200 can be delivered using a catheter advancement element 300 inserted through a lumen 223 of the catheter 200. The flexibility and deliverability of the distal access catheter 200 allow the catheter 200 to take the shape of the tortuous anatomy and avoids exerting straightening forces creating new anatomy. The distal access catheter 200 is capable of this even in the presence of the catheter advancement element 300 extending through its lumen. Thus, the flexibility and deliverability of the catheter advancement element 300 is on par or better than the flexibility and deliverability of the distal luminal portion 222 of the distal access catheter 200 in that both are configured to reach the middle cerebral artery (MCA) circulation without straightening out the curves of the anatomy along the way.

The system 100 can be a distal access system that can create a variable length from point of entry at the percutaneous arteriotomy (e.g. the femoral artery or other point of entry) to the target control point of the distal catheter. Conventional distal access systems for neurointervention typically include a long guide sheath or guide catheter placed through a shorter “introducer” sheath (e.g. 11-30 cm in length) at the groin. The long guide sheath is typically positioned in the ICA to support neurovascular interventions including stroke embolectomy (sometimes referred to as “thrombectomy”). For added support, these can be advanced up to the bony terminal petrous and rarely into the cavernous or clinoid or supraclinoid terminal ICA when possible. In some implementations, the guide sheath can be positioned lower within the carotid artery depending on whether the carotid is occlusive and being treated. To reach targets in the M1 or M2 distribution with devices for mechanical thrombectomy, such as devices for manual aspiration thrombectomy (MAT), stent retriever (SR), aspiration first pass technique (ADAPT) and “Solumbra” (Aspiration + SR), an additional catheter may be inserted through the long guide catheter. These catheters are typically large-bore aspiration catheters that can be, for example 130 cm in length or longer. As will be described in more detail below, the distal access systems 100 described herein can be shorter, for example, only 115 cm in length when taken as a system as measured from the access point, typically the common femoral artery. Additionally, the single operator can use the systems described herein by inserting them through a single rotating hemostatic valve (RHV) 434 on the guide sheath 400 or more than one RHV co-located in the same device such as a dual-headed RHV. Thus, what was once a two-person procedure can be a one-person procedure.

Still with respect to FIGS. 1A-1B, the distal access system 100 can include an access guide sheath 400 having a body 402 through which a working lumen extends from a proximal hemostasis valve 434 coupled to a proximal end region 403 of the body 402 to a distal opening 408 of a distal end region. The working lumen is configured to receive the catheter 200 therethrough such that a distal end of the catheter 200 can extend beyond a distal end of the sheath 400 through the distal opening 408. The guide sheath 400 can be used to deliver the catheters described herein as well as any of a variety of working devices known in the art. For example, the working devices can be configured to provide thrombotic treatments and can include large-bore catheters for aspiration embolectomy (sometimes referred to as thrombectomy), advanced catheters, wires, balloons, stents, stent delivery systems, retrievable structures such as coil-tipped retrievable stents “stent retriever”.

The sheath body 402 can extend from a proximal furcation or rotating hemostatic valve (RHV) 434 at a proximal end region 403 to a distal end opening 408 of the body 402. The proximal RHV 434 may include one or more lumens molded into a connector body to connect to the working lumen of the body 402 of the guide sheath 400. The working lumen can receive the catheter 200 and/or any of a variety of working devices for delivery to a target anatomy. The RHV 434 can be constructed of thick-walled polymer tubing or reinforced polymer tubing. The RHV 434 allows for the introduction of devices through the guide sheath 400 into the vasculature, while preventing or minimizing blood loss and preventing air introduction into the guide sheath 400. The RHV 434 can be integral to the guide sheath 400 or the guide sheath 400 can terminate on a proximal end in a female Luer adaptor to which a separate hemostasis valve component, such as a passive seal valve, a Tuohy-Borst valve or RHV may be attached. The RHV 434 can have an adjustable opening that is open large enough to allow removal of devices that have adherent clot on the distal end opening 408 without causing the clot to dislodge at the RHV 434 during removal. Alternately, the RHV 434 can be removable such as when a device is being removed from the sheath 400 to prevent clot dislodgement at the RHV 434. The RHV 434 can be a dual RHV or a multi-head RHV.

The RHV 434 can form a Y-connector on the proximal end region 403 of the sheath 400 such that the first port of the RHV 434 can be used for insertion of a working catheter into the working lumen of the sheath 400 and a second port into arm 412 can be used for another purpose. For example, a syringe or other device can be connected at arm 412 via a connector 432 to deliver a forward drip, a flush line for contrast agent or saline injections through the body 402 with or without a catheter toward the distal end opening 408 and into the target anatomy. Arm 412 can also connect to a vacuum source. The vacuum source can be an active source of aspiration such as an aspiration pump, a regular or locking syringe, a hand-held aspirator, hospital suction, or the like, configured to draw suction through the working lumen. In an embodiment, the vacuum source is a locking syringe (for example a VacLok Syringe) attached to a flow controller. The user can pull the plunger on the syringe back into a locked position while the connection to the flow line is closed prior to an embolectomy step of the procedure. During the procedure when the distal-most end 215 of the catheter 200 is near or at the proximal face of the occlusion 115 and the catheter advancement element 300 is removed from the lumen of the catheter 200, the user may open the connection to the aspiration syringe. This allows for a maximum communication of aspiration force being applied through the working lumen of the sheath 400 and any catheter extending through the sheath 400 that in turn is in communication with the vessel at its distal end. A single user at the single, shared source can apply the aspiration in a rapid fashion. In another implementation, the arm 412 can be connected to a vacuum source that is a pump configured to apply a constant or variable aspiration pressure through the working lumen of the guide sheath 400. The single, shared source of aspiration is sufficient to draw aspiration through the entire system 100, even when multiple aspiration catheters 200 are nested within one another through the working lumen of the guide sheath 400. The arm 412 can also allow the guide sheath 400 to be flushed with saline or radiopaque contrast agent during a procedure. The working lumen can extend from the distal end opening 408 to a working proximal port of the proximal end region 403 of the sheath body 402.

Contrast agent can be injected through the guide sheath 400 into the vessel to visualize the occlusion site by angiogram. For example, the guide sheath 400 can be positioned so that at least a portion is positioned within the carotid artery. The contrast agent may be injected through the sheath 400 once positioned in this location. Contrast agent can also be injected through one or more catheters inserted through the guide sheath 400. A baseline angiogram can be obtained, for example in the anterior/posterior (AP) and/or lateral views, prior to device insertion to assess occlusion location by injection of contrast media through the sheath 400 with fluoroscopic visualization. Fluoroscopic visualization may continue as the catheter system is advanced and subsequent angiograms can be captured periodically to assess reperfusion. The baseline angiogram image can be superimposed, such as with digital subtraction angiography, so that the vasculature and/or occlusion site are visible while the catheter system is advanced.

Once the catheter system 150 is advanced into position (the positioning will be described in more detail below), the catheter advancement element 300 can be withdrawn and removed from the system. In some implementations, the catheter 200 can be used as a support catheter to deliver a stent to the occlusion site (e.g., within the carotid or a cerebral artery) as will be described elsewhere herein.

In an implementation, the guide sheath 400 includes one or more radiopaque markers 411. The radiopaque markers 411 can be disposed near the distal end opening 408. For example, a pair of radiopaque bands may be provided. The radiopaque markers 411 or markers of any of the system components can be swaged, painted, embedded, or otherwise disposed in or on the body. In some implementations, the radiopaque markers include a barium polymer, tungsten polymer blend, tungsten-filled or platinum-filled marker that maintains flexibility of the devices and improves transition along the length of the component and its resistance to kinking. In some implementations, the radiopaque markers are a tungsten-loaded PEBAX or polyurethane that is heat welded to the component.

The guide sheath markers 411 are shown in the figures as rings around a circumference of one or more regions of the body 402. However, the markers 411 can have other shapes or create a variety of patterns that provide orientation to an operator regarding the position of the distal opening 408 within the vessel. Accordingly, an operator may visualize a location of the distal opening 408 under fluoroscopy to confirm that the distal opening 408 is directed toward a target anatomy where a catheter 200 is to be delivered. For example, radiopaque marker(s) 411 allow an operator to rotate the body 402 of the guide sheath 400 at an anatomical access point, e.g., a groin of a patient, such that the distal opening provides access to an ICA by subsequent working device(s), e.g., catheters and wires advanced to the ICA. In some implementations, the radiopaque marker(s) 411 include platinum, gold, tantalum, tungsten or any other substance visible under an x-ray fluoroscope. Any of the various components of the systems described herein can incorporate radiopaque markers.

Still with respect to FIGS. 1A-1B, the catheter 200 can include a relatively flexible, distal luminal portion 222 coupled to a stiffer, kink-resistant proximal extension or proximal control element 230. The term “control element” as used herein can refer to a proximal region configured for a user to cause pushing movement in a distal direction as well as pulling movement in a proximal direction. The control elements described herein may also be referred to as spines, tethers, push wires, push tubes, or other elements having any of a variety of configurations. The proximal control element 230 can be a hollow or tubular element. The proximal control element 230 can also be solid and have no inner lumen, such as a solid rod, ribbon or other solid wire type element. Generally, the proximal control elements described herein are configured to move its respective component (to which it may be attached or integral) in a bidirectional manner through a lumen.

A single, inner lumen 223 extends through the luminal portion 222 between a proximal end and a distal end of the luminal portion 222 (the lumen 223 is visible in FIG. 1B). In some implementations, a proximal opening 242 into the lumen 223 can be located near where the proximal control element 230 coupled with the distal luminal portion 222. In other implementations, the proximal opening 242 into the lumen 223 is at a proximal end region of the catheter 200. A distal opening 231 from the lumen 223 can be located near or at the distal-most end 215 of the luminal portion 222. The inner lumen 223 of the catheter 200 can have a first inner diameter and the working lumen of the guide sheath 400 can have a second, larger inner diameter. Upon insertion of the catheter 200 through the working lumen of the sheath 400, the lumen 223 of the catheter 200 can be configured to be fluidly connected and contiguous with the working lumen of the sheath 400 such that fluid flow into and/or out of the system 100 is possible, such as by applying suction from a vacuum source coupled to the system 100 at a proximal end. The combination of sheath 400 and catheter 200 can be continuously in communication with the bloodstream during aspiration at the proximal end with advancement and withdrawal of catheter 200.

The distal luminal portion 222 of the catheter 200 can have one or more radiopaque markings 224. A first radiopaque marker 224a can be located near the distal-most end 215 to aid in navigation and proper positioning of the distal-most end 215 under fluoroscopy. Additionally, a proximal region of the catheter 200 may have one or more proximal radiopaque markers 224b so that the overlap region 348 can be visualized as the relationship between a radiopaque marker 411 on the guide sheath 400 and the radiopaque marker 224b on the catheter 200. The proximal region of the catheter 200 may also have one or more radiopaque markings providing visualization, for example, near the proximal opening 242 into the single lumen 223 of the catheter 200 as will be described in more detail below. In an implementation, the two radiopaque markers (marker 224a near the distal-most end 215 and a more proximal marker 224b) are distinct to minimize confusion of the fluoroscopic image, for example the catheter proximal marker 224b may be a single band and the marker 411 on the guide sheath 400 may be a double band and any markers on a working device delivered through the distal access system can have another type of band or mark. The radiopaque markers 224 of the distal luminal portion 222, particularly those near the distal end region navigating extremely tortuous anatomy, can be relatively flexible such that they do not affect the overall flexibility of the distal luminal portion 222 near the distal end region. The radiopaque markers 224 can be tungsten-loaded or platinum-loaded markers that are relatively flexible compared to other types of radiopaque markers used in devices where flexibility is not paramount. In some implementations, the radiopaque marker can be a band of tungsten-loaded PEBAX having a durometer of Shore 35D.

The proximal control element 230 can include one or more markers 232 to indicate the overlap between the distal luminal portion 222 of the catheter 200 and the sheath body 402 as well as the overlap between the distal luminal portion 222 of the catheter 200 and other interventional devices that may extend through the distal luminal portion 222. At least a first mark can be an RHV proximity marker positioned so that when the mark is aligned with the sheath proximal hemostasis valve 434 during insertion of the catheter 200 through the guide sheath 400, the catheter 200 is positioned at the distal-most position with the minimal overlap length needed to create the seal between the catheter 200 and the working lumen. At least a second marker 232 can be a Fluoro-saver marker that can be positioned on the control element 230 and located a distance away from the distal-most end 215 of the distal luminal portion 222. In some implementations, a marker 232 can be positioned about 100 cm away from the distal-most end 215 of the distal luminal portion 222. The markers 232 can be positioned on the catheter so that one or more markers are visible to an operator outside the patient (and outside the guide sheath 400) during use. One or more markers can also be visible to an operator inside the patient (and inside the guide sheath 400 or beyond a distal end of the guide sheath 400) during use such that they are visualized under fluoroscopy.

The distal access catheter 200 can be used to deliver endovascular scaffolding devices to the intracranial anatomy or may be used as an aspiration catheter. It is desirable to deliver a catheter with as large a bore as clinically possible to achieve an optimal result for aspiration thrombectomy procedures, for example, a distal access catheter having an inner diameter of at least about 0.070” or 0.088” or greater. For delivery of endovascular devices, the delivery catheter can have a large-enough bore to deliver the desired devices. The ability to deliver larger-bore access catheters allows for improved endovascular devices, which will be described more fully below. In this latter case, there may not be a need to deliver “as large as clinically possible” as in aspiration thrombectomy procedures. For delivery of endovascular devices, the distal access catheter 200 may be as small as 0.054” inner diameter or even smaller depending on the desired size of the endovascular device being delivered. These smaller distal access catheters 200 can be associated with a corresponding sized catheter advancement element 300.

Although the catheter advancement element 300 is described herein in reference to catheter 200 it can be used to advance other catheters and it is not intended to be limiting to its use. For example, the catheter advancement element 300 can be used to deliver a 5MAX Reperfusion Catheter (Penumbra, Inc. Alameda, CA), REACT aspiration catheter (Medtronic), or Sophia Plus aspiration catheter (Terumo) for clot removal in patients with acute ischemic stroke or other reperfusion catheters known in the art. In an embodiment, the catheter advancement element 300 is sized-matched to a large bore catheter 200 that can be about 0.088” inner diameter.

Still with respect to FIGS. 1A-1B and also FIG. 1C, the catheter advancement element 300 can include a non-expandable, flexible elongate body 360 and a proximal portion 366 extending proximally from the elongate body 360. The catheter advancement element 300 and the catheter 200 described herein may be configured for rapid exchange or over-the-wire methods. For example, the flexible elongate body 360 can be a tubular portion extending the entire length of the catheter advancement element 300 and can have a proximal opening from the lumen 368 of the flexible elongate body 360 that is configured to extend outside the patient’s body during use. Alternatively, the tubular portion can have a proximal opening positioned such that the proximal opening remains inside the patient’s body during use. The proximal portion 366 can be a proximal element coupled to a distal tubular portion 360 and extending proximally therefrom. A proximal opening from the tubular portion 360 can be positioned near where the proximal element 366 couples to the tubular portion 360. Alternatively, the proximal portion 366 can be a proximal extension of the tubular portion 360 having a length that extends to a proximal opening near a proximal terminus of the catheter advancement element 300 (i.e. outside a patient’s body). A luer 364 can be coupled to the proximal portion 366 at the proximal end region so that tools such as a guidewire can be advanced through the lumen 368 of the catheter advancement element 300. A syringe or other component can be coupled to the luer 364 in order to draw a vacuum and/or inject fluids through the lumen 368. The syringe coupled to the luer 364 can also be used to close off the lumen of the catheter advancement element 300 to maximize the piston effect described elsewhere herein.

The configuration of the proximal portion 366 can vary. In some implementations, the proximal portion 366 is simply a proximal extension of the flexible elongate body 360 that does not change significantly in structure but changes significantly in flexibility. For example, the proximal portion 366 transitions from the very flexible distal regions of the catheter advancement element 300 towards less flexible proximal regions of the catheter advancement element 300. The proximal portion 366 provides a relatively stiff proximal end suitable for manipulating and torqueing the more distal regions of the catheter advancement element 300. In other implementations, the proximal portion 366 is a metal reinforced segment. The metal reinforced segment can be positioned a distance away from the distal end of the elongate body. For example, the metal reinforced segment can terminate or be about 50 cm from the distal end. The metal reinforced segment can have an inner diameter of about 0.021” and an outer diameter of about 0.027”. The metal reinforced segment can be a spine. The metal reinforced segment can be a hypotube. In other implementations, the proximal portion 366 is a hypotube coupled to the elongate body 360. The hypotube may be exposed or may be coated by a polymer. In still further implementations, the proximal portion 366 may be a tubular polymer portion reinforced by a coiled ribbon or braid. The proximal portion 366 can have the same outer diameter as the flexible elongate body or can have a smaller outer diameter as the flexible elongate body.

The proximal portion 366 need not include a lumen. For example, the proximal portion 366 can be a solid rod, ribbon, or wire have no lumen extending through it that couples to the tubular elongate body 360. Where the proximal portion 366 is described herein as having a lumen, it should be appreciated that the proximal portion 366 can also be solid and have no lumen. The proximal portion 366 is generally less flexible than the elongate body 360 and can transition to be even more stiff towards the proximal-most end of the proximal portion 366. Thus, the catheter advancement element 300 can have an extremely soft and flexible distal end region 346 that transitions proximally to a stiff proximal portion 366 well suited for pushing the distal elongate body 360. The distal elongate body 360 of the catheter advancement element 300 is substantially non-reinforced, fully polymeric body. In other words, the distal elongate body 360 can include no higher durometer element (i.e., metal coil or braid, stiff polymer, etc.) such that the stiff proximal portion 366 can be used for pushing the distal elongate body 360, but may not necessarily be useful in torqueing the distal elongate body 360. In other implementations, the catheter advancement element 300 can be reinforced distally such that the proximal portion 366 can be used for pushing and also torqueing the distal elongate body 360. In some implementations, the catheter advancement element 300 can be inserted and advanced through the catheter by pushing on the proximal portion 366, but the advancement of the catheter system as a whole is achieved primarily by pushing on the combination of the catheter 200 and the catheter advancement element 300.

The elongate body 360 can be received within and extended through the internal lumen 223 of the distal luminal portion 222 of the catheter 200 (see FIG. 1B). The elongate body 360 or tubular portion can have an outer diameter. The outer diameter of the tubular portion can have at least one snug point. The at least one snug point provides a close fit between the elongate body 360 and the distal luminal portion 222 that minimizes a distal lip or edge at the distal end of the catheter 200, but that still allows for movement relative to one another so as to allow a user to achieve a desired extension or withdrawal of the catheter advancement element 300 relative to the catheter 200 or the catheter 200 relative to the catheter advancement element 300. The snug point allows for movement between the catheters upon application of a relatively small load so as to avoid any negative impact on usability within a patient. A difference between the inner diameter of the catheter 200 and the outer diameter of the tubular portion at the snug point can be no more than about 0.015″ (0.381 mm), or can be no more than about 0.010″ (0.254 mm), for example, from about 0.003″ (0.0762 mm) up to about 0.012″ (0.3048 mm), preferably about 0.005″ (0.127 mm) to about 0.010″ (0.254 mm), and more preferably about 0.007″ (0.1778 mm) to about 0.009″ (0.2286 mm).

As will be described in more detail below, the catheter advancement element 300 can also include a distal end region 346 located distal to the at least one snug point of the tubular portion. The distal end region 346 can have a length and taper along at least a portion of the length. The distal end region 346 of the catheter advancement element 300 can be extended beyond the distal end of the catheter 200 as shown in FIG. 1B. The proximal portion 366 of the catheter advancement element 300 or proximal extension is coupled to a proximal end region of the elongate body 360 and extends proximally therefrom. The proximal portion 366 can be less flexible than the elongate body 360 and configured for bi-directional movement of the elongate body 360 of the catheter advancement element 300 within the luminal portion 222 of the catheter 200, as well as for movement of the catheter system 100 as a whole. The elongate body 360 can be inserted in a coaxial fashion through the internal lumen 223 of the luminal portion 222. The outer diameter of at least a region of the elongate body 360 can be sized to substantially fill at least a portion of the internal lumen 223 of the luminal portion 222.

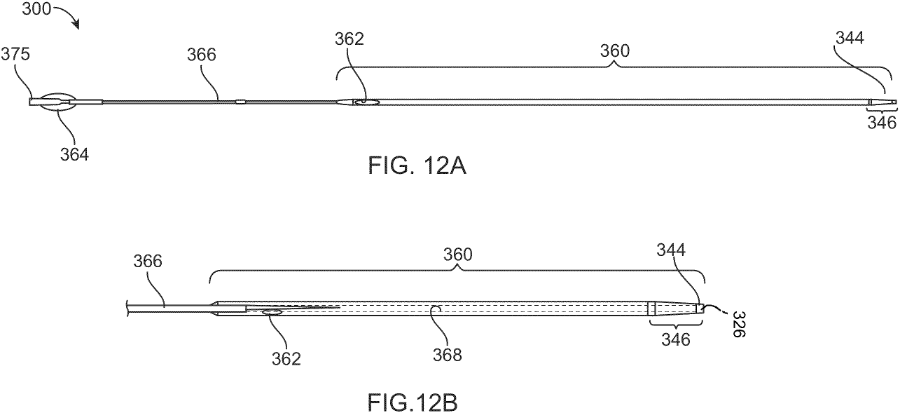

The overall length of the catheter advancement element 300 (e.g. between the proximal end through to the distal-most tip) can vary, but generally is long enough to extend through the support catheter 200 plus at least a distance beyond the distal end of the support catheter 200 while at least a length of the proximal portion 366 remains outside the proximal end of the guide sheath 400 and outside the body of the patient. In some implementations, the overall length of the catheter advancement element 300 is about 145 to about 150 cm and has a working length of about 140 cm to about 145 cm from a proximal tab 364 or hub 375 (shown in FIG. 12A) to the distal-most end 325. The elongate body 360 can have a length that is at least as long as the luminal portion 222 of the catheter 200 although the elongate body 360 can be shorter than the luminal portion 222 so long as at least a minimum length remains inside the luminal portion 222 when a distal portion of the elongate body 360 is extended distal to the distal end of the luminal portion 222 to form a snug point or snug region with the catheter. In some implementations, this minimum length of the elongate body 360 that remains inside the luminal portion 222 when the distal end region 346 is positioned at its optimal advancement configuration is at least about 5 cm, at least about 6 cm, at least about 7 cm, at least about 8 cm, at least about 9 cm, at least about 10 cm, at least about 11 cm, or at least about 12 cm up to about 50 cm. In some implementations, the shaft length of the distal luminal portion 222 can be about 35 cm up to about 75 cm and shorter than a working length of the guide sheath and the insert length of the elongate body 360 can be at least about 45 cm, 46 cm, 47 cm, 48 cm, 48.5 cm, 49 cm, 49.5 cm up to about 85 cm.

The length of the elongate body 360 can allow for the distal end of the elongate body 360 to reach cerebrovascular targets or occlusions within, for example, segments of the internal carotid artery including the cervical (C1), petrous (C2), lacerum (C3), cavernous (C4), clinoid (C5), ophthalmic (C6), and communicating (C7) segments of the internal carotid artery (ICA) as well as branches off these segments including the M1 or M2 segments of the middle cerebral artery (MCA), anterior cerebral artery (ACA), anterior temporal branch (ATB), and/or posterior cerebral artery (PCA). The distal end region of the elongate body 360 can reach these distal target locations while the proximal end region of the elongate body 360 remains proximal to or below the level of severe turns along the path of insertion. For example, the entry location of the catheter system can be in the femoral artery and the target occlusion location can be distal to the right common carotid artery, such as within the M1 segment of the middle cerebral artery on the right side. The proximal end region of the elongate body 360 where it transitions to the proximal portion 366 can remain within a vessel that is proximal to severely tortuous anatomy such as the carotid siphon, the right common carotid artery, the brachiocephalic trunk, the take-off into the brachiocephalic artery from the aortic arch, the aortic arch as it transitions from the descending aorta. This avoids inserting the stiffer proximal portion 366, or the material transition between the stiffer proximal portion 366 and the elongate body 360, from taking the turn of the aortic arch or the turn of the brachiocephalic take-off from the aortic arch, which both can be very severe. The lengths described herein for the distal luminal portion 222 also can apply to the elongate body 360 of the catheter advancement element.

The proximal portion 366 can have a length that varies as well. In some implementations, the proximal portion 366 is about 90 cm up to about 95 cm. The distal portion extending distal to the distal end of the luminal portion 222 can include distal end region 346 that protrudes a length beyond the distal end of the luminal portion 222 during use of the catheter advancement element 300. The distal end region 346 of the elongate body 360 that is configured to protrude distally from the distal end of the luminal portion 222 during advancement of the catheter 200 through the tortuous anatomy of the cerebral vessels, as will be described in more detail below. The proximal portion 366 coupled to and extending proximally from the elongate body 360 can align generally side-by-side with the proximal control element 230 of the catheter 200. The arrangement between the elongate body 360 and the luminal portion 222 can be maintained during advancement of the catheter 200 through the tortuous anatomy to reach the target location for treatment in the distal vessels and aids in preventing the distal end of the catheter 200 from catching on tortuous branching vessels, as will be described in more detail below.

In some implementations, the elongate body 360 can have a region of relatively uniform outer diameter extending along at least a portion of its length and the distal end region 346 tapers down from the uniform outer diameter. The outer diameter of the elongate body 360 also can taper or step down in outer diameter proximally, for example near where the elongate body 360 couples or transitions to the proximal portion 366. The outer diameter of the elongate body 360 need not change in outer diameter near where the elongate body 360 couples to the proximal portion 366. In some implementations, the region of relatively uniform outer diameter can extend along a majority of the working length of the catheter advancement element 300 including the proximal portion 366. This first region of uniform outer diameter can transition to a second region of uniform outer diameter located distal to the first region. The transition can incorporate a smooth taper or step change in outer diameter between the two regions. The second region of uniform outer diameter having the larger size and located distal to the first region can be useful in filling a lumen of a larger bore catheter without the entire working length of the elongate body needing to have this larger size. In this embodiment, the elongate body 360 can have a distal taper changing in diameter from the second uniform diameter region towards the distal opening and a proximal taper changing in diameter from the second uniform diameter region towards the first region of uniform outer diameter.

Depending upon the inner diameter of the catheter 200, the difference between the inner diameter of catheter 200 and the outer diameter of the elongate body 360 along at least a portion of its length, such as at least 10 cm of its length, preferably at least 15 cm of its length can be no more than about 0.015″ (0.381 mm), such as within a range of about 0.003″ - 0.015″ (0.0762 mm - 0.381 mm) or between 0.006″ - 0.010″ (0.1524 mm - 0.254 mm). Thus, the clearance between the catheter 200 and the elongate body 360 can result in a space on opposite sides that is no more than about 0.008″ (0.2032 mm), or can be no more than about 0.005″ (0.127 mm), for example, from about 0.001″ up to about 0.006″ (0.0254 mm - 0.1524 mm), preferably about 0.002″ to about 0.005″ (0.0508 mm - 0.127 mm), and more preferably about 0.003″ to about 0.005″ (0.0762 mm - 0.0508 mm).

The catheter advancement element 300 has a large outer diameter and a relatively small inner diameter, particularly when a guidewire extends into or through the lumen of the catheter advancement element 300. The lumen of the catheter advancement element 300 substantially filled by the guidewire and/or liquid creates a closed system with the catheter 200. The catheter advancement element 300 substantially fills or is substantially occlusive to the catheter 200 creating a piston arrangement within the catheter lumen. Withdrawing the occlusive catheter advancement element 300 through the catheter lumen creates an internal vacuum like a plunger in a syringe barrel. The internal vacuum created within the distal end region of the catheter 200 can draw embolic material towards and/or through the distal end 215 of the catheter 200 positioned at or near the face of the embolus 115. Further, the catheter system as it is advanced through the tortuous neuroanatomy can store energy or forces, for example, in the compression of the catheter 200 before the catheter advancement element 300 is withdrawn. The extreme tortuosity of the intracerebral vasculature, particularly around the bony structures of the skull can require more severe force to traverse in combination with the dramatic transition in the size between vessels to reach the occlusion site, such as the large aorta and 1-3 mm sized target vessel, can cause stored forces or energy in a catheter. Withdrawal of the catheter advancement element 300 can release this stored energy causing distally-directed movement of the distal catheter portion 222. A user may exploit the distally-directed movement of the distal catheter portion 222 towards the embolus 115 to atraumatically nest, seat, and/or embed the distal end 215 of the catheter 200 with the proximal face of the embolus 115 for positioning of the catheter 200 relative to the embolus. The elongate body 360 can have an overall shape profile from proximal end to distal end that transitions from a first outer diameter having a first length to a tapering outer diameter having a second length. The first length of this first outer diameter region (i.e. the snug-fitting region between the distal luminal portion 222 and the elongate body 360) can be at least about 5 cm, or 10 cm, up to about 50 cm. In other implementations, the snug-fitting region can extend from the proximal tab or luer 364 substantially to the tapered distal end region 346 which depending on the length of the catheter advancement element 300, can be up to about 170 cm.

The length of the tapering outer diameter of the distal end region 346 can be about 0.5 cm to about 5 cm, about 1 cm to about 4 cm, or about 1.5 cm to about 3 cm. The distal end region 346 of the elongate body 360 can also be shaped with or without a taper. When the catheter advancement element 300 is inserted through the catheter 200, this distal end region 346 is configured to extend beyond and protrude out through the distal-most end 215 of the luminal portion 222 whereas the more proximal region of the body 360 (i.e. the first length described above) remains within the luminal portion 222.

As mentioned, the distal-most end 215 of the luminal portion 222 can be blunt and have no change in the dimension of the outer diameter whereas the distal end region 346 can be tapered providing an overall elongated tapered geometry of the catheter system. The outer diameter of the elongate body 360 also approaches the inner diameter of the luminal portion 222 such that the step-up from the elongate body 360 to the outer diameter of the luminal portion 222 is minimized. Minimizing this step-up prevents issues with the lip formed by the distal end of the luminal portion 222 catching on the tortuous neurovasculature, such as around the carotid siphon near the ophthalmic artery branch, when the distal end region 346 in combination with the distal end region of the catheter 200 bends and curves along within the vascular anatomy. In some implementations, the inner diameter of the luminal portion 222 can be at least about 0.052″ (1.321 mm), about 0.054″ (1.372 mm) and the maximum outer diameter of the elongate body 360 can be about 0.048″ (1.219 mm) such that the difference between them is about 0.006″ (0.1524 mm). In some implementations, the inner diameter of the luminal portion 222 can be about 0.070″ (1.778 mm) and the maximum outer diameter of the elongate body 360 can be about 0.062″ (1.575 mm) such that the difference between them is about 0.008″ (0.2032 mm). In some implementations, the inner diameter of the luminal portion 222 can be about 0.088″ (2.235 mm) and the maximum outer diameter of the elongate body 360 can be about 0.080″ (2.032 mm) such that the difference between them is about 0.008″ (0.2032 mm). In some implementations, the inner diameter of the luminal portion 222 can be about 0.072″ (1.829 mm) and the maximum outer diameter of the elongate body 360 is about 0.070” (1.778 mm) such that the difference between them is only 2 thousandths of an inch (0.002″/ 0.0508 mm). In other implementations, the maximum outer diameter of the elongate body 360 is about 0.062″ (1.575 mm) such that the difference between them is about 0.010″ (0.254 mm). Despite the outer diameter of the elongate body 360 extending through the lumen of the luminal portion 222, the luminal portion 222 and the elongate body 360 extending through it in co-axial fashion are flexible enough to navigate the tortuous anatomy leading to the level of M1 or M2 arteries without kinking and without damaging the vessel. It is preferred to deliver a catheter that is as large in inner diameter as possible so that large aspiration forces can be delivered to the site of the occlusion in aspiration-only treatment without the risk of the catheter lumen getting clogged. The large inner diameter of the catheter is also helpful for the delivery of larger-sized stents. Further, the single point of access at the guide sheath RHV means the stent delivery system will always be long enough. In preferred embodiments, the catheter delivered to the treatment site has a lumen size that is at least about 0.088″.

The dimensions provided herein are approximate and each dimensions may have an engineering tolerance or a permissible limit of variation. Use of the term “about,” “approximately,” or “substantially” are intended to provide such permissible tolerance to the dimension being referred to. Where “about” or “approximately” or “substantially” is not used with a particular dimension herein that that dimension need not be exact.

The length of the tapered distal end region 346 can vary. In some implementations, the length of the distal end region 346 can be in a range of between about 0.50 cm to about 5.0 cm from the distal-most end of the elongate body 360 or between about 1.0 cm to about 4.0 cm, or about 1.5 cm to about 3 cm, or between 2.0 and about 2.5 cm. In some implementations, the length of the distal end region 346 varies depending on the inner diameter of the catheter 200 with which the catheter advancement element 300 is to be used. For example, the length of the distal end region 346 can be as shorter (e.g. 1.2 cm) for a catheter advancement element 300 sized to be used with a catheter 200 having an inner diameter of about 0.054″ (1.372 mm) and can be longer (e.g. 2.5 cm) for a catheter advancement element 300 sized to be used with a catheter 200 having an inner diameter of about 0.088″ (2.235 mm). The distal end region 346 can be a constant taper from the larger outer diameter of the elongate body 360 (e.g. the distal end of the marker 344b) down to a second smaller outer diameter at the distal-most terminus (e.g. the proximal end of the marker 344a) as shown in FIG. 1C. In some implementations, the constant taper of the distal end region 346 can be from about 0.048″ outer diameter down to about 0.031″ (0.787 mm) outer diameter over a length of about 1 cm. In some implementations, the constant taper of the distal end region 346 can be from 0.062″ (1.575 mm) outer diameter to about 0.031″ (0.787 mm) outer diameter over a length of about 2 cm. In still further implementations, the constant taper of the distal end region 346 can be from 0.080″ (2.032 mm) outer diameter to about 0.031″ (0.787 mm) outer diameter over a length of about 2.5 cm. The length of the constant taper of the distal end region 346 can vary, for example, between 0.8 cm to about 2.5 cm, or between 1 cm and 3 cm, or between 2.0 cm and 2.5 cm. The angle of the taper can vary depending on the outer diameter of the elongate body 360. For example, the angle of the taper can be between 0.9 to 1.6 degrees relative to horizontal. The angle of the taper can be between 2-3 degrees from a center line of the elongate body 360. The length of the taper of the distal end region 346 can be between about 5 mm to 20 mm or about 20 mm to about 50 mm.

The elongate body 360 of the catheter advancement element 300 can have a lumen 368 with an inner diameter that does not change over the length of the elongate body even in the presence of the tapering of the distal end region 346. Thus, the inner diameter of the lumen 368 extending through the tubular portion of the catheter advancement element 300 can remain uniform and the wall thickness of the distal end region 346 can decrease to provide the taper. The wall thickness can thin distally along the length of the taper. Thus, the material properties in combination with wall thickness, angle, length of the taper can all contribute to the overall maximum flexibility of the distal-most end of the distal end region 346. The catheter advancement element 300 undergoes a transition in flexibility from the distal-most end towards the snug point where it achieves an outer diameter that is no more than about 0.010″ (0.254 mm) different from the inner diameter of the catheter 200.

The inner diameter of the elongate body 360 can be constant along its length even where the single lumen passes through the tapering distal end region 346. Alternatively, the inner diameter of the elongate body 360 can have a first size through the tapering distal end region 346 and a second, larger size through the cylindrical section of the elongate body 360. The cylindrical section of the elongate body 360 can have a constant wall thickness or a wall thickness that varies to a change in inner diameter of the cylindrical section. As an example, the outer diameter of the cylindrical section of the elongate body 360 can be about 0.080″. The inner diameter of the elongate body 360 within the cylindrical section can be uniform along the length of the cylindrical section and can be about 0.019″. The wall thickness in this section, in turn, can be about 0.061″. As another example, the outer diameter of the cylindrical section of the elongate body 360 can again be between about 0.080″. The inner diameter of the elongate body 360 within the cylindrical section can be non-uniform along the length of the cylindrical section and can step-up from a first inner diameter of about 0.019″ to a larger second inner diameter of about 0.021″. The wall thickness, in turn, can be about 0.061” at the first inner diameter region and about 0.059″ at the second inner diameter region. The wall thickness of the cylindrical portion of the elongate body 360 can be between about 0.050″ to about 0.065″. The wall thickness of the tapered distal end region 346 near the location of the proximal marker band can be the same as the cylindrical portion (between about 0.050″ and about 0.065″) and become thinner towards the location of the distal marker band. As an example, the inner diameter at the distal opening from the single lumen can be about 0.020″ and the outer diameter at the distal opening (i.e. the outer diameter of the distal marker band) and be about 0.030″ resulting in a wall thickness of about 0.010″ compared to the wall thickness of the cylindrical portion that can be up to about 0.065″. Thus, the outer diameter of the distal tip 346 can taper as can the wall thickness. A wall thickness of the intermediate segment and an untapered portion of the tip segment can be about 0.050 inch to about 0.065 inch. The wall thickness of the intermediate segment and the untapered portion can be constant. The inner diameter of the intermediate segment and the tapered end region can be constant.

A tip segment of the flexible elongate body can have a tapered portion that tapers distally from a first outer diameter to a second outer diameter. The second outer diameter can be about ½ of the first outer diameter. The second outer diameter can be about 40% of the first outer diameter. The second outer diameter can be about 65% of the first outer diameter. The first outer diameter can be about 0.062″ up to about 0.080″. The second outer diameter can be about 0.031″. The second outer diameter can be about 50% of the first outer diameter, about 40% of the first outer diameter, or about 65% of the first outer diameter.

The length of the taper can also vary depending on the anatomy of the target region. The distal end region 346 can achieve its soft, atraumatic and flexible characteristic due to a material property other than due to a change in outer dimension to facilitate endovascular navigation to an occlusion in tortuous anatomy. Additionally or alternatively, the distal end region 346 of the elongate body 360 can have a transition in flexibility along its length. The most flexible region of the distal end region 346 can be its distal terminus. Moving along the length of the distal end region 346 from the distal terminus towards a region proximal to the distal terminus. For example, the distal end region 346 can be formed of a material having a Shore material hardness of no more than 35D or about 62A and transitions proximally to be less flexible near where it is formed of a material having a material hardness of no more than 55D and 72D up to the proximal portion 366, which can be a stainless steel hypotube, or a combination of a material property and tapered shape. The materials used to form the regions of the elongate body 360 can include PEBAX (such as PEBAX 25D, 35D, 55D, 69D, 72D) or a blend of PEBAX (such as a mix of 25D and 35D, 25D and 55D, 25D and 72D, 35D and 55D, 35D and 72D, 55D and 72D, where the blend ratios may range from 0.1% up to 50% for each PEBAX durometer), with a lubricious additive compound, such as Mobilize (Compounding Solutions, Lewiston, Maine). In some implementations, the material used to form a region of the elongate body 360 can be Tecothane 62A. Incorporation of a lubricious additive directly into the polymer elongate body means incorporation of a separate lubricious liner, such as a Teflon liner, is unnecessary. This allows for a more flexible element that can navigate the distal cerebral anatomy and is less likely to kink. Similar materials can be used for forming the distal luminal portion 222 of the catheter 200 providing similar advantages. The flexibility of the distal end region 346 can be achieved by a combination of flexible lubricious materials and tapered shapes. For example, the length of the distal end region 346 can be kept shorter than 2 cm - 3 cm, but maintain optimum deliverability due to a change in flexible material from distal-most end 325 towards a more proximal region a distance away from the distal-most end 325. In an implementation, the elongate body 360 is formed of PEBAX (polyether block amide) embedded silicone designed to maintain the highest degree of flexibility. The wall thickness of the distal end of the luminal portion 222 can also be made thin enough such that the lip formed by the distal end of the luminal portion 222 relative to the elongate body 360 is minimized.

The elongate body 360 has a benefit over a microcatheter in that it can have a relatively large outer diameter that is just 0.003″-0.010″ (0.0762 mm - 0.254 mm) smaller than the inner diameter of the distal luminal portion 222 of the catheter 200 and still maintaining a high degree of flexibility for navigating tortuous anatomy. When the gap between the two components is too tight (e.g. less than about 0.003″ (.0762 mm), the force needed to slide the catheter advancement element 300 relative to the catheter 200 can result in damage to one or both of the components and increases risk to the patient during the procedure. The gap results in too tight of a fit to provide optimum relative sliding. When the gap between the two components is too loose (e.g. greater than about 0.010″ / 0.254 mm), the distal end of the catheter 200 forms a lip that is prone to catch on carotid dissections or branching vessels during advancement through tortuous neurovasculature, such as around the carotid siphon where the ophthalmic artery branches off and the piston effect of withdrawal of the elongate body 360 can be decreased or lost.

The gap in ID/OD between the elongate body 360 and the distal luminal portion 222 can be in this size range (e.g. 0.003″ - 0.015″ (0.0762 mm - 0.381 mm) or between 0.006″ -0.010″ (0.152 mm -0.254 mm)) along a majority of their lengths. For example, the elongate body 360 can have a relatively uniform outer diameter that is between about 0.048″ (1.219 mm) to about 0.080″ (2.032 mm) from a proximal end region to a distal end region up to a point where the taper of the distal end region 346 begins. Similarly, the distal luminal portion 222 of the catheter 200 can have a relatively uniform inner diameter that is between about 0.054″ (1.372 mm) to about 0.088″ (2.235 mm) from a proximal end region to a distal end region. As such, the difference between their respective inner and outer diameters along a majority of their lengths can be within this gap size range of 0.003″ to 0.015″ (0.0762 mm - 0.381 mm). The distal end region 346 of the elongate body 360 that is tapered will have a larger gap size relative to the inner diameter of the distal luminal portion 222. During use, however, this tapered distal end region 346 is configured to extend distal to the distal end of the catheter 200 such that the region of the elongate body 360 having an outer diameter sized to match the inner diameter of the distal luminal portion 222 is positioned within the lumen of the catheter 200 such that it can minimize the lip at the distal end of the catheter 200.

The elongate body 360 can be formed of various materials that provide a suitable flexibility and lubricity. Example materials include high density polyethylene, 77A PEBAX, 33D PEBAX, 42D PEBAX, 46D PEBAX, 54D PEBAX, 69D PEBAX, 72D PEBAX, 90D PEBAX, and mixtures thereof or equivalent stiffness and lubricity material. In some implementations, the elongate body 360 is an unreinforced, non-torqueing catheter having a relatively large outer diameter designed to fill the lumen it is inserted through and a relatively small inner diameter to minimize any gaps at a distal-facing end of the device. In other implementations, at least a portion of the elongate body 360 can be reinforced to improve navigation and torqueing (e.g. braided reinforcement layer). The flexibility of the elongate body 360 can increase towards the distal end region 346 such that the distal region of the elongate body 360 is softer, more flexible, and articulates and bends more easily than a more proximal region. For example, a more proximal region of the elongate body can have a bending stiffness that is flexible enough to navigate tortuous anatomy such as the carotid siphon without kinking. If the elongate body 360 has a braid reinforcement layer along at least a portion of its length, the braid reinforcement layer can terminate a distance proximal to the distal end region 346. For example, the distance from the end of the braid to the distal-most end 325 can be about 10 cm to about 15 cm or from about 4 cm to about 10 cm or from about 4 cm up to about 15 cm.

In some implementations, the elongate body 360 can be generally tubular along at least a portion of its length such that it has a single lumen 368 extending parallel to a longitudinal axis of the catheter advancement element 300 (see FIGS. 1A-1D). In an implementation, the single lumen 368 of the elongate body 360 is sized to accommodate a guidewire, however use of the catheter advancement element 300 generally eliminates the need for a guidewire lead. Preferably, the assembled system includes no guidewire or a guidewire parked inside the lumen 368 retracted away from the distal opening. Guidewires are designed to be exceptionally flexible so that they deflect to navigate the severe turns of the anatomy. However, many workhorse guidewires have a stiffness along their longitudinal axis and/or are small enough in outer diameter that they find their own paths through an occlusion rather than slipping around the occlusion or get hung up on vessel wall dissections increasing the risk of perforations. In some cases, these guidewires can cause perforations and/or dissections of the vessel itself. Guidewires tend to get redirected into branches rather than remaining within the larger vessel. This makes them helpful for selecting a branch, but problematic for navigating tortuous anatomy and following the main flow of blood. Thus, even though the guidewire may have an outer diameter at its distal tip region that is small and very flexible at the distal tip, guidewires typically are incapable of atraumatically probing an occlusion or other structure such that the pose a risk of perforation with repeated advancement. Guidewires do not deflect upon encountering something relatively dense such as the proximal face of the occlusion nor a dissection flap. Instead, guidewires embed and penetrate such structures. The catheter advancement element 300 has a softness, taper, and sizing that finds and/or creates space. For example, the catheter advancement element 300 upon encountering an occlusion such as an atherosclerotic lesion or embolus can slide between a portion of the occlusion and the vessel wall rather than penetrating through it like a guidewire does. In the case of a partially occluded vessel such as a narrowing within the carotid artery, the catheter advancement element 300 can atraumatically and safely find the path through the narrowing. The catheter advancement element 300 also deflects away from a dissection flap so as to remain within the larger lumen. The softness, taper, and sizing of the catheter advancement element 300 allows for it to be repeatedly passed through the carotid and into the cerebral arteries without penetrating or taking a detour relative to these structures. The distal tip region deflects and passes by these structures so that the catheter system is advanced past them to a distal occlusion site or probes and wedges near them in a safe manner. Methods of using the catheter advancement element 300 without a guidewire or with a rescue guidewire 500 parked within the lumen 368 (see FIG. 1D) to deliver a catheter to distal regions of the brain, such as at a proximal face of occlusion, are described in more detail below.

A guidewire can extend through the single lumen 368 generally concentrically from a proximal opening to a distal opening 326 at the distal end 325 of the catheter advancement element 300 through which the guidewire can extend. In some implementations, the proximal opening is at the proximal end of the catheter advancement element 300 such that the catheter advancement element 300 is configured for over-the-wire (OTW) methodologies. In other implementations, the proximal opening is a rapid exchange opening through a wall of the catheter advancement element 300 such that the catheter advancement element 300 is configured for rapid exchange rather than or in addition to OTW. In this implementation, the proximal opening extends through the sidewall of the elongate body 360 and is located a distance away from a proximal tab or luer 364 and distal to the proximal portion 366. The proximal opening can be located a distance of about 10 cm from the distal end region 346 up to about 20 cm from the distal end region 346. In some implementations, the proximal opening can be located near a region where the elongate body 360 is joined to the proximal portion 366, for example, just distal to an end of the hypotube. In other implementations, the proximal opening is located more distally such as about 10 cm to about 18 cm from the distal-most end of the elongate body 360. A proximal opening that is located closer to the distal end region 346 allows for easier removal of the catheter advancement element 300 from the catheter 200 leaving the guidewire in place for a “rapid exchange” type of procedure. Rapid exchanges can rely on only a single person to perform the exchange. The catheter advancement element 300 can be readily substituted for another device using the same guidewire that remains in position. The single lumen 368 of the elongate body 360 can be configured to receive a guidewire in the range of 0.014″ (0.356 mm) and 0.018” (0.457 mm) diameter, or in the range of between 0.014″ and 0.022″ (0.356 mm -0.559 mm). In this implementation, the inner luminal diameter of the elongate body 360 can be between 0.020″ and 0.024″ (0.508 mm - 0.610 mm). The guidewire, the catheter advancement element 300, and the catheter 200 can all be assembled co-axially for insertion through the working lumen of the guide sheath 400. The inner diameter of the lumen 368 of the elongate body 360 can be 0.019″ to about 0.021″ (0.483 mm - 0.533 mm). The distal opening from the lumen 368 can have an inner diameter that is between about 0.018″ to about 0.024″ (0.457 mm -0.610 mm). The distal opening from the lumen 368 can have an inner diameter that is between about 0.016″ to about 0.028″ The distal opening is sized to receive a guidewire that can be a 0.014″ to a 0.024″ guidewire.

The region near the distal end region 346 can be tapered such that the outer diameter tapers over a length of about 0.5 cm to about 5 cm, or 1 cm to about 4 cm, or other length as described elsewhere herein. The larger outer diameter can be at least about 1.5 times, 2 times, 2.5 times, or about 3 times larger than the smaller outer diameter. The distal end region 346 can taper along a distance from a first outer diameter to a second outer diameter, the first outer diameter being at least 1.5 times the second outer diameter. In some implementations, the distal end region 346 tapers from about 0.080″ (2.032 mm) to about 0.031″ (0.787 mm). In some implementations, the smaller outer diameter at a distal end of the taper can be about 0.026″ (0.66 mm) up to about 0.040″ (1.016 mm) and the larger outer diameter proximal to the taper is about 0.062″ (1.575 mm) up to about 0.080″ (2.032 mm). Also, the distal end region 346 can be formed of a material having a material hardness (e.g. 62A and 35D) that transitions proximally towards increasingly harder materials having (e.g. 55D and 72D) up to the proximal portion 366. A first segment of the elongate body 360 including the distal end region 346 can be formed of a material having a material hardness of 35D and a length of about 10 cm to about 12.5 cm. The first segment of the elongate body 360 including the distal end region 346 can be formed of a material having a material hardness of 62A and a length of about 10 cm to about 12.5 cm. A second segment of the elongate body 360 can be formed of a material having a material hardness of 55D and have a length of about 5 cm to about 8 cm. A third segment of the elongate body 360 can be formed of a material having a material hardness of 72D can be about 25 cm to about 35 cm in length. The three segments combined can form an insert length of the elongate body 360 from where the proximal portion 366 couples to the elongate body 360 to the terminus of the distal end region 346 that can be about 49 cm in length.

In preferred embodiments it has been found that having a flexible distal tapered probing tip section having a length in the range of 1 cm to 5 cm and that tapers from a proximal outer diameter in the range of 1.58 mm - 2.03 mm to a distal outer diameter in the range of 0.66 mm - 0.79 mm, the atraumatic tip preferably being radiopaque, that the tapered tip region has a flexibility allowing it to deflect generally away from a dense occlusion towards the vessel wall or a pathway through an occlusion. The deflection occurs upon advancement of the catheter advancement element through the vessel on encountering a resistance to further axial motion from a generally organized or dense occlusion within a flexible vessel having an inner diameter about 2 - 5 mm for an occlusion located in the MCA or larger inner diameter up to about 8 mm for an occlusion located proximal to the MCA such as within the ICA. The tip region is arranged to deflect away from a dissection flap to find the larger pathway through the vessel. The tip region is also arranged to deflect away from a proximal face of the occlusion towards the vessel wall and, in some instances, to move at least partially under the proximal face of the occlusion so that between about 0 mm to about 3 cm of the probing tip section extends between the obstacle and the vessel wall upon application of an additional force to urge the probing tip section against the occlusion. In still other implementations, the flexible distal tapered probing tip section is configured to find a path through an occlusion (e.g., a carotid occlusion or intracranial atherosclerotic lesion) as the system is advanced to distal sites, which will be described in more detail below.

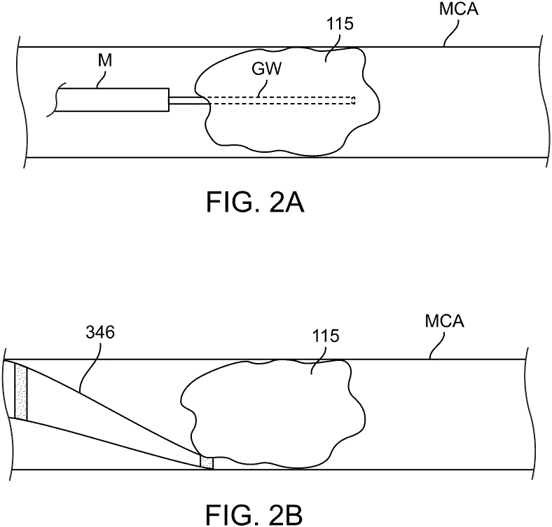

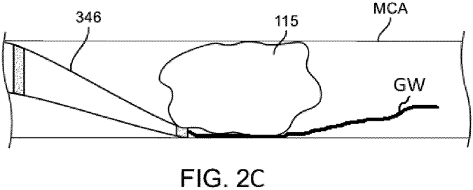

Conventional catheters and guidewires have a tip structure that tend to embed into these structures as opposed to probe them to find a space or deflect away from them. Guidewires have small outer diameters and flexible distal tips. Despite the small outer diameter and the flexibility, a guidewire tip is incapable of probing the occlusion according to the methods provided herein. Rather, a guidewire tip construction, particularly when used with a microcatheter that provides a centering effect on the guidewire, results in the guidewire penetrating and embedding into the occlusion. FIG. 2A illustrates a conventional guidewire GW extending through and centered by a microcatheter M. The guidewire GW has a tip region embedded within and penetrating an occlusion 115. FIG. 2B illustrates the tapered distal tip region 346 of a catheter advancement element probing the occlusion 115 so that the tip deflects and slips between the proximal face of the occlusion 115 and the vessel wall. This is particularly useful where the occlusion 115 is an embolus to be removed by, for example, aspiration thrombectomy. For an atherosclerotic lesion to be stented, the tip may deflect and find the narrowed path in a manner that avoids creating a new path through the lesion. FIG. 2C illustrates advancement of the tapered distal tip region 346 advancing over a pre-placed guidewire GW through an occlusion 115. The nature of the tapered tip region 346 provides atraumatic advancement of the catheter advancement element 300 even if the true lumen of the vessel were not visible or poorly visualized.

The distal end region of the guidewire has a profile that is much smaller compared to the profile of the distal tip region 346 of the catheter advancement element. The polymeric distal end region 346 can taper from a relatively small size (e.g., about 0.030″ OD, 0.019″ ID) to a relatively large size that substantially fills the catheter it extends through (e.g., 0.062″ OD for an 0.070″ ID, or 0.080″ OD for an 0.088″ ID). This taper shape and angle along with the fully polymeric, unreinforced structure allows for it to be used to gently dilate the ICAD lesion as a bougie as described elsewhere herein. The outer diameter of the guidewire also stays small moving proximally along its length compared to the catheter advancement element that enlarges to an even larger outer diameter moving proximally just a few centimeters. In turn, the force per unit area for the guidewire is much higher compared to the catheter advancement element. A guidewire used in the neurovasculature, particularly at the level of the MCA, may have an outer diameter at the distal end that is 0.014″ (0.36 mm) and have a distal-facing contact area that is about 1.50 x 10⁻⁴ square inch (0.100 mm²). The outer diameter of the distal end of the catheter advancement element can be about 0.031″ (0.79 mm) and the inner diameter of the distal end of the catheter advancement element can be about 0.021″ (0.53 mm). The distal-facing contact area for the catheter advancement element can be about 8.00 x 10⁻⁴ square inch (0.5 mm²) if the lumen is filled with a column of fluid and/or a guidewire. The distal-facing contact area for the catheter advancement element can be about 4.20 x 10⁻⁴ square inch (0.27 mm²) for just the annular distal-facing surface without a column of fluid or guidewire within the lumen. Regardless, the force per unit area of the guidewire is significantly greater (i.e., about 2 to 5 times greater) than the force per unit area of the catheter advancement element. The force per unit area of a 0.014″ guidewire for 1 N force is about 6,700 N/square inch (10 N/mm²) whereas the force per unit area of the catheter advancement element is about 1,300 N/square inch (2 N/mm²) to about 2,400 N/square inch (4 N/mm²). The profile of the guidewire, in combination with the force per unit area for the guidewire (and centering effect provided by the microcatheter), creates a higher risk of penetration of the embolus (or whatever obstruction is present within the lumen of the vessel being navigated) rather than deflection upon encountering the structure. The profile of the catheter advancement element including the greater outer diameter as the distal end, the relatively short taper to an even larger outer diameter, and its high flexibility results in the catheter advancement element being incapable of penetrating the occlusion and instead deflecting away from the proximal face of the occlusion upon encountering one within a vessel. Guidewires penetrate an embolus, atherosclerotic lesion, or vessel wall. The catheter advancement element, in contrast, probes and deflects away from the occlusion, finds any space and wedges into a final resting spot without penetrating the occlusion or the vessel wall. The catheter advancement element need not always deflect away from the occlusion between the occlusion and the vessel wall. For example, in some situations, a patient may have a partially occluded, narrowed vessel. The occluded vessel may still have a lumen extending through it, but the lumen is narrowed so that it is only 2% to 20% patent. The catheter advancement element can deflect away from the more organized portions of the occlusion and atraumatically probe the narrowed lumen through the occlusion finding the space for advancement of the catheter system.

It is desirable to have a specially constructed tip region to ensure the tip region will deflect relative to a structure such as an atherosclerotic lesion or an embolus, not penetrate the structure, when encountering it within the vessel. The tip region will deflect until it finds a path or space whether that space is merely a space between the proximal face of the occlusion and the vessel wall or a narrowed lumen through an obstruction. This is achieved by having a sufficient degree of flexibility of the fully polymeric (i.e., having no reinforcement layer) distal tip region that includes a taper over a length so that the tip region deflects readily upon coming into contact with the obstruction (e.g., proximal face of an embolus, narrowed lumen, or dissection flap). The flexibility and shape of the tapered tip region results in the tip region, which is protruding from the aspiration catheter during advancement through the vessel, passing through less organized or less dense thrombotic material until the tip region encounters a more dense structure such as the true proximal face of the occlusion. The tip region then deflects away from the organized or dense portion of the occlusion so that, for example, it wedges between the occlusion and the vessel wall or finds the lumen through it. The tip region is constructed to find the path of least resistance in an atraumatic manner without being so flexible or prone to bending that it folds over onto itself and cannot be advanced.

The distal-most tip of the tip region can have a smooth, relatively rounded shape having a low friction outer surface that tends to encourage deflection of the tip region relative to the proximal face of the occlusion. The distal tip can also be radiopaque due to embedding a material within the polymer as described in more detail below.

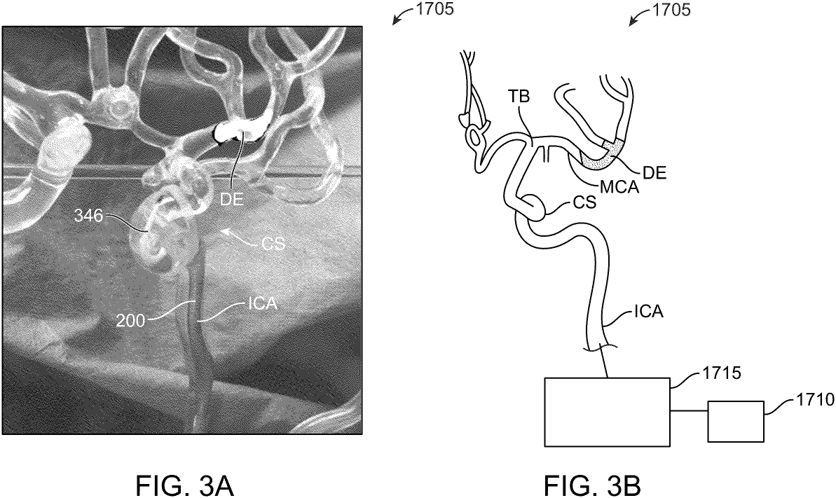

One of skill in the art can “tune” the distal tip region to have one or more properties to achieve the novel requirements set out herein. However, because the requirements are so unusual, it may be useful to measure the properties of the distal tip region using a test rig 1705. For example, FIG. 3A illustrates an implementation of a test rig 1705 and FIG. 3B is a schematic of the test rig 1705 in FIG. 3A. The test rig 1705 can include a 3D printed model of clear silicone material based on a CT/MRI scan data of an actual human patient that is configured to be connected to a pump 1710 for delivering a liquid from a source 1715 to simulate the endovascular environment. The vessels modeled by the test rig 1705 can vary, including, but not limited to femoral artery, abdominal aortic artery, renal artery, aortic artery, subclavian artery, carotid artery, and intracranial arteries. The intracranial arteries of the test rig 1705 can include various sized vessels including the internal carotid artery ICA, the carotid siphon CS, the terminal bifurcation TB of the ICA, and the middle cerebral artery MCA. A dummy embolus DE formed of a suitable material can be positioned within the vessel model, for example, within the MCA as shown in FIG. 3B, to simulate an actual embolus. The dummy embolus DE can simulate an atherosclerotic lesion that causes a narrowing of a vessel as well. The material can include a moldable, compressible polymeric material that can be compressed into a small plug shape suitable for insertion into a vessel of interest on the test rig 1705. FIGS. 3A-3B illustrate the dummy embolus DE positioned within the MCA of the test rig 1705 distal to the terminal bifurcation TB of the ICA. The larger vessels of the test rig 1705 can have an internal diameter of about 10 mm that decreases down to about 5 mm ID and towards the most narrow vessels about 2 mm inner diameter. The model vessel containing the dummy embolus DE can have an inner diameter of about 2 mm up to about 3 mm and can taper along its length although smaller or larger vessels can also be used. The dummy embolus DE can be compressed into a plug that has a maximum outer diameter that substantially matches the inner diameter of the vessel being obstructed by the dummy embolus DE. The material of the dummy embolus DE can have an outer diameter prior to being compressed that is about 6 mm to about 8 mm and a length of about 5 mm. The length of the dummy embolus DE can increase upon being compressed into the smaller diameter plug or can be trimmed after compressing to have a particular length. The dummy embolus DE once compressed can be positioned within the target vessel. The dummy embolus DE once positioned in the target vessel can fully or partially block fluid flow through the model and past the dummy embolus DE. The dummy embolus DE can have a density at its proximal face that is comparable to a typical embolus treated in this part of the cerebral vasculature and used to observe the degree of deflection a distal tip region 346 of a catheter advancement element positioned distal to the aspiration catheter 200 being advanced. The material of the dummy embolus DE can be selected so as to have different consistencies to emulate the different types of emboli that might be encountered. The test rig 1705 provides a way to assess whether the distal tip region 346 of the catheter advancement element will deflect or embed within the dummy embolus DE. The test rig 1705 can also assess the impact of a guidewire positioned within the lumen of the catheter advancement element, for example so the distal end of the guidewire is positioned proximal to the distal opening from the lumen, on the deflection of the distal tip region 346 upon encountering the different dummy emboli DE. Those of skill in the art may have alternative test rigs incorporating alternative real or synthetic embolus test subjects including other materials shaped to form an obstruction in the vessel.

The catheter advancement element 300 can incorporate a reinforcement layer. The reinforcement layer can be a braid or other type of reinforcement to improve the torqueability of the catheter advancement element 300 and help to bridge the components of the catheter advancement element 300 having such differences in flexibility. The reinforcement layer can bridge the transition from the rigid, proximal portion 366 to the flexible elongate body 360. In some implementations, the reinforcement layer can be a braid positioned between inner and outer layers of PEBAX. The reinforcement layer can terminate a distance proximal to the distal end region 346. The distal end region 346 can be formed of a material having a material hardness of at most about 35D. The first segment can be unreinforced polymer having a length of about 4 cm up to about 12.5 cm without metal reinforcement. The third segment of the elongate body 360 located proximal to the first segment can include the reinforcement layer and can extend a total of about 37 cm up to the unreinforced distal segment. A proximal end region of the reinforcement layer can overlap with a distal end region of the proximal portion 366 such that a small overlap of hypotube and reinforcement exists near the transition between the proximal portion 366 and the elongate body 360.

An entry port for a procedural guidewire can be positioned a distance away from the distal-most end of the elongate body 360. In some implementations, the entry/exit port can be about 18 cm from the distal-most end creating a rapid exchange wire entry/exit segment. The outer diameter of the elongate body 360 within the first two segments can be about 0.080″-0.082″ (2.032 mm - 2.083 mm) whereas the third segment proximal to this rapid exchange wire entry/exit segment can have a step-down in outer diameter such as about 0.062″-0.064″ (1.575 mm – 1.626 mm).

The tubular portion of the catheter advancement element 300 can have an outer diameter that has at least one snug point. A difference between the outer diameter at the snug point and the inner diameter of the lumen at the distal end of the distal, catheter portion can be no more than about 0.015″ (0.381 mm), or can be no more than about 0.010″ (0.254 mm). The at least one snug point of this tubular portion can be a point along the length of the tubular portion. The at least one snug point of this tubular portion can have a length that is at least about 5 cm up to about 50 cm, including for example, at least about 6 cm, at least about 7 cm, at least about 8 cm, at least about 9 cm, at least about 10 cm, at least about 11 cm, or at least about 12 cm up to about 50 cm. This length need not be uniform such that the length need not be snug along its entire length. For example, the snug point region can include ridges, grooves, slits, or other surface features.

In other implementations, the entire catheter advancement element 300 can be a tubular element configured to receive a guidewire through both the proximal portion 366 as well as the elongate body 360. For example, the proximal portion 366 can be a hypotube or tubular element having a lumen that communicates with the lumen 368 extending through the elongate body 360 (shown in FIG. 1C). In some implementations, the proximal portion 366 can be a skived hypotube of stainless steel coated with PTFE having an outer diameter of 0.026″ (0.660 mm). In other implementations, the outer diameter can be between 0.024″ (0.610 mm) and 0.030″ (0.762 mm). In some implementations, such as an over-the-wire version, the proximal portion 366 can be a skived hypotube coupled to a proximal hub or luer 364. The proximal portion 366 can extend eccentric or concentric to the distal luminal portion 222. The proximal portion 366 can be a stainless steel hypotube. The proximal portion 366 can be a solid metal wire that is round or oval cross-sectional shape. The proximal portion 366 can be a flattened ribbon of wire having a rectangular cross-sectional shape. The ribbon of wire can be curved into a circular, oval, c-shape, or quarter circle, or other cross-sectional shape along an arc. The proximal portion 366 can have any of variety of cross-sectional shapes whether or not a lumen extends therethrough, including a circular, oval, C-shaped, D-shape, or other shape. In some implementations, the proximal portion 366 is a hypotube having a D-shape such that an inner-facing side is flat and an outer-facing side is rounded. The rounded side of the proximal portion 366 can be shaped to engage with a correspondingly rounded inner surface of the sheath 400. The hypotube can have a lubricious coating such as PTFE or other lubricious polymer covering the hypotube. The hypotube can have an inner diameter of about 0.021″ (0.533 mm), an outer diameter of about 0.0275″ (0.699 mm), and an overall length of about 94 cm providing a working length for the catheter advancement element 300 that is about 143 cm. Including the proximal luer 364, the catheter advancement element 300 can have an overall length of about 149 cm. In some implementations, the hypotube can be a tapered part with a length of about 100 mm, starting proximal with a thickness of 0.3 mm and ending with a thickness of 0.10 mm to 0.15 mm. In still further implementations, the elongate body 360 can be a solid element coupled to the proximal portion 366 having no guidewire lumen.

The proximal portion 366 is shown in FIG. 1A as having a smaller outer diameter compared to the outer diameter of the elongate body 360. The proximal portion 366 need not step down in outer diameter and can also have the same outer diameter as the outer diameter as the elongate body 360. For example, the proximal portion 366 can incorporate a hypotube or other stiffening element that is coated by one or more layers of polymer resulting in a proximal portion 366 having substantially the same outer diameter as the elongate body 360.

At least a portion of the solid elongate body 360, such as the elongate distal end region 346, can be formed of or embedded with or attached to a malleable material that skives down to a smaller dimension at a distal end. The distal end region 346 can be shaped to a desired angle or shape similar to how a guidewire may be used. The malleable length of the elongate body 360 can be at least about 1 cm, 3 cm, 5 cm, and up to about 10 cm, 15 cm, or longer. In some implementations, the malleable length can be about 1%, 2%, 5%, 10%, 20%, 25%, 50% or more of the total length of the elongate body 360. In some implementations, the catheter advancement element 300 can have a working length of about 140 cm to about 143 cm and the elongate body 360 can have an insert length of about 49 cm. The insert length can be the PEBAX portion of the elongate body 360 that is about 49.5 cm. As such, the malleable length of the elongate body 360 can be between about 0.5 cm to about 25 cm or more. The shape change can be a function of a user manually shaping the malleable length prior to insertion or the distal end region 346 can be pre-shaped at the time of manufacturing into a particular angle or curve. Alternatively, the shape change can be a reversible and actuatable shape change such that the distal end region 346 forms the shape upon activation by a user such that the distal end region 346 can be used in a straight format until a shape change is desired by the user. The catheter advancement element 300 can also include a forming mandrel extending through the lumen of the elongate body 360 such that a physician at the time of use can mold the distal end region 346 into a desired shape. As such, the moldable distal end region 346 can be incorporated onto an elongate body 360 that has a guidewire lumen.

The elongate body 360 can extend along the entire length of the catheter 200, including the distal luminal portion 222 and the proximal extension 230 or the elongate body 360 can incorporate the proximal portion 366 that aligns generally side-by-side with the proximal extension 230 of the catheter 200. The proximal portion 366 of the elongate body 360 can be positioned co-axial with or eccentric to the elongate body 360. The proximal portion 366 of the elongate body 360 can have a lumen extending through it. Alternatively, the portion 366 can be a solid rod or ribbon having no lumen.

Again with respect to FIGS. 1A-1D, like the distal luminal portion 222 of the catheter 200, the elongate body 360 can have one or more radiopaque markers 344 along its length. The one or more markers 344 can vary in size, shape, and location. One or more markers 344 can be incorporated along one or more parts of the catheter advancement element 300, such as a tip-to-tip marker, a tip-to-taper marker, an RHV proximity marker, a Fluoro-saver marker, or other markers providing various information regarding the relative position of the catheter advancement element 300 and its components. The at least one radiopaque marker can identify the tapered end region of the elongate body 360. In some implementations and as best shown in FIGS. 1C-1D, a distal end region can have a first radiopaque marker 344a and a second radiopaque marker 344b can be located to indicate the border between the tapering of the distal end region 346 and the more proximal region of the elongate body 360 having a uniform or maximum outer diameter. It should be appreciated a single marker can identify both the distal end region and the proximal end of the taper. Identifying a proximal end of the taper provides a user with information regarding an optimal extension of the distal end region 346 relative to the distal end of the luminal portion 222 to minimize the lip at this distal end of the luminal portion 222 for advancement through tortuous anatomy. In other implementations, for example where the distal end region 346 is not necessarily tapered, but instead has a change in overall flexibility along its length, the second radiopaque marker 344b can be located to indicate the region where the relative flexibilities of the elongate body 360 (or the distal end region 346 of the elongate body 360) and the distal end of the luminal portion 222 are substantially the same. The marker material may be a platinum/iridium band, a tungsten, platinum, or tantalum-impregnated polymer, a coil, or other radiopaque marker that does not impact the flexibility of the distal end region 346 and elongate body 360. In some implementations, the radiopaque markers are extruded PEBAX loaded with tungsten for radiopacity. In some implementations, the proximal marker band can be about 2.0 mm wide and the distal marker band can be about 2.5 mm wide to provide discernable information about the distal end region 346.

The catheter 200 and catheter advancement element 300 (with or without a guidewire) can be advanced as a single unit through both turns of the carotid siphon. Both turns can be traversed in a single smooth pass or throw to a target in a cerebral vessel without the step-wise adjustment of their relative extensions and without relying on the conventional step-wise advancement technique with conventional microcatheters. The catheter 200 having the catheter advancement element 300 extending through it allows a user to advance them in unison in the same relative position from the first bend of the siphon through the second bend beyond the terminal cavernous carotid artery into the ACA and MCA. Importantly, the advancement of the two components can be performed in a single smooth movement through both bends without any change of hand position.

The catheter advancement element 300 can be in a juxtapositioned relative to the catheter 200 that provides an optimum relative extension between the two components for single smooth advancement. The catheter advancement element 300 can be positioned through the lumen of the catheter 200 such that its distal end region 346 extends just beyond a distal-most end 215 of the catheter 200. The distal end region 346 of the catheter advancement element 300 eliminates the stepped transition between the inner member and the outer catheter 200 thereby avoiding issues with catching on branching vessels within the region of the vasculature such that the catheter 200 may easily traverse the multiple angulated turns of the carotid siphon. The optimum relative extension, for example, can be the distal end region 346 of the elongate body 360 extending just distal to a distal-most end 215 of the catheter 200. A length of the distal end region 346 extending distal to the distal-most end 215 of the catheter 200 during advancement can be between 0.5 cm and about 4 cm. This juxtaposition can be a locked engagement with a mechanical element or simply by a user holding the two components together. The mechanical locking element can be a fixed or removable mechanical element 605 configured to connect to one or more of the catheter 200, the catheter advancement element 300, and the guidewire 500. The mechanical locking element 605 can be slidable along at least a length of the system components when coupled so that the mechanical attachment is adjustable. The mechanical locking element 605 can be a disposable feature or reusable for connecting to at least a portion of the shaft or a more proximal portion of the component such as the luer or hub at a proximal end of the component. In some implementations, the mechanical locking element 605 can be clamped onto the catheter 200 and the catheter advancement element 300 in a desired relative position so that the two can be advanced together without the relative position being inadvertently changed. The relative position can be changed, if desired, while the mechanical locking element 605 is clamped onto the catheter 200 and the catheter advancement element 300. The mechanical locking element 605 can be additionally clamped onto a region of the guidewire 500 extending through the catheter advancement element 300 such that the relative position of all three components can be maintained during advancement until a relative sliding motion is desired. In still further implementations, the clamping position of the mechanical locking element 605 can be changed from engaging with a first combination of components (e.g., the catheter, catheter advancement element, and the guidewire) to a different combination of components (e.g., the catheter advancement element and the guidewire) depending on what phase of the method is being performed. In still further implementations, the guidewire 500 is held fixed relative to the catheter advancement element 300 via a rotating hemostatic valve coupled to the proximal hub 434 and the catheter advancement element 300 is held fixed to the catheter 200 by a separate mechanical locking element 605. Whether the relative position of the components is fixed by a mechanical element, a combination of mechanical elements, or by a user, the proximal portions 264 of each of the catheter 200 and the catheter advancement element 300 (and the guidewire 500, if present) are configured to be held at a single point by a user. For example, where the catheter and catheter advancement element are advanced and/or withdrawn manually, the single point can be between just a forefinger and thumb of the user.

In an implementation, the mechanical locking element 605 can be a plastic spring clip configured to be flexed from a resting, “locked” configuration where the clip and the component are in clamped engagement with one another and a flexed, “unlocked” configuration where the component may be removed from engagement with the clip. The clip can be formed of unitary piece of silicone rubber or another plastic configured to be repeatedly deformed or flexed away from its set shape. The clip can be in the shape of a V having one or more channels extending along an upper surface of the valley of the V and a lower surface of the valley acting as a living hinge so that the clip channel snap fits with the component of interest (i.e., proximal portion 264 of the catheter 200, catheter advancement element 300, and/or guidewire 500). When the clip is in the resting configuration, opposing arms forming an upper end of the channel are positioned near one another so that the channel is nearly fully tubular. When the opposing arms forming the upper end of the channel are positioned near one another any component positioned within the tubular portion of the channel is enclosed within the channel and the clip “locked” onto the component. When the opposing arms of the channel are separated from one another such as upon flexing the clip around the hinge, the component can be removed from the channel. Thus, the space between the arms in the resting “locked” configuration of the clip is less than an outer diameter of the component being received in the channel and the space between the arms in the flexed “unlocked” configuration is greater than the outer diameter of the component in the channel so that the component can be withdrawn from the engagement. The clip can be flexed with a single hand due to the shape of the clip being in the form of a V. The channel extending along the upper surface can be located within a valley of the V and two wings can extend upward at an angle on either side of the valley. A user may press down on the two wings to increase the angle of the wings separating them from one another and flattening out the V shape, which in turn flexes the arms of the channel away from one another thereby opening the channel. Releasing the two wings results in their returning to the original angle and V shape, which in turn causes the arms of the channel to return to their resting position closer to one another closing the channel around the component. A user may also flip the clip over and press onto the lower surface of the clip to change the angle of the two wings, flatten out the V, and open the channel. The surfaces of the clip can be rounded and smooth to ensure comfort of the user when squeezing or pressing on the clip to flex the clip into the unlocked configuration.

The components can be advanced together with a guidewire, over a guidewire pre-positioned, or without any guidewire at all. In some implementations, the guidewire can be pre-assembled with the catheter advancement element 300 and catheter 200 such that the guidewire extends through a lumen of the catheter advancement element 300, which is loaded through a lumen of the catheter 200, all prior to insertion into the patient. The pre-assembled components can be simultaneously inserted into the sheath 400 and advanced together up through and past the turns of the carotid siphon. A guidewire may be located within the lumen 368 of the catheter advancement element 300. During advancement of the system, with reference to FIG. 2C, the guidewire GW can be first advanced to be positioned distally of the occlusion 115 or other treatment site. The catheter 200 pre-assembled with the catheter advancement element 300 can then be advanced over the pre-positioned guidewire GW to the occlusion 115. Alternatively, as shown in FIG. 2B, the system may be advanced without the guidewire GW initially placed distal of the treatment site. In this implementation, the guidewire GW can be parked proximal of the tapered distal end region 346 or proximal of the distal tip for potential use in the event the catheter advancement element without a guidewire does not reach the target location. For example, a distal tip of the guidewire 500 can be positioned about 5 cm to about 40 cm, or about 20 cm to about 30 cm proximal of the distal end region 346 of the catheter advancement element 300. At this location the guidewire does not interfere with the performance or function of the catheter advancement element. The guidewire can be positioned within the lumen of the catheter advancement element such that the distal end of the guidewire is within the catheter advancement element during the step of advancing the assembled system of devices together and is extendable from the catheter advancement element out the distal opening 326 when needed for navigation. In one example, a rescue guidewire is parked within the lumen of the catheter advancement element with a distal end of the guidewire about 0 cm to about 40 cm proximal or about 5 cm to about 35 cm proximal or about 7 cm to about 30 cm of the distal end of the catheter advancement element, preferably about 10 cm proximal of the distal end of the catheter advancement element. The guidewire at this parked position can provide additional support for the proximal portion of the system without affecting the flexibility and performance of the distal portion of the system.

FIG. 1D illustrates a rescue guidewire 500 parked within the lumen 368 of the catheter advancement element 300. In some implementations, the distal end of the guidewire 500 can be positioned inside the lumen 368 approximately flush with (0 cm) the distal opening 326 of the catheter advancement element during advancement through the vasculature. In some implementations, the distal end of the guidewire 500 can be positioned inside the lumen 368 a distance proximal from the distal opening 326 of the catheter advancement element during advancement through the vasculatures. The distance between the distal end of the parked guidewire 500 and the distal opening 326 can be at least about 1.5 cm, at least about 3 cm, at least about 5 cm, at least about 10 cm, at least about 15 cm, at least about 20 cm, at least about 25 cm, at least about 30 cm, up to about 40 cm proximal to the distal opening 326. Positioning the distal end of the parked guidewire 500 closer to the distal opening 326 of the catheter advancement element (e.g., 1.5 cm to about 3 cm proximal to the distal opening 326) such that it extends within the lumen 368 of the distal tip region 346 can support or stiffen the distal tip region 346 and also support the more proximal regions of the catheter advancement element 300. For example, a surgeon may desire to position the distal end of the guidewire closer to the distal opening 326 to increase stiffness of the distal end region of the catheter advancement element 300. Positioning the distal end of the parked guidewire 500 further away from the distal opening 326 of the catheter advancement element (e.g., greater than 5 cm up to about 40 cm proximal to the distal opening 326, preferably about 10 cm) avoids changing the flexibility characteristics of the distal tip region 346 while still supporting the more proximal regions of the catheter advancement element 300.

The guidewire can be positioned within a portion of the lumen of the catheter advancement element 300, but not extend distal to the distal opening from the lumen so that the distal-most end of the guidewire remains housed within the catheter advancement element 300 for optional use in a step of the procedure. For example, the catheter advancement element 300 having a guidewire fully contained or parked within its lumen proximal to the distal opening can be used to deliver the catheter 200 to a target location or near a target location. The catheter system advanced through the base sheath can navigate through a carotid artery while the tapered end region of the inner catheter is positioned distal to the distal end of the outer catheter and the guidewire is fully contained within the lumen of the inner catheter.

Whether or not the tapered region of the catheter advancement element 300 is advanced over the guidewire previously positioned distally or advanced together with the guidewire, the tapered end region of the inner catheter can be used during the navigating to find a passage through an occlusion in the carotid or intracranial artery. The tapered end region of the inner catheter can atraumatically probe the occlusion in the artery to find the passage, and dilate the occlusion as the taper advances through the passage prior to advancement of the catheter system. The dilation of the blockage by the advancement element 300 can allow for increased flow through the vessel and for the user to angiographically visualize the pathway of the vessel for subsequent navigation of therapeutic devices. The increased lumen created by the dilation also aids in advancement of these therapeutic devices. Additionally, the dilation of the occlusion may also allow lytic drugs to be delivered into the vessel to dissolve thrombus, in cases where there is a thrombotic component to the occlusion. In some cases, the dilation of the blockage by the catheter advancement element 300 may be enough of an increase in the lumen to negate need for further treatment with a balloon and/or stent.

In some implementations, the catheter advancement element 300 can be advanced without the catheter 200 to perform a pre-dilation step. It may be desirable to use a rapid exchange version of the inner element 300. FIGS. 12A-12B illustrate a catheter advancement element 300 having a “rapid exchange” configuration in which the lumen extends only along a distal portion of the catheter. The elongate body 360 of the catheter advancement element 300 can be generally tubular along at least a portion of its length such that a single lumen 368 extends parallel to a longitudinal axis of the catheter advancement element 300. The lumen 368 can extend only in the distal region of the elongate body 360 from the proximal opening 362 to the distal opening 326. The single lumen 368 is size-matched to accommodate a guidewire concentrically from the proximal opening 362 to the distal opening 326. The proximal opening 362 can be through a wall of the catheter advancement element and located a distance away from the distal opening 326. The proximal opening 362 can be located a distance from the distal opening 326 of about 5 cm up to about 30 cm, or about 10 cm up to about 20 cm. In some implementations, the proximal opening 362 can be located near a region where the elongate body 360 joins to a stiffer proximal portion, for example, just distal to an end of a mandrel or hypotube forming a proximal portion 366 of the catheter advancement element 300. The lumen 368, distal opening 326, and proximal opening 362 can each accommodate a guidewire having an outer diameter of about 0.014″ up to about 0.024″, or about 0.022″, or about 0.020″, or about 0.018″. For example, the inner diameter of the lumen 368 can be about 0.019” to about 0.021″ as can the diameter of the distal opening 326 and the proximal opening 362. This configuration allows the catheter advancement element 300 to be quickly exchanged for another device using the same guidewire that remains in position, such as another size of tapered catheter advancement element 300 to serially dilate the occluded site incrementally larger without the need for an exchange-length guidewire. The tapered advancement element 300 may also be exchanged for a tapered advancement element 300 assembled together with an access catheter 200 or a therapeutic device such as a balloon catheter or stent delivery device, without the need for an exchange-length guidewire.

The guidewire can be advanced distally while the catheter advancement element 300 and catheter 200 remain in a fixed position until a distal end of the guidewire is advanced beyond the distal opening 326 a distance. The catheter advancement element 300 with or without the catheter 200 can then be advanced over the guidewire that distance. The guidewire can then be withdrawn inside the lumen of the catheter advancement element 300. The guidewire can remain in position and the catheter advancement element 300 withdrawn so that another tool such as a balloon angioplasty catheter, microcatheter, or stent delivery catheter may be advanced over the pre-positioned guidewire through the catheter 200.

The tubular portion 360 of the catheter advancement element 300 can have a radiopaque marker band embedded within or positioned over a wall of the tubular portion 360 near the distal end region 346. A first radiopaque marker band 344a can be found at the distal end of the tapered distal end region 346 and a second radiopaque marker band 344b can be found at the proximal end of the tapered distal end region 346. The proximal radiopaque marker band 344b can have a proximal edge, a distal edge, and a width between the proximal and distal edges. When in the advancement configuration, the proximal edge of the radiopaque marker band 344b can align substantially with the distal end of the distal, catheter portion 222 such that the radiopaque marker band 344b remains external to the lumen 223 of the distal, catheter portion 222. At least a portion of the radiopaque marker band 344b can be positioned at the snug point, or the point of the catheter advancement element 300 where the outer diameter is no more than about 0.010″ (0.254 mm), preferably between about 0.006″ and 0.008″ (0.152 mm — 0.203 mm) smaller than the inner diameter of the catheter 200 it is positioned within. The at least one snug point of the tubular portion 360 can be located proximal to the distal end region 346 and can be where the taper of the distal end region 346 substantially ends. This allows for full extension of the tapered distal end region 346 outside the distal end of the catheter 200 and the snug point aligned substantially within the distal opening 231 from the lumen 223 of the distal, catheter portion 222 thereby minimizing any distal-facing lip that might be created by the catheter 200. The snug point can be located along at least a portion of a length of the outer diameter of the tubular portion 360 that has a length of at least about 5 cm up to about 10 cm, the outer diameter being substantially uniform or non-uniform. Additionally, the radiopaque marker bands 344a, 344b can be visible to a user without fluoroscopy, for example, prior to inserting the catheter system into the patient. The marker bands 344a, 344b can form a contrasting color visible to a user compared to a color of the polymer of the flexible elongate body, such as a black band relative to a white color of the polymer. The marker bands 344a, 344b can be useful in achieving a particular relative extension of the catheter advancement element 300 to the catheter 200 prior to insertion of the devices into an RHV. In cases where the catheter advancement element 300 is used to “pre-dilate” an occlusion, the radiopaque taper marker(s) are used to safely advance the element 300 through the occlusion. For example, visualization of the entire taper, wherein with a single radiopaque marker identifying the entire taper or radiopaque marker pairs identifying the start and end of the taper, allows for a user to device how much of the tapered tip region advances through the occlusion thereby controlling the dilation achieved by the length of the taper advanced into it.

The use of the catheter advancement element 300 with the tapered distal end region 346 allows for delivery of large bore aspiration catheters, even full-length “over-the-wire” catheters or catheters such as those described herein having a proximal extension. The catheter advancement element 300 can include a pair of radiopaque markers 344a, 344b configured to aid the operator in delivery of the system. The distal marker 344a near the distal-most end 325 of the catheter advancement element 300 can be differentiated from the distal marker 224a on the catheter 200 by its characteristic appearance under fluoroscopy as well as by simply jogging back and forth the atraumatic catheter advancement element 300 to understand the relationship and positioning of the catheter advancement element 300 relative to the catheter 200. The second marker 344b on the catheter advancement element 300 that is proximal to the distal-most tip marker 344a can delineate the taper of the distal end region 346, i.e. where the outer diameter of the catheter advancement element 300 has a sufficient size to reduce the “lip” of the transition between the catheter advancement element 300 and the catheter 200 through which it is inserted and configured to deliver. The markers aid in positioning the catheter advancement element 300 relative to the distal end 215 of the aspiration catheter 200 such that the tip 215 of the catheter 200 is aligned with the taper of the catheter advancement element 300 and the best alignment is facilitated.

The relationship between the distal tip marker 224 of the aspiration catheter 200 is at or ideally just proximal to the taper marker 344b of the catheter advancement element 300 (i.e. the proximal marker identifying the start of the taper) is identifiable with the tandem marker system. The paired elements 224, 344b are in a “tip-to-taper” position. The relative extension between the catheter advancement element 300 and the catheter 200 can be adjusted at the insertion of the system into the RHV. However, the relative extension can become altered with advancement through the sheath or guide catheter. As the system exits the guide catheter, the aspiration catheter 200 and the catheter advancement element 300 can be adjusted to that the tip-to-taper position is assumed as the system traverses the often tortuous proximal vessel (e.g. the cervical internal carotid artery) towards more distal targets. The system of the aspiration catheter 200 and the catheter advancement element 300 can be locked into their relative extension so that the juxtaposition of the catheter advancement element 300 and the aspiration catheter 200 is maintained. As the aspiration catheter 200 is visualized within the sheath distal end or even slightly beyond the distal end of the sheath, the catheter advancement element 300 can be adjusted to assume the proper position relative to the catheter before advancement resumes. The optimum relative extension between the distal marker 224 of the catheter 200 to the taper marker 344b on the catheter advancement element 300 can be maintained through as much of the anatomy as possible to maximize the delivery capability of the catheter advancement element 300 to navigate both tortuosity and to avoid side branches such as the ophthalmic artery. Once a desired site is reached, the catheter advancement element 300 can be held fixed and the aspiration catheter 200 advanced over the catheter advancement element 300 towards the occlusion 115. In some implementations, the catheter 200 is advanced without crossing the occlusion 115.

The catheter advancement element 300 is designed specifically such that the catheter 200 can be delivered without a need for a guidewire. This ability to deliver the catheter 200 without a guidewire (or with a guidewire located within the lumen 368 of the catheter advancement element 300 and parked proximal of the tapered distal end region 346 and/or proximal of the distal opening 326 for potential use) and without crossing the occlusion is based, in part, upon the smooth transitions between the outer diameter of the catheter advancement element 300 and the catheter 200 as well as the smooth transition in flexibility between the two. When the catheter advancement element 300 is bent into an arc of greater than 180 degrees, the softness and flexibility creates a smooth arc without severe bends or kinks in the geometry of the catheter. Thus, the catheter advancement element 300 seeks the larger lumens and goes where the majority of blood flow goes as opposed to into the smaller branch arteries. The distal end region 346 of the catheter advancement element 300 can facilitate a strong preference to seek out the larger vessels during advancement into the distal vessels. This propensity to stay within the main channel allows for the advancement of large bore catheters without the aid of a guidewire. The propensity to follow the main channels of blood flow aligns with acute ischemic stroke pathophysiology where major emboli tend to follow these same routes to a point where the occlusion lodges and interrupts antegrade blood flow. As well, these major channels are often ideal for placement of access catheters as these conduit arteries allow for smaller catheters to pass into specific target arteries for therapeutic intervention.

Standard neurovascular intervention, and nearly all endovascular intervention, is predicated on the concept that a guidewire leads a catheter to a target location. The guidewires are typically pre-shaped and often find side-branches of off-target locations where the guidewire will bunch or prolapse causing time-consuming nuisances during interventions that often require repeated redirection of the guidewire by the operator to overcome. In addition, this propensity of a guidewire to enter side-branches can be dangerous. Guidewires are typically 0.014″ to 0.018″ (0.356 mm – 0.457 mm) in the neuroanatomy and will find and often traumatize dissection flaps or small branches that accommodate this size, which can lead to small bleeds or dissections and further occlusion. In a sensitive area like the brain these events can be catastrophic. The tendency of a guidewire to bunch and prolapse can also cause a leading edge to the guidewire that can be advanced on its own or as part of a triaxial system to create dissection planes and traumatize small vessels. Guidewires are also designed to cross structures such as an embolus or atherosclerotic lesion, primarily for the purpose of securing the guidewire to provide support for delivery of a catheter over the guidewire. However, crossing the embolus or lesion with the guidewire can increase a risk of dislodging embolic debris, which travels distal to the occlusion site creating additional occlusion sites. Guidewires also increase the risk of perforations.

In contrast, the catheter advancement element 300 described herein preferentially stays in the larger lumen of a conduit vessel. The catheter advancement element 300 delivers to the largest lumen within the anatomy even in light of the highly tortuous anatomy and curves being navigated. The catheter advancement element 300 can preferentially take the larger lumen at a bifurcation or dissection flap while also following the current of the greatest blood flow thereby maintaining the general direction and angulations of the parent vessel. In viewing the standard anatomy found in the cerebral vasculature, the Circle of Willis is fed by two vertebral and two carotid conduit arteries. As these four arteries are the access points to the cerebral anatomy – the course of the catheter advancement element 300 can be identified and has been validated in standard cerebral anatomy models.

In the anterior circulation where the conduit artery point of entry for cerebral endovascular procedures is the internal carotid artery (ICA), the catheter advancement element can guide the large-bore catheter to the M1 segment of the middle cerebral artery (MCA) bypassing the anterior communicating artery (ACA) and anterior temporal branch (ATB). The very flexible nature of the catheter advancement element 300 combined with the distal flexible nature of most cerebral catheters combine to allow delivery through severe tortuosity. Independent of the tortuous nature of the course of the arteries, the catheter advancement element 300 tends to navigate the turns and deliver to the largest offspring from a parent artery, for example, ICA to M1 segment of the MCA. The M2 level branching of the M1 can be variable, but is often seen to have two major M2 branches (superior and inferior) and, depending on the anatomy, which can vary significantly between patients, may be seen to bifurcate “equally” or “unequally.” If the caliber of the M2 branching is of similar size and angulation, the catheter advancement element 300 may take one of the two branches. If the target for catheter placement is not in a favorable angulation or size of artery, the catheter advancement element 300 may be curved (e.g. via shaping of a malleable distal tip) and directed or a guidewire may be used.

In some anatomies where the M2 bifurcation is “even” in size, a back-and-forth motion may aid in selecting one branch then the other while still avoid the need or use of a guidewire or a curved distal tip of the catheter advancement element. The back-and-forth motion can allow for the catheter advancement element to be directed into either branch of the M2. The catheter advancement element, even when initially straight, achieves some curvature that aids in directing it into a branch vessel. Thus, when an operator encounters an M2 bifurcation and there is a desire to cannulate either branch of an evenly divided bifurcation, selection of either branch is possible using the catheter advancement element without a guidewire.

Thus, main channels such as the ICA, the middle cerebral artery and its tributaries in the anterior circulation will naturally be the pathway of preference for the described catheter advancement element and subsequence large-bore catheter delivery (via access from the ICA). A similar phenomenon can occur in the posterior circulation, which is accessed via the vertebral arteries arising from the subclavian arteries on the right and the left. The catheter advancement element will take the main channels in this circulation as well by traversing the vertebral arteries to the basilar artery and to the major tributaries of the basilar: the posterior cerebral artery and superior cerebellar arteries in the posterior circulation.

Navigation using the catheter advancement element can provide maximal deliverability with minimal vascular trauma. Catheters can cause “razoring” effects in a curved vessel because the blunt end of a large bore catheter can tend to take the greater curve in rounding a vessel when pushed by the operator. This blunt end can gouge or “razor” the greater curve with its sharp edge increasing the risk for dissection along an anatomic plane within the multilayered mid- or large-sized artery or vein (see, e.g. Catheter Cardiovasc. Interv. 2014 Feb; 83(2):211-20). The catheter advancement element can serve to minimize the edge of these catheters. Positioning the catheter advancement element within the lumen of the large-bore catheter such that the taper marker of the catheter advancement element is aligned optimally with the distal tip marker of the catheter minimizes the edge and thereby eliminates “razoring” as the large-bore catheter is advanced through turns of the vessel. This is particularly useful for the cerebral anatomy. ICAD treatments are typically needed in regions distal to the carotid siphon, particularly distal to the ophthalmic artery takeoff from the greater curve of the severe tortuosity of the final turn of the carotid siphon “S-turn”, the “anterior genu” of the carotid siphon typically seen as part of the terminal internal carotid artery (ICA). The specifics of the catheter advancement element in proper alignment within the large bore catheter (the “tip-to-taper” position noted by the distal tip marker) relative to the taper marker of the catheter advancement element maximize the likelihood that razoring and hang-up on the ophthalmic artery are avoided during manual advancement of the catheter system. The taper marker of the catheter advancement element can be positioned at or past the take-off of the ophthalmic artery to minimize these deleterious effects and allows the large-bore catheter to pass the ophthalmic artery without incident. In a relatively straight segment, which is common after passing the siphon, the large-bore catheter can be advanced over the catheter advancement element, which serves still as a guiding element to the target. The transition between the catheter advancement element and the distal edge of the large-bore catheter is insignificant, especially compared to the step changes present with a typical microcatheter or guidewire, which do not prevent hang-ups on branches such as the ophthalmic artery. The catheter advancement element allows for maneuvering of the large-bore catheter clear to the face of the occlusion without use of a microcatheter or guidewire and without crossing and/or fragmenting the occlusion in any way.

Conventional techniques to treat occlusions whether with a stent retriever, aspiration techniques, or a combination of the techniques, or to deploy a stent on an atherosclerotic lesion involve crossing the target occlusion with a guidewire. Crossing of the occlusion with a guidewire can create fragmentation of the occlusion, which can be friable and thrombotic in nature creating particulate that can be released downstream. The techniques described herein allow for the occlusion to be treated without any crossing of the occlusion with a guidewire, which tend to create their own paths through an occlusion. The systems described herein need not incorporate a guidewire. And, if a guidewire is used, it need not be advanced independently (i.e., unsheathed) to cross the target occlusion. Thus, the systems described herein can incorporate relatively large bore catheters that are delivered without disturbing the target occlusion with a guidewire, reducing the risk for stroke and downstream effects from fragmentation of the occlusion, and having improved efficiency. Additionally, the systems described herein are single-operator systems allowing the operator to work at a single RHV and, in the case of spined components, can manipulate all the elements being used to navigate the anatomy with single-handed “pinches.” This can be referred to as “monopoint.”

The catheter advancement element allows for safer and more efficient delivery of large-bore catheters to distal sites of the cerebral arteries. Large-bore catheters are particularly useful for removing thrombotic material via aspiration, or for the prevention of embolic particles to flow downstream during placement of endovascular devices such as stents across atherosclerotic lesions. Catheter inner diameter can be maximized for treating these locations to obtain more beneficial fluid dynamics for aspirating and removing thrombotic material. The safe and efficient delivery of the large bore catheter made possible by the catheter advancement element may also act as support catheters for the delivery of working devices such as stents and flow diverters with larger sizes than that possible with current cerebral catheters. For example, carotid artery stents, which can be as large as 10-11 mm, require correspondingly large stent delivery devices. Carotid stents such as the WALLSTENT (Boston Scientific) or PRECISE (Cordis) can be delivered to the neurovasculature, but their large size makes delivery particularly challenging. The catheter advancement element safely and efficiently delivers a large bore catheter (e.g., 0.070″ up to about 0.102″ ID, preferably about 0.088″) directly to the occlusion site that can then be used as a support catheter for large stent delivery systems (with or without aspiration). The catheter advancement element reduces the risk of wall perforation, particularly compared to a microcatheter-centered guidewire, so that the large bore catheter having a size that approaches the size of the vessel being treated can be delivered more safely. Other devices such as flow diverters may be optimized if delivered on larger delivery systems than currently available systems.

The distal end region 346 of the catheter advancement element 300 can be tapered, soft and flexible so that it can be used to locate a desired location – even one past the angiographic limit of contrast – for application of aspiration by the aspiration catheter 200. The softness, tapering, and sizing of the catheter advancement element 300 distal end region 346 allows for the distal most end of the distal end region 346 to pass through the soft clot material and probe the occlusion 115 without crossing the occlusion. In some implementations, the distal end region 346 can find and/or create space in or beside the occlusion 115 or slide between at least a portion of the occlusion 115 and the vessel wall. The catheter advancement element 300 can be advanced to position the distal-most end 325 of the catheter advancement element 300 without crossing the occlusion 115. Unlike a guidewire, the catheter advancement element 300 is unlikely to cross the occlusion 115 due to the extremely flexible distal tip region and the tapered walls of the distal tapered region 346. Instead the tapered distal region 346 finds a natural resting point or stopping point where further advancement is prevented or difficult. If the tapered distal end region 346 of the catheter advancement element is advanced beyond this natural stopping point and further advancing pressure is applied, the catheter advancement element can begin to buckle and/or prolapse giving the feedback that the desired advancement has been achieved. If this buckling is between the markers 344a and 344b the buckling can be seen angiographically as the marker 344b moving distally without corresponding motion of the marker 344a. Alternatively, the contact between the tapered distal end region 346 and the occlusion or dense clot material can provide feedback, for example, tactile feedback to a user handling the tools manually, that the natural resting place has been reached. If the user attempts to advance the tapered distal end region 346 of the catheter advancement element 300 beyond the natural stopping point this can result in traumatizing or fragmenting of the occlusion.

“Crossing the occlusion” or “crossing the embolus” as used herein is means that at least some portion of the device crosses to a downstream or distal side of the occlusion or embolus relative to the site of insertion. The limits of an occlusion or embolus can be difficult to assess. Thus, crossing the occlusion or the embolus includes at least some portion of the device passes an enlarged region of the occlusion or embolus even though that portion may not be fully distal to or downstream of the full distal limit of the lesion. Crossing increases the risk of embolic material being knocked loose from the occlusion or embolus and traveling downstream to create new occlusion sites. The catheter advancement element 300 can be advanced as far as possible without buckling of the catheter advancement element 300. Instead of crossing the occlusion, the catheter advancement element can interrogate the treatment site to locate a proximal face of the occlusion 115 while maintaining structural integrity of both the catheter advancement element 300 and the occlusion 115. In some instances, the tapered distal end region 346 of the catheter advancement element 300 can be used to dissect past or separate the soft clot material accumulated at the proximal face of the occlusion 115 and to probe the denser material of the occlusion 115.

In some implementations, the catheter advancement element 300 can be fixed by a user to remain in this position and the catheter 200 advanced over it to the treatment site, for example so the two components are tip-to-tip and distal markers 344a, 224a aligned. In some implementations, the catheter 200 can be advanced until resistance is felt by a user indicating the distal end 215 is positioned at the proximal face of the embolus 115. The catheter 200 can be advanced so that the distal end 215 of the catheter 200 is urged against the proximal face of the embolus 115 slightly compressing the embolus 115. The catheter 200 can be positioned so that the distal end 215 of the catheter 200 is located past the proximal face of the embolus 115, but without crossing the embolus 115. Once the catheter 200 is positioned at the treatment site at one of the locations described above, the catheter advancement element 300 can be withdrawn.

In other implementations, the catheter 200 can be advanced to seat with the embolus 115 as the catheter advancement element 300 is withdrawn. In this method, the catheter 200 can be advanced using the catheter advancement element 300 for navigation to a location that is a distance away from the proximal face of the embolus 115. The distal catheter portion 222 can become compressed during advancement through the tortuous anatomy. As the catheter advancement element 300 is withdrawn a distance relative to the distal end of the catheter 200, stored energy or compressive forces within the catheter system get released causing the distal catheter portion 222 to move distally. The catheter 200 can be allowed to ride the forward momentum as the forces are released moving the distal end of the catheter towards the embolus 115.

The forward catheter movement during removal of the catheter advancement element 300 can be supplemented by user applied force (manually or automatically) and facilitated by the internal vacuum generated by the withdrawal of the occlusive catheter advancement element 300 and the piston arrangement or “plunger” effect described elsewhere herein. Withdrawal of the catheter advancement element can simultaneously create distal motion of the catheter due to release of stored forces and internal vacuum within the catheter. The internal vacuum can, in turn, cause more distal motion of the catheter. Thus, the distal motion of the catheter can be due to both the catheter passively riding the momentum of the stored forces, and also an active drawing of the catheter towards the embolus due to the internal vacuum. The catheter advancement element can be used to deliver the catheter to a first position and the catheter allowed to nest with the target embolus located beyond this position and without the presence of the catheter advancement element (or guidewire) by virtue of the distal motion and internal vacuum created upon removal of the catheter advancement element. Thus, the catheter advancement element functions not only to deliver the catheter to a distal location near the embolus more safely than a guidewire, but also to automatically trigger or actuate forward motion of and suction through the catheter when it is withdrawn to more optimally seat the catheter with the embolus.

### Intracranial Stents and Delivery Systems

An access system with a larger inner lumen allows for a wider range of intracranial stent designs to be delivered to a treatment site for the treatment of a stenosis. Conventional access systems and methods do not allow an optimal intracranial stent design.

Neurovascular self-expanding stents such as the NEUROFORM ATLAS stent (Stryker) or ENTERPRISE Stent (Johnson & Johnson) are available for supporting embolic coil treatment of cerebral aneurysms. These stents are delivered through microcatheters and have been evaluated for use in treating intracranial stenoses. These stents, however, are not ideal because they lack the radial force needed to adequately treat intracranial stenosis. The number, width, and thickness of the stent struts are typically increased to significantly improve radial force. However, such stents cannot be compressed into very small diameters needed to be able to be delivered through a microcatheter.

Another stent called the WINGSPAN (Stryker) has been used for treating intracranial atherosclerosis and has greater radial force than the intracranial aneurysm stents described above, but their delivery systems are bulky and stiff, and require exchange-length guidewires for delivery.

More recently, balloon-expandable coronary stents, for example, the RX DRIVER Stent (Medtronic) or VISION Stent (Abbot Vascular) have been used to treat intracranial stenoses. These stents provide greater radial force than the current neurovascular devices, however, such coronary stents typically have shorter length delivery systems, and therefore are not deliverable through conventional neurovascular access systems.

The distal access system 100 described herein addresses these issues.

Balloon-expandable stents exert a force on the vessel during expansion, which in the intracranial and cerebral vessels may lead to severe complications, such as vessel dissection or injury, or blockage of important perforator vessels. Furthermore, balloon-expandable stents are by their nature plastically deformed and formed into the expanded state by the balloon. Because the balloon used to expand balloon-expandable stents is straight, these stents tend to straighten the vessels within which they are planted. This characteristic is not ideal to treat the extreme tortuosity of intracranial vessels. Thus, in many cases, self-expanding stents are preferred to balloon-expandable stents.

Self-expanding stents have greater conformability and lower radial force than balloon-expandable stents. The larger bore access systems described herein enables delivery of self-expanding devices with higher radial force than the current microcatheter-delivered neurovascular stents.

In an implementation, an endovascular scaffolding device is an intracranial stent that is constructed with thicker walls, wider struts, and/or greater density of struts than the conventional neurovascular self-expanding stents to achieve a greater radial force while maintaining greater conformability to curved vessels compared to balloon-expandable stents. The intracranial stent can be a drug-eluting stent, with a coating that contains therapeutic agent designed to reduce the risk and extent of restenosis. Examples of drug-eluting technology are well-known for coronary stents and can be applied to neurovascular implants for similar benefit.

### Flow Diverters

An access system with a larger inner lumen will allow a wider range of flow diverter designs to be delivered to an aneurysm site. Currently known flow diverters are delivered on an inner delivery core through a microcatheter with 0.027″ ID (0.7 mm). Conventional flow diverters in order to be delivered through such a delivery system size and to achieve the desired wall coverage (approximately 30%) when expanded in vessel up to 5.0 mm diameter have braided wire construction.

The delivery of conventional braided flow diverters typically occurs over several procedural steps. First, a microcatheter is inserted into the vasculature and advanced over a guidewire to a position across the target aneurysm site. The microcatheter tip is often placed far distal to the ultimate target implant site because of the imprecise nature of delivering braid-style flow diverters. The flow diverter is delivered through the microcatheter partially deployed and then “dragged back” into place across the target site. Both the distal positioning of the microcatheter and the “drag back step” are areas of risk for vessel damage and vessel perforation, both leading to severe clinical sequelae.

More specifically, the multiple procedure steps for deployment of a braided flow diverter include placing the guidewire and then placing the microcatheter across the aneurysm. Once the microcatheter is in position relative to the target aneurysm, the guidewire is removed. The braided flow diverter is then inserted to the proximal end of the microcatheter using an introducer tube. The flow diverter is mounted on a delivery core wire with features to keep the flow diverter both restrained in the collapsed configuration and secured longitudinally onto the delivery core wire. For example, the core wire can have PTFE sleeves that cover and constrain the braided flow diverter at either end. The core wire often has a distal flexible tip that extends up to 15 mm beyond the distal end of the flow diverter. This means that the distal tip needs to be positioned at least 15 mm beyond the treatment site, and possibly more if the microcatheter is positioned distally, for the flow diverter to be implanted in the correct location, another source of potential complication. The core wire is used to push the flow diverter to the end of the microcatheter. The microcatheter is then retracted to expose the braid, which, by its material properties and construction, begins to spring open. The distal end does not reach its full opening diameter until several millimeters of the braid are exposed due to the nature of the braided construction. The user must often push on the microcatheter while pulling on the core to “push” the braid to its maximum opening in order to get full apposition of the flow diverter against the vessel wall, which is highly desirable to achieve the intended clinical effect. This push and pull technique is yet another potential cause of clinical complication of conventional braided flow diverters as well as adding time to the procedure and imprecision in the implantation location. Braids by their nature shorten considerably upon expansion, making accurate implantation yet more difficult. In many flow diverter delivery systems, the delivery core wire has features that constrain the braid wire ends. The microcatheter following expansion of the flow diverter is fully proximal to the implant and must be re-advanced through the braid to cover the delivery core wire features so that the delivery core wire does not get snagged by the just-deployed flow diverter. Each of these steps potentially disrupt the flow diverter, add to procedural time, and are potential causes of clinical complications due to the extra catheter maneuvering.

Disclosed herein are laser-cut tube-style flow diverter implants that greatly improve the deployment and performance of the flow diverter. Unlike a braided wire tube, a laser cut tube expands to full diameter much more easily and precisely, shortens only minimally if at all, and does not require restraining features when constrained. Implantation of laser-cut, tube-style flow diverters is faster, safer, and more precise.

The flow diverter implants described herein are laser-cut, self-expanding Nitinol tubes having a starting outer diameter that is greater than 1 mm, capable of expanding to about 5 mm while still achieving 30-35% metal coverage even after loss of material from laser cutting and electropolishing. For example, the flow diverter implants described herein are constructed from a tube having an outer diameter of 2.25 mm to 2.50 mm that are expandable to about 5 mm diameter having 25-35% metal coverage. Such implants based on laser-cut Nitinol tube construction are very precise and quick in delivery. There is no need to constrain the distal end of the implant because the construction lacks wires. The laser-cut flow diverter implants described herein also do not experience significant foreshortening as braided wire scaffolds do. The laser-cut pattern can be designed to achieve wall apposition and coverage sufficient to achieve flow diversion, prevention or reduction of blood flow into the aneurysm, and/or isolation of the aneurysm, resulting in an inner surface that is smoother and less thrombogenic. The laser-cut implant designs described herein can vary strength and % coverage along their length for optimal performance.

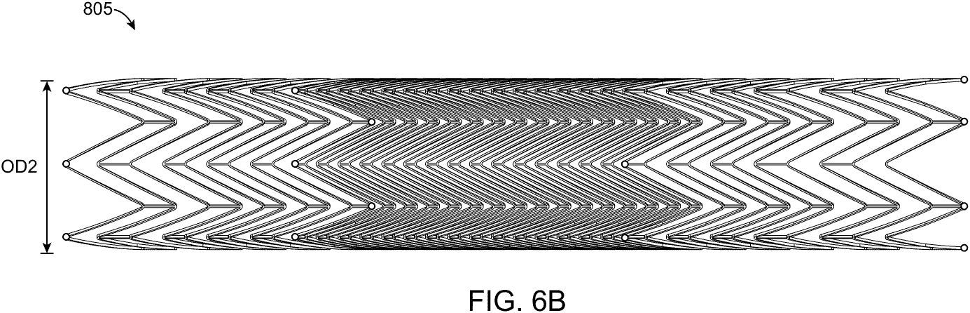

FIG. 6A shows a flow diverter 805 in the collapsed configuration having a first outer diameter OD1 suitable for delivery and FIG. 6B shows the flow diverter 805 in the expanded configuration having an enlarged second outer diameter OD2. The flow diverter 805 can be a Nitinol tube that is laser-cut and electropolished to have 30% (+/- 5%) material coverage when expanded in vessel up to 5 mm. The OD1 of the tube in the collapsed configuration can be about 2.0 mm to about 3.0 mm, with the actual OD dependent on the density of the original laser cut pattern and how much the flow diverter can be crimped down in a flow diverter delivery system. A laser cut tube can be crimped down to a size smaller than the initial tube size. A restraining sleeve of the flow diverter delivery system or delivery catheter lumen can have an ID of less than about 2.0 mm to about 3.0 mm. The flow diverter delivery system restraining sleeve or delivery catheter can have an ID of about 1.8 mm to about 2.8 mm and an OD of about 2.0 mm to about 3.0 mm. An access catheter to deliver the flow diverter delivery system having these dimensions has an inner diameter of approximately 2.2 mm (0.087″) to about 3.2 mm (0.126″). The OD2 of the tube in the expanded configuration can be about 5.0 mm.

The figures are intended to be illustrative to these dimensions including metal coverage percentages. They are not to scale in absolute terms or comparatively.

Larger access systems allow for alternate delivery methodologies. For example, rather than first placing the microcatheter across the aneurysm, removing guidewire, and then pushing the flow diverter into place as with conventional flow diverter delivery systems, the flow diverters 805 described herein can be pre-mounted onto a delivery system and delivered to the site through a larger delivery system (e.g., 0.087″ – 0.126″ ID). The guidewire 500, flow diverter 805, and delivery system can all be pre-mounted in one system rather than exchanging the guidewire 500 for the flow diverter 805 and delivery system as in conventional deliveries. The endovascular implant and flow diverter delivery systems will be described in more detail below.

The access system can be a distal access catheter that is placed using known techniques to the implant site. The distal access system can be the monopoint system shown in FIGS. 1A – 1B. The distal catheter 200 can also serve as the restraining sleeve 810 of the flow diverter delivery system.

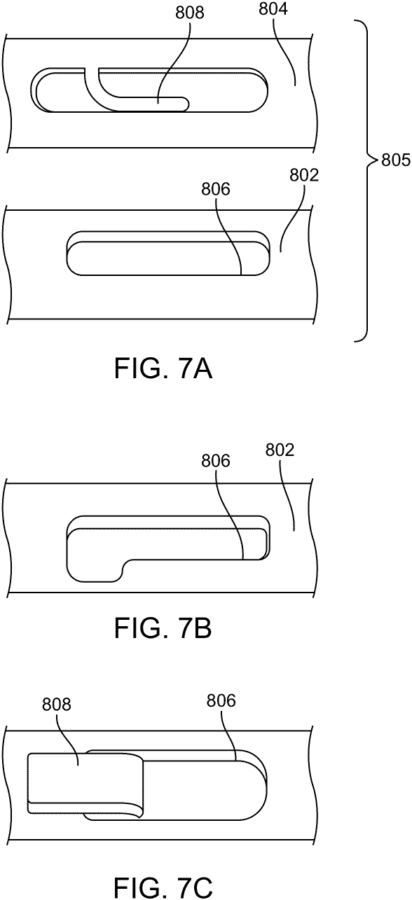

In some implementations, the flow diverter 805 is constructed from two laser-cut tubes 802, 804, which, in combination, provide approximately 30% material coverage when expanded in the vessel (see FIGS. 7A-7C). Each of the two tubes 802, 804 may be dual layer and staggered relative to one another. Staggering the two tubes 802, 804 creates an overlap region having a first density (e.g., 30% material coverage) and each end of the staggered tubes 802, 804 having a second lower density (e.g., 15% material coverage). The two tubes 802, 804 may be locked together with locking features built into the laser cut pattern. For example, as shown in FIG. 7A, one tube 802 may have one or more holes or elongate slots 806 laser cut into the tube 802 on either or both ends, and the second tube 804 may have one or more corresponding tabs 808 formed to protrude into the slot 806 and then lie flat. The two tubes 802, 804 are assembled such that the tabs 808 are inserted into the slots 806 and then the tubes 802, 804 are slid with respect to each other to lock the two tubes 802, 804 together. In a variation, as seen in FIG. 7B, the slot 806 may have an ‘L’ shape such that the two tubes 802, 804 can be rotated with respect to each other to lock the two tubes 802, 804 together. Alternately the tab 808 can be pushed through the slot 806 and bent to lock into place, as shown in FIG. 7C. The tabs 808 can be on the inner tube 804 and the slots 806 on the outer tube 802, or vice versa.

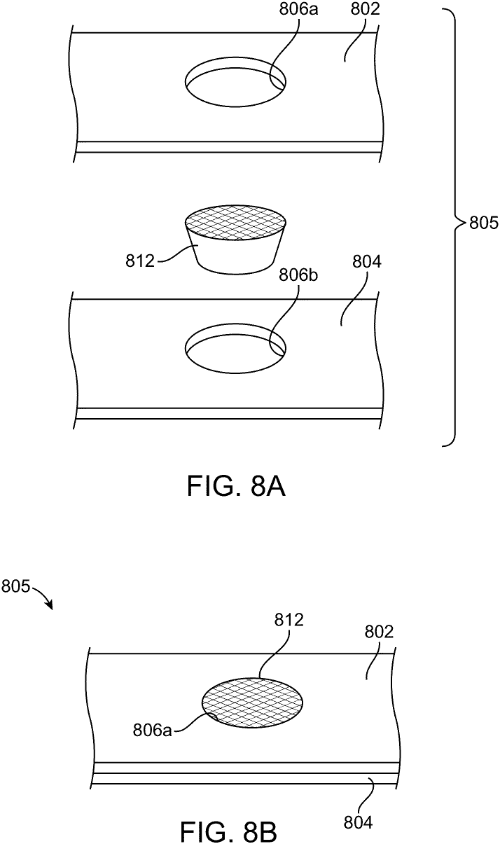

FIGS. 8A-8B illustrate another locking mechanism for a flow diverter 805 constructed from two laser-cut tubes 802, 804. Both tubes 802, 804 can be laser cut to include holes 806 or elongate slots on either or both ends in corresponding positions. The two tubes 802, 804 are assembled one inside the other so that their respective holes 806a, 806b are aligned. A disk 812 made from a malleable material can be pressed into the holes 806a, 806b to lock the tubes 802, 804 together. The disk 812 may be slightly tapered (i.e., from an upper side toward the lower side as shown in FIG. 8A) and sized such that when the disk 812 is pressed into place, the disk 812 deforms to fill the holes 806a, 806b and is held securely in place. The disk 812 can be a radiopaque malleable material such as gold, gold alloy, or tungsten to serve both as a radiopaque marker on the implant as well as a locking mechanism.

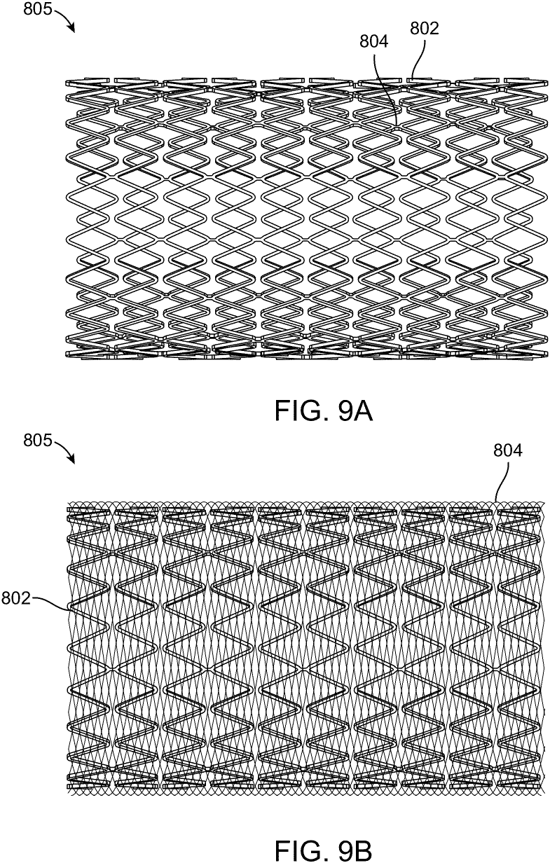

FIG. 9A illustrates another implementation of a flow diverter 805 constructed from two laser-cut tubes 802, 804. One tube 802 is designed to provide structural integrity to the flow diverter 805, for example, to provide full wall apposition and anchoring such as by a wall thickness and/or strut width. The other laser cut tube 804 is designed to provide the 30% material coverage and has a very fine strut pattern and thin wall thickness. The finer cut tube 804 may also be a very fine-wire braided tube, or a porous material such as an expanded PTFE tube, as seen in FIG. 9B. The finer strut pattern tube 804 may be shorter than the larger strut structural tube 802. Alternately, as shown in FIG. 9B, the two tubes 802, 804 may be the same length and the tubes 802, 804 substantially overlap each other.

These multi-layer flow diverter implants utilize the structural stent layer 802 to provide precise placement and anchoring, and the finer stent layer 804 to provide the higher material coverage that diverts the blood from flowing into the excluded aneurysm. The larger-diameter access systems described herein enable delivery of these multi-layer devices, which would not be possible in the current microcatheter delivery methods having smaller inner diameters (e.g., 0.027″), which as described above, are incapable of accommodating a laser-cut flow diverter alone and certainly not a flower diverter plus a restraining sleeve.

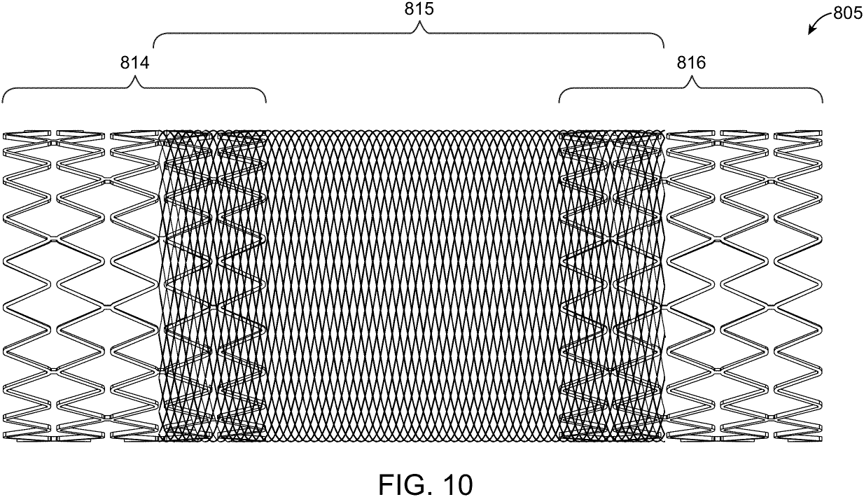

The flow diverter 805 can also be made of varying materials and structures along its length. For example, as shown in FIG. 10, the flow diverter 805 is formed of two laser cut bands 814, 816 on both ends of the device. A finer structure such as a braided wire tube 815 can be positioned between the two laser-cut bands 812, 814. The braided wire tube 815 can be interlaced with the laser-cut bands 812, 814 to couple to the bands. This compound or hybrid design provides two end anchors to the flow diverter 805 with the higher material coverage across the aneurysm.

Any of the laser-cut tube components in the flow diverters described above may be self-expanding, manufactured from one or more Nitinol laser-cut tubes. Alternately, any of the above flow diverter implants may be a balloon-mounted laser cut stents, manufactured from one or more laser-cut stainless steel, cobalt-chromium alloy, or other materials known to be used for balloon-expandable stents.

The flow diverter implant may have specialized antithrombotic surface modifications or coatings, for example, heparin coatings, hydrophilic polymer coatings such as phosphorylcholine and phenox hydrophilic polymers, albumin, fibrin, and the like.

As described elsewhere herein, the inner diameter of the distal luminal portion 222 of catheter 200 can vary between about 0.070″ – 0.102″ (1.778 mm – 2.591 mm) including 0.070″ (1.778 mm), 0.072″ (1.829 mm), 0.081″ (2.057 mm), 0.088″ (2.235 mm), 0.092″ (2.337 mm), or 0.102″ (2.591 mm). The large bore inner diameters are particularly useful for delivery of the flow diverters and flow diverter delivery systems described herein. It should be appreciated, however, that the inner diameter of the catheter 200 can have a smaller inner diameter that is less than 0.070″ (1.778 mm) including 0.069″ (1.753 mm) down to about 0.054″ (1.372 mm).

The expandable implants described herein including the flow diverters and also the stents, can have outer diameters that vary depending on their stage of deployment. The expandable implants can have an “as-cut” outer diameter, which is the tube diameter of the implant as constructed. The as-cut outer diameter of the expandable implants (i.e., flow diverters and stents) described herein can be 1 mm up to about 4 mm, preferably about 2.0 mm — 3.0 mm. The expandable implants described herein can also have a crimped outer diameter or the outer diameter of the device for delivery. The expandable implants described herein can also have an expanded outer diameter, which is the outer diameter of the device following deployment. The crimped outer diameter of the expandable implants described herein can be 1.5 mm up to about 3.5 mm. The outer diameter of the device following deployment can vary depending on the anatomy or vessel size it is deployed within, in particular, for self-expanding implants. Balloon-expanded implants may also have a deployed outer diameter that is dependent upon inflation pressure of the balloon. The deployed outer diameter of the expandable implants described herein can be 2 mm up to about 5 mm.

### Endovascular Implant Delivery Systems

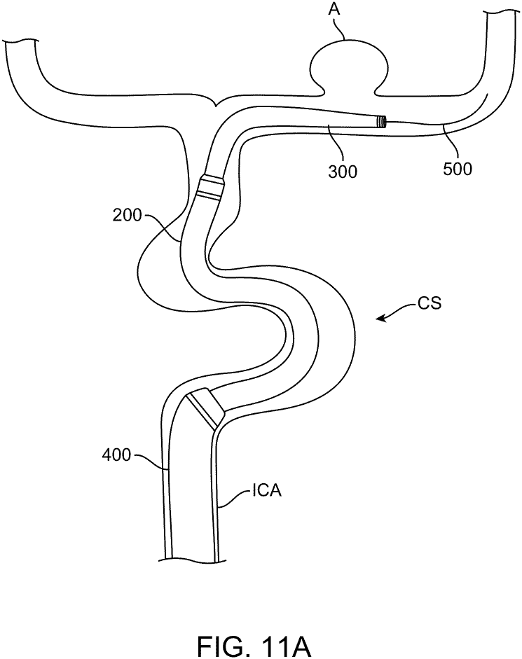

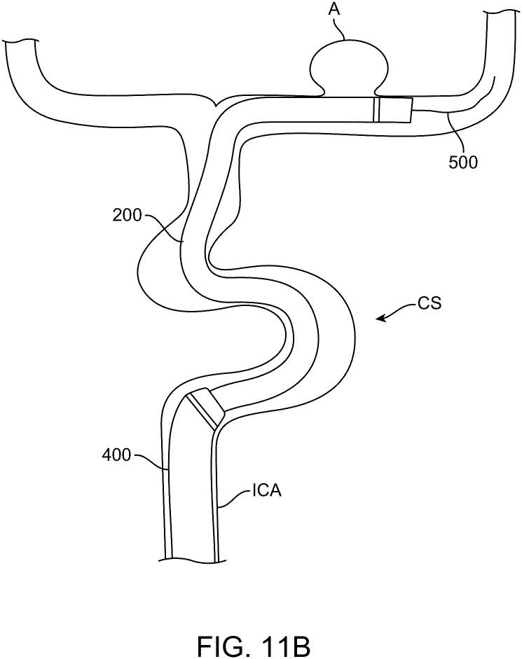

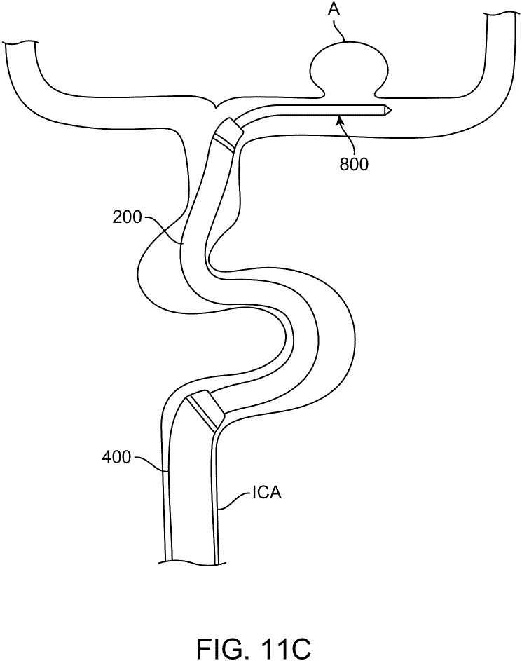

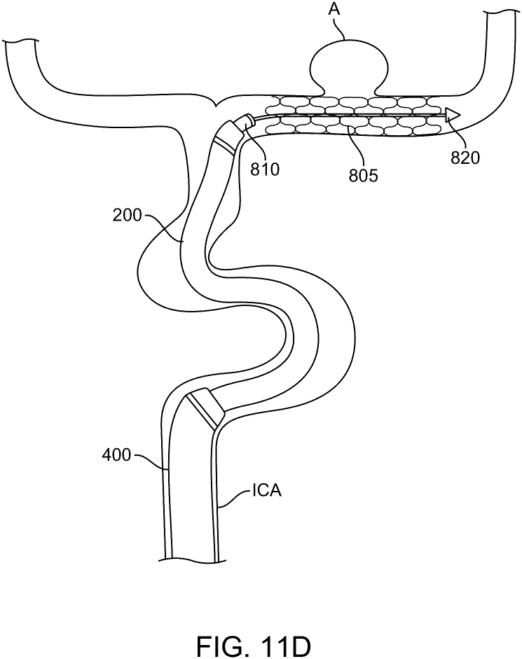

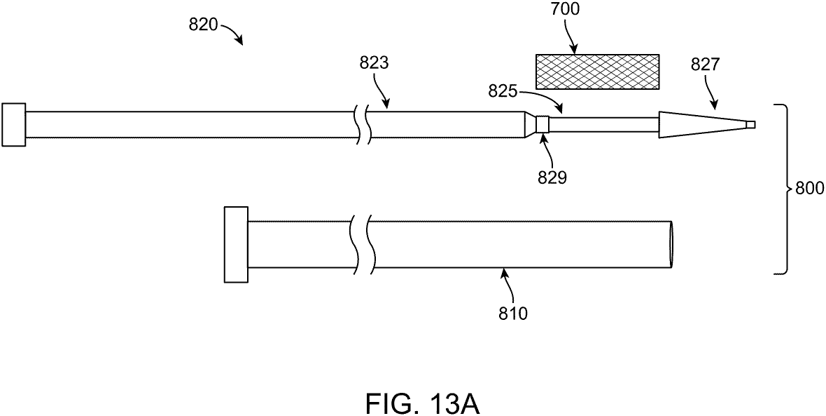

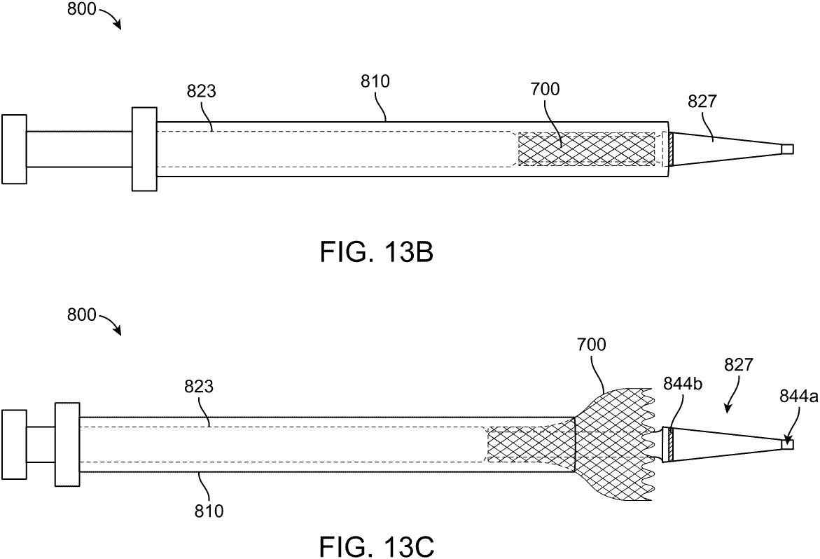

FIGS. 13A-13C illustrate a delivery system 800 that can be used for implantation of an endovascular scaffolding device 700, such as an intracranial stent or flow diverter as discussed above. The delivery system 800 can include an outer restraining sleeve 810 and an inner core member 820 having an elongate shaft 823 on which the endovascular scaffolding device can be mounted (see also FIG. 11D showing flow diverter 805 on the inner core member 520). The inner core member 820 can have an inner lumen (not shown) sized to accommodate a guidewire. The lumen can be a single, central lumen that allows the endovascular scaffolding device 700 and delivery system 800 to be delivered over a guidewire. The endovascular scaffolding device can be held in place on the inner core member 820 using bumper features and/or a recessed section and having an outer restraining sleeve 810 positioned over it. The implementation of FIGS. 13A-13C includes a shaft 823 of the inner core member 820 having a reduced diameter recessed section 825 near a distal end region that is sized to accommodate the implant in a collapsed configuration. As shown in FIG. 13B, the endovascular scaffolding device 700 is positioned in the recess 825 of the inner core member 820 and is retained in this position by the outer restraining sleeve 810. The endovascular scaffolding device 700 is held by the inner core member 820 within the recessed section 825 and deployed by expansion upon withdrawing the restraining sleeve 810 proximally. The inner core member 820 can include a grip feature 829 located at a proximal end of the recessed section 825 that is configured to prevent the endovascular scaffolding device 700 from being dragged back over shaft 823 of the inner core member 820 as the restraining sleeve 810 is withdrawn during flow diverter deployment. The grip feature 829 can be a high friction component, such as a length of thin-walled silicone or other elastomeric tube. The flow diverter 805 can be introduced in the collapsed configuration through a delivery catheter that may be initially delivered over a guidewire positioned across the aneurysm as described elsewhere herein.

The materials of the elongate shaft 823 of the inner core member 820 are selected to maintain axial integrity during deployment of the endovascular scaffolding device 700 such as flow diverter. For example, the shaft 823 and recessed section 825 can be constructed from Pebax, such as Pebax 72D. The shaft 823 and/or recessed section 825 can be braid-, coil-, or otherwise reinforced to provide axial stiffness.

The length of the outer restraining sleeve 810 is shorter than the inner core member 820 by an amount that allows the endovascular scaffolding device 700 to be fully deployed when the restraining sleeve 810 is pulled back with respect to the inner core member 820 (see FIG. 13C). The restraining sleeve 810 is configured so that it is able to be pulled back easily without dragging the endovascular scaffolding device 700 with it. For example, the restraining sleeve 810 can be constructed with multiple layers including a low friction inner liner, such as PTFE or FEP. The restraining sleeve 810 can be braid- or coil-reinforced so as not to stretch during withdrawal. The restraining sleeve 810 can also have an outer hydrophilic coating on the distal portion to improve delivery through a large-bore catheter, which will be described in more detail below.

Again with respect to FIG. 13A, the inner core member 820 can include a distal tip region 827 located distal to the recessed region 825. The distal tip region 827 of the inner core member 820 is tapered and has a flexibility, shape, taper length and taper angle configured for atraumatic delivery of the delivery system 800 to a vessel in the brain with or without a guidewire. The construction, materials, and configuration can be similar to the tapered tip 346 of catheter advancement element 300 described below with respect to access system 100 and described in U.S. Pat. No. 11,065,019 which is incorporated herein by reference in its entirety. For example, the distal tip region 827 can have at least one radiopaque taper marker configured to delineate the tapered section. The distal-most end of the inner core member 820 and a maximum outer diameter region of the taper can be identified by the taper marker 844. In some implementations, two radiopaque markers 344a, 344b identify the taper for optimum delivery purposes relative to the outer restraining sleeve 810. The outer diameter of the inner core member 820 just proximal to the taper is sized to be a smooth fit against the inner diameter of the restraining sleeve 810 so as to present a smooth leading edge to the delivery system 800 being advanced in the vasculature with or without a guidewire.

### Methods

The catheter systems described herein can be used to access and treat extracranial and intracranial arterial occlusions by providing access for working devices such as stent delivery systems. The catheters systems described herein can also be used to access and treat extracranial and cerebral aneurysms by providing access to working devices such as coil-supporting stents and flow diverters. The catheter systems described herein provide support for conventional stent delivery systems so that they may be used for intracranial stenting. The catheter systems described herein provide shorter access for the stent delivery system to navigate such that no stent delivery system is too short to reach distal intracranial vessels. The catheter systems provide monopoint manipulation at the base sheath for the various tools used in the method providing improved safety, ease of use, and single operator manipulations compared to conventional systems. The catheter systems provide easy and quick access to target sites even through tortuous anatomy to reach the target lesion.

A method for the treatment of ICAD is now described. The method can include a stent delivery system advanced over a guidewire through a catheter extending through a base sheath. The catheter can be a conventional full-length catheter, but is preferably a catheter having a larger diameter distal luminal portion 222 coupled to a smaller diameter proximal control element 230 as shown in FIGS. 1A-1B so that monopoint manipulation at the base sheath hub is possible. The base sheath (e.g. 8F) can be introduced into a blood vessel (e.g., femoral artery) and advanced to the level of at least the common carotid artery. A catheter is advanced through the hub (e.g., an RHV) on the base sheath until the distal end of the catheter exits the distal opening of the base sheath. The catheter can be advanced into the high ICA. A guidewire can be advanced through the hub on the base sheath and advanced until the guidewire is positioned across the intracranial target lesion. The catheter can be parked at a location between the distal end of the base sheath and the lesion (e.g., at or near the carotid siphon) while the guidewire is advanced to its distal location. The catheter can be advanced from its parked location over the guidewire toward the target lesion to provide support for the stent delivery system. The stent delivery system can then be advanced through the catheter over the guidewire. The stent delivery system can be advanced through the same hub on the sheath as the catheter and the guidewire as discussed elsewhere herein.

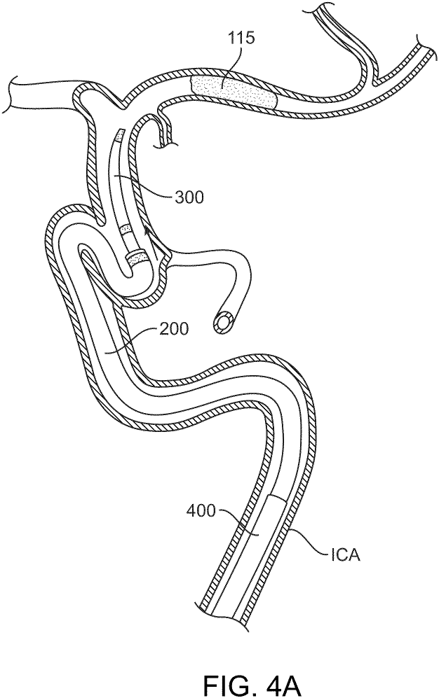

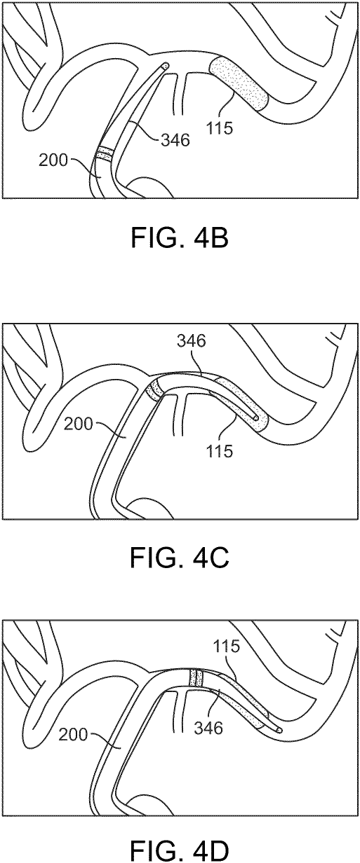

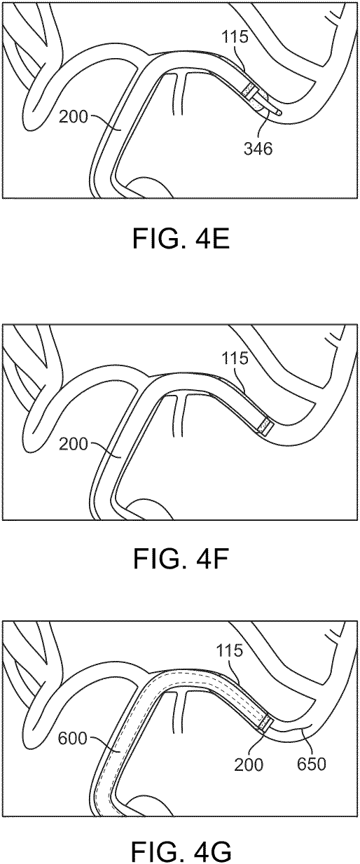

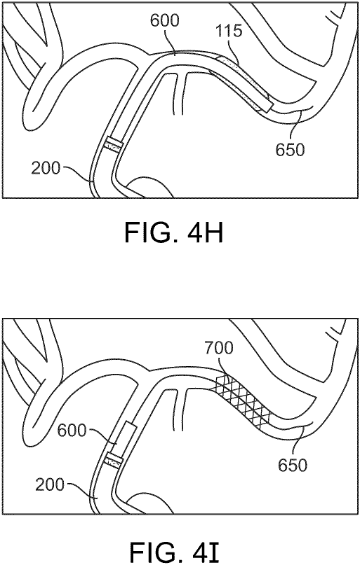

In an interrelated method, an outer catheter 200 and tapered inner catheter 300 configured to pre-dilate the lesion 115 is used prior to positioning the outer catheter 200 across the lesion 115 for unsleeving an endovascular scaffolding device 700 such as a stent. The catheter 200 having an inner catheter 300 positioned within its lumen can be inserted through the hub (e.g., RHV) on the base sheath 400 and advanced toward the lesion 115 (see FIG. 4A). A tapered end region 346 of the inner catheter 300 can be positioned distal to the distal end of the outer catheter 200 (see FIG. 4B). The catheter system of the inner and outer catheters can be advanced together until at least a portion of the tapered end region of the inner catheter is positioned at least in part over and/or beyond the lesion 115 (see FIGS. 4C-4D). The portion of the tapered end region 346 can be advanced distal to or on a downstream side of the lesion 115. The limits of a lesion 115, particularly on the downstream side, are difficult to assess for an operator. Thus, the portion of the tapered end region 346 of the inner catheter 300 need not cross the entirety of the lesion 115, but can be advanced so that it passes beyond the greatest narrowing of the lesion 115 whether or not that location is indeed distal to the entire lesion 115.

The distal end region of the outer catheter 200 can be advanced over the inner catheter 300 and positioned across the lesion 115 (see FIG. 4E). The inner catheter 300 can be withdrawn from the outer catheter 200 and the outer catheter 200 maintained in position across the lesion 115 (see FIG. 4F). The stent delivery system or microcatheter 600 can be advanced (e.g., through the hub of the outer catheter 200 or the hub of the base sheath 400 and into the distal tubular portion of the catheter 200 if the catheter 200 is a partial length catheter) to the distal end region of the outer catheter 200 (see FIG. 4G). The outer catheter 200 can be withdrawn to unsleeve the stent while the stent delivery system or microcatheter 600 is maintained in place across the lesion 115 (see FIG. 4H). The endovascular scaffolding device 700 of the stent delivery system 600 can then be deployed against the lesion 115 (see FIG. 4I). Deployment of the endovascular scaffolding device 700 against the lesion 115 can be achieved depending on the stent type (e.g., balloon expanded, self-expanding). The stent can be inserted through and out of the microcatheter or a retaining sleeve of the delivery system can be withdrawn allowing for expansion of the self-expanding stent. A balloon catheter can be used to deploy a balloon expanded stent or aid in deployment of a self-expanding stent. Because the stent delivery system 600 is delivered through the outer catheter 200, a stent delivery guidewire positioned at the treatment site is unnecessary. The stent delivery system or microcatheter 600 can be pre-loaded with the endovascular scaffolding device 700 and a stent delivery stylet 650 for deployment through the outer catheter.

The lesion 115 can be pre-dilated as described herein, either with a balloon catheter and preferably with the tapered distal end 346 of the inner catheter 300, prior to deployment of the endovascular scaffolding device 700. For example, the tapered inner catheter 300 may first be advanced completely across the lesion to pre-dilate the lesion, before advancing the catheter 200 across the stenosis. Alternately, the tapered inner catheter 300 may first be advanced singly over a pre-positioned guidewire and across the lesion to dilate the lesion as a pre-treatment. This dilation is helpful, for example, when visibility of the distal anatomy is not available due to the severity of the disease. In this scenario, it may be desirable to use the rapid exchange version of the tapered inner element 300 as in FIGS. 12A-12B so that the tapered inner element 300 can then be exchanged for the combination of tapered inner element 300 and access catheter 200 quickly and without the need for an exchange length guidewire. In either case, the pre-dilation may be done with the guidewire first crossing the lesion, as in FIG. 2C, or allowing the tapered inner catheter to cross the lesion without the guidewire or with a guidewire parked inside the lumen, as in FIG. 2B.

The catheter system can be advanced over a guidewire such as a guidewire pre-positioned across the lesion. Alternatively, the guidewire can be positioned within the inner catheter lumen during advancement such that the guidewire remains fully enclosed within the inner catheter 300 (i.e., proximal to the distal opening from the single lumen of the inner catheter). The guidewire can be positioned proximal to the tapered end region 346 of the inner catheter 300 while the tapered end region 346 is being used to advance the distal access catheter 200. The tapered end region 346 of the inner catheter 300 can be positioned distal to the distal end of the outer catheter 200 and the guidewire is fully contained within the single lumen of the inner catheter 300 and navigated through the carotid artery using the tapered end region 346 to find a passage through the occlusion in the carotid artery. The tapered end region 346 can dilate the occlusion as the catheter system is advanced towards the atherosclerotic lesion 115 in the intracranial vessel.

The inner catheter can have a structure as described elsewhere herein. For example, the inner catheter can have a length configured to extend from outside a patient’s body, through a femoral artery, and into the intracranial vessel. The inner catheter can include a hypotube as the proximal segment and an intermediate segment that is an unreinforced polymer having a first durometer. The intermediate segment being proximal of the distal tapered end region and distal to the proximal segment. The tapered end region tapers from a first outer diameter down to a second outer diameter over a length of about 0.5 cm and 4.0 cm, and preferably over a length of about 1.0 cm and 3.0 cm. The outer diameters can be size-matched to the catheter being advanced as described elsewhere herein. The first outer diameter can be about 0.48″ to about 0.080″ and the second outer diameter, which can be at the distal-most terminus of the inner catheter, can be about 0.031″ up to about 0.048″. The taper angle of the wall of the tapered end region relative to the center line of the tapered end region, in turn, can be between about 0.9 to about 1.6 degrees. As such, the second outer diameter can be about 40%, 50%, or about 65% of the first outer diameter. The material hardness of the intermediate segment can vary along its length. For example, a first segment can have a material hardness of no more than 55D and a second segment located proximal to the first segment can have a material hardness of no more than 72D. The hypotube can have an inner diameter of about 0.021″ and an outer diameter of about 0.027″ and can be covered with a lubricious polymer. The inner diameter of the single lumen can be less than 0.024″. The wall thickness of the intermediate segment and an untapered portion of the tip segment can be about 0.050 inch to about 0.065 inch. The wall thickness of the intermediate segment and the untapered portion can be constant. The inner diameter of the intermediate segment and the tapered end region can be constant. The tapered end region can taper distally over a length so that a taper angle of a wall of the tapered end region relative to a center line of the tapered end region is between 0.9 and 1.6 degrees. The tapered end region can be an unreinforced, fully polymeric region having a material hardness of no more than Shore 35D. The tapered end region can taper distally from a first outer diameter to a second outer diameter, the first outer diameter being at least 1.5 times larger than the second outer diameter. The distal opening from the single lumen can have an inner diameter that is between 0.018″ and 0.024″ to accommodate a guidewire through it. The inner catheter can include at least one radiopaque marker along its length, the marker can identify the tapered end region of the inner catheter. The tapered end region can be identified by a first radiopaque marker disposed near a first outer diameter and a second radiopaque marker disposed near a second outer diameter where the tapered end region tapers distally from the first to the second outer diameter.

The outer catheter can also have a structure as described herein. The outer catheter can be full length or can include a proximal tether element extending proximally from a point of attachment near a proximal end of a flexible distal luminal portion. The outer diameter of the proximal tether near the point of attachment can be smaller than the outer diameter of the distal luminal portion near the point of attachment. The tether can be solid or hollow. The tether can be a ribbon having square edges or a round wire or hypotube. The monopoint access means a stent delivery system advanced through the outer catheter can be inserted through a port on the hub through which the catheter system is inserted. For example, the catheter system can be inserted through a first port on the sheath hub and the stent delivery system can be inserted through a second port on the same hub of the sheath. The aspiration source can also be coupled to the hub of the base sheath. The outer catheter can be one French size smaller than the base sheath through which it is inserted and the inner catheter can be one French size smaller than the outer catheter. The outer catheter can include a reinforcement layer, for example, reinforcement that extends from a proximal end to near a distal end of the tubular segment. The inner catheter can be unreinforced along at least a portion of the length of the elongate body to the distal-most end of the tapered end region. Aspiration can be drawn by the aspiration source through the outer catheter, for example, after or while the inner catheter is withdrawn, to capture embolic material with the outer catheter. Contrast agent can be injected into the intracranial vessel through the catheter lumen to visualize the lesion being treated by angiogram. The lesion, which can be calcified with severe stenosis or restenotic, can be within an intracranial vessel that is distal to a petrous portion of the ICA such as a middle cerebral artery.

The stent deployed can be a self-expanding stent so that unsleeving the stent from the stent delivery system expands the stent against the lesion. The stent can also be balloon-mounted so that inflating a balloon expands the stent against the lesion.

In an interrelated method for the treatment of ICAD, a catheter system having a luminal portion shorter than a working length of the base sheath is used allowing monopoint access at the base sheath and the steps shown with regard to FIGS. 4A-4I performed. A base sheath (e.g. 8F) is introduced into a blood vessel and advanced to the level of at least the common carotid artery or up to the level of the cervical ICA. The distal access catheter having an inner catheter positioned within its lumen can be inserted through the RHV on the base sheath and advanced toward the lesion. The distal access catheter can be have a partial length, large bore distal luminal portion (e.g., 0.072″ - 0.088″) coupled to a proximal control element near the proximal opening from the lumen. An inner catheter can be positioned within the lumen of the distal luminal portion of the outer catheter and used to advance the outer catheter towards the lesion at the distal site. Both the outer catheter and the inner catheter can be advanced through the RHV on the base sheath such that they are manipulated in a monopoint fashion. The distal end of the outer catheter exits the distal opening of the base sheath while the proximal end region of the distal luminal portion of the outer catheter remains inside the base sheath. The tapered end region of the inner catheter is positioned distal to the distal end of the outer catheter. The catheter system can be advanced together through the base sheath towards the lesion until at least a portion of the tapered end region of the inner catheter extending distal to the distal end of the outer catheter crosses the target lesion. The distal end region of the outer catheter is advanced over the inner catheter and across the lesion. The inner catheter can be withdrawn from the outer catheter while the outer catheter is maintained in position across the lesion. A stent delivery system can be inserted through the same RHV on the base sheath as the outer catheter and advanced to the distal end region of the outer catheter. The outer catheter can be withdrawn to unsleeve the stent while maintaining the stent delivery system in place. The stent delivery system and the distal access catheter are inserted through the same location at the base sheath in monopoint fashion reducing the total length necessary for the stent delivery system to reach the ICAD lesion. The stent of the stent delivery system can then be deployed against the lesion.

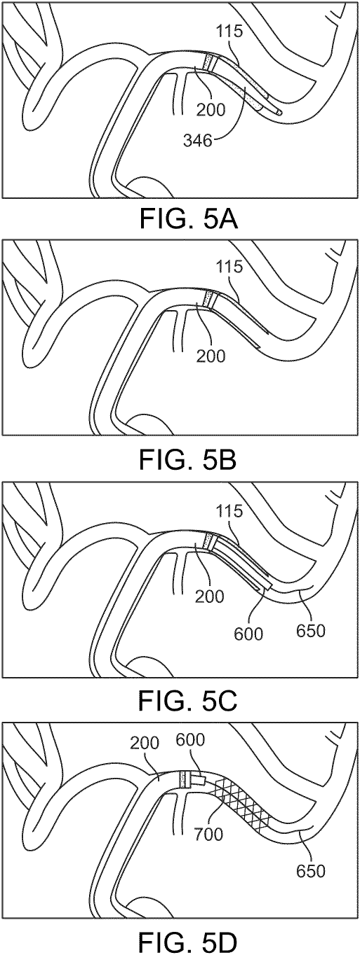

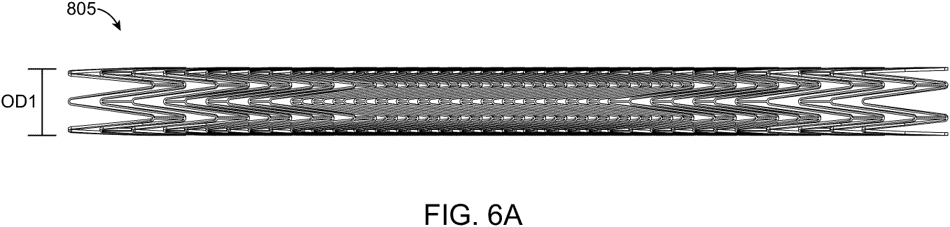

In an interrelated method of ICAD treatment, a distal access catheter 200 is delivered using an inner catheter 300. The base sheath 400 is introduced into a blood vessel from an access site and advanced to the level of the CCA or as far as the cervical ICA (see FIG. 4A). The catheter 200 having an inner catheter 300 positioned within its lumen can be inserted through the RHV of the base sheath and advanced toward the lesion 115. The tapered end region 346 of the inner catheter 300 can be positioned distal to the distal end of the outer catheter 200 and the catheter system of the inner and outer catheters can be advanced together until at least a portion of the tapered end region of the inner catheter crosses the lesion 115 (see FIGS. 4B-4D). The portion can pre-dilate the target lesion as described above. The distal end region of the outer catheter 200 can be advanced over the inner catheter 300 until the distal end is positioned just upstream to the lesion. The distal end of the catheter 200 is preferably positioned as close as possible to the lesion 115 on an upstream side near a proximal base of the lesion 115 (see FIG. 5A). The inner catheter 300 can be withdrawn after positioning of the outer catheter 200 while the position of the outer catheter 200 is maintained (see FIG. 5B). A stent delivery system or microcatheter 600 can be inserted through the outer catheter 200 and advanced out the distal end of the outer catheter 200 across the lesion 115 with or without a guidewire (see FIG. 5C). The stent delivery system 600 can be advanced into the pre-dilated lesion 115 and the endovascular scaffolding device 700 deployed against the lesion 115 (see FIG. 5D). The stent delivery system 600 can be withdrawn into the outer catheter 200 or the outer catheter 200 can be withdrawn first and the stent delivery system 600 withdrawn second. The catheter system can be advanced with a guidewire such as a guidewire pre-positioned across the lesion during crossing of the lesion with the tapered end region. Preferably, the guidewire does not extend outside the distal opening during advancement. For example, the guidewire can be positioned within the inner catheter lumen during advancement such that the guidewire remains fully enclosed within the inner catheter (i.e., proximal to the distal opening from the single lumen of the inner catheter). The guidewire can be positioned proximal to the tapered end region of the inner catheter while the tapered end region is being used to advance the outer catheter.

In preferred methods, the distal access catheter is partial length and positioned just upstream of the lesion without crossing the lesion prior to stent deployment (see, e.g., FIGS. 5A-5D). The base sheath is introduced into a blood vessel from an access site and advanced to the level of the CCA or as far as the cervical ICA. The distal access catheter having an inner catheter positioned within its lumen can be inserted through the RHV of the base sheath and advanced toward the lesion. The distal access catheter can have a partial length, large bore distal luminal portion (e.g., 0.072″ - 0.088″) coupled to a proximal control element near the proximal opening from the lumen. The inner catheter can be used to advance the outer catheter towards the lesion. Both the outer catheter and the inner catheter can be advanced through the RHV on the base sheath such that they are manipulated in a monopoint fashion. The distal end of the outer catheter exits the distal opening of the base sheath while the proximal end region of the distal luminal portion of the outer catheter remains inside the base sheath. The tapered distal end region of the inner catheter is positioned distal to the distal end of the outer catheter. The catheter system can be advanced together through the base sheath towards the lesion until at least a portion of the tapered distal end region of the inner catheter crosses the target lesion to pre-dilate the target lesion as described above. The distal end region of the outer catheter can be advanced over the inner catheter until the distal end is positioned just proximal to the lesion. The distal end of the outer catheter is preferably positioned as close as possible to the lesion. The outer catheter provides support near the base of the lesion even though the outer catheter has not crossed the lesion. In some implementations, the outer catheter can be advanced just short of the lesion or as close as possible to the lesion and then a microwire used to cross the lesion for delivery of the stent through the outer catheter. The outer catheter can be advanced to an upstream site near the lesion and parked while the microwire is advanced through the outer catheter across the lesion. The stent delivery system can then be advanced through the outer catheter over the microwire positioned across the lesion prior to stent deployment. The inner catheter can be withdrawn after positioning of the outer catheter while the position of the outer catheter is maintained. A stent delivery system can be inserted through the same RHV on the base sheath as the outer catheter and advanced through the distal luminal portion of the outer catheter and into the pre-dilated lesion. The stent delivery system and the distal access catheter are inserted through the same location at the base sheath in monopoint fashion reducing the total length necessary for the stent delivery system to reach the ICAD lesion. The stent can then be deployed against the lesion.

In interrelated method, an outer and tapered inner catheter are used to treat a cerebral aneurysm by positioning the outer catheter across an aneurysm and then used to deploy an intracranial stent or a flow diverter through a microcatheter across the aneurysm.

In each of the methods described herein, aspiration may be applied to the outer catheter through the base sheath as described in more detail elsewhere. Aspiration through the catheter system limits or prevents distal embolic particles from traveling distally during the stent placement steps.

In an interrelated method, the endovascular scaffolding device 700 may be advanced through the inner catheter 300 rather than a microcatheter or stent delivery system 600. In this method the inner catheter 300 design is configured to allow an expandable endovascular scaffolding device 700 to be advanced through the inner lumen 368 of the catheter 300. For example, the inner lumen 368 may be lined with a low friction liner such as PTFE and have a wall thickness and/or reinforcement along at least part of the length to avoid ovalization of the inner lumen 368 when it is positioned across the curvature to reach the desired anatomy. Alternately, the inner catheter 300 inner lumen 368 is configured such that it can accept a microcatheter 600, for example a microcatheter with an inner diameter of 0.021″ and outer diameter of 0.026″. Alternately the inner catheter 300 inner lumen 368 is configured to be pre-loaded with an endovascular scaffolding device 700 at or near the distal end of the inner catheter 300. If the endovascular scaffolding device 700 causes the tapered end region 346 of the inner catheter 300 to be too stiff, the endovascular scaffolding device 700 may sit further back to allow the inner catheter 300 to advance in an atraumatic fashion without a guidewire. In either of these methods, the inner catheter 300 need not be removed to advance the stent delivery system or microcatheter 600, and deploy the endovascular scaffolding device 700, thus eliminating one exchange step.

The target lesion location can vary including sites distal to the petrous portion of the ICA, including M1 and M2. The working device being delivered in the methods described herein can vary including self-expanding or balloon expanding or balloon-mounted stents or other implants configured to be left in place at the target site. The working device being delivered in the methods described herein can also be temporary implants such as stent retrievers that are configured to be temporarily positioned at the target site and then removed following a particular step in the procedure. In some methods, dilation alone is sufficient to treat the ICAD lesion and no implant deployment is performed.

The distal end of the base sheath can be advanced to a location proximal of the bifurcation of the internal and external carotid arteries within the common carotid artery. The distal end of the base sheath can also be advanced distal to the bifurcation into, for example, into a region of the internal carotid artery such as the cervical ICA.

The lesion being treated can be pre-dilated prior to stent deployment. A lesion may narrow the vessel and be so tight relative to the stent delivery catheter that pre-dilation becomes necessary prior to stent delivery. A portion of the stent delivery catheter can be used to pre-dilate the lesion in this situation, for example, a distal end region of a balloon catheter. Preferably, the lesion is pre-dilated by crossing the lesion with at least a portion of the tapered distal tip of the inner catheter. The tapered distal tip of the inner catheter can be positioned distal to the balloon of a balloon expandable stent delivery system such that the pre-dilation is performed not be the balloon catheter per se, but by the tapered distal tip extending distal to the balloon catheter. In still further implementations, a self-expandable stent may be mounted on a stent delivery catheter that has a tapered distal tip similar to the inner catheter as described elsewhere herein. The stent delivery catheter having the tapered distal tip can be advanced through the outer catheter positioned just proximal to the lesion so that the stent mounted on the stent delivery catheter (e.g., mounted proximal to the tapered region on a cylindrical portion of the stent delivery catheter) spans the lesion for deployment such as via a pin and pull type sheath over the stent delivery catheter.

The tapered distal tip provides a gradual enlargement from the very soft and small distal-most end (e.g., OD between about 0.028″ to about 0.032″) that transitions to a larger outer diameter (e.g., OD between about 0.060″ up to about 0.080″) of the outer catheter, gradually and in a fashion that is less traumatic to the vessel than, for example, inserting a balloon catheter to perform the dilation. As discussed elsewhere herein, the tapered distal tip is configured to locate the narrowed path into the lesion and enlarge the path to a greater inner diameter as it is advanced into the lesion. The narrowed path through the lesion that is enlarged by the tapered distal tip can be through the central lumen of the vessel surrounded by atherosclerotic plaque on all sides. The narrowed path through the lesion that is enlarged by the tapered distal tip may also include a region that is not surrounded on all sides by atherosclerotic plaque and can be a path between a plaque on just one side of the vessel wall and another side of the vessel wall having no plaque.

In some procedures, the lesion may not be stented once pre-dilation is performed. Meaning, the ICAD lesion may be dilated without stent delivery. In a lesion that is particularly thrombotic a surgeon may choose to forego stenting, particularly if the lesion responds well to dilation and stays open. If the lesion does not stay open, a stent may be deployed following pre-dilation as described above.

The catheter systems described herein can be advanced to the lesion (including across the lesion) without use of a guidewire. Delivering a stent through the outer catheter as described herein, instead of over a guidewire, prevents the stent from catching or traumatizing the vessel wall, particularly around the severe bends. A guidewire can be excluded entirely from the system or may be positioned in reserve, for example, parked proximal of the distal end of the inner catheter within at least a portion of the single lumen of the inner catheter. The tapered distal tip of the inner catheter can be used to cross the lesion for positioning the distal access catheter relative to the lesion while the guidewire remains fully enclosed within the inner catheter. If the distal tip is unable to cross to pre-dilate, the guidewire may be advanced distal to the distal tip and positioned across the lesion to assist in passing the distal tip through the lesion. The tapered distal tip of the inner catheter is a preferred first attempt because it is safer than the guidewire. The tapered distal tip is highly flexible and less likely to cause vessel rupture, dissection, and intimal tear the cerebral vessels during passage compared to microwires or balloon catheter as discussed elsewhere herein. The distal access catheter can cross the lesion, if desired, and depending on the lesion being treated. Withdrawal of the outer catheter unsleeving the stent delivery system can include withdrawing partially so that the catheter remains just upstream or withdrawing fully from the RHV of the base sheath.

In the methods described herein the component crossing the lesion, whether the guidewire, the distal tip of the inner catheter, and/or the distal access catheter, preferably does not cross the lesion such that it extends to the distal or downstream side of the lesion.

In some patients, the atherosclerotic disease is located within the extracranial anatomy leading to the more distal target sites within the intracranial anatomy. For example, at least a region of the carotid artery can be at least partially occluded. The extracranial occlusion can be treated prior to treating the intracranial occlusion. In some implementations, the extracranial occlusion can be treated by dilating the occlusion as the catheter advancement element navigates through the extracranial occlusion. The occlusion can then be treated using aspiration and/or delivery of a carotid stent. For example, the catheter advancement element extending distal to the extracranial occlusion can be used to advance a large-bore catheter over it so the distal end of the lumen is positioned near a proximal end of the extracranial occlusion. The catheter advancement element can be removed and aspiration drawn through the large-bore catheter to aspirate the occlusion in a proximal to distal direction. Alternatively, the catheter advancement element can be removed and a stent delivery system advanced through the large-bore catheter to deploy a stent within the occlusion. In other implementations, the extracranial occlusion can be treated by aspiration and/or stenting without pre-dilating the occlusion. And in still further implementations, the aspiration can be applied through the large-bore catheter in a distal to proximal direction. The large-bore catheter can be advanced so that the distal end of the lumen is positioned near a distal end region of the extracranial occlusion. The catheter advancement element can be removed and aspiration drawn through the large-bore catheter to aspiration the occlusion as the catheter is being withdrawn proximally. The catheter advancement element extending through the catheter can include a guidewire parked within a distal end region of the lumen so that the guidewire is available for use, if desired, but not leading the catheter system through the occlusion.

In an implementation of a method of treating atherosclerotic disease in an extracranial artery, a tapered inner catheter is used to pre-dilate the lesion prior to positioning a stent against the lesion. The tapered inner catheter can have a tubular elongate body with a single lumen extending through it and include a proximal segment, an intermediate segment, and a flexible, distal tapered end region. The intermediate segment can be an unreinforced polymer as can the distal tapered end region. The unreinforced polymer of the tapered end region can have a material hardness that is less than that of the intermediate segment. The inner catheter can extend through an outer catheter and the catheter system can be advanced through a base sheath towards the atherosclerotic lesion.

The distal end of the base sheath can be advanced from an access site (e.g., a femoral artery) to a location near the lesion. The distal end of the base sheath can be advanced to the common carotid artery proximal of a bifurcation between the internal carotid artery and an external carotid artery. The distal end of the base sheath can be advanced distal to the bifurcation within either the external carotid artery or the internal carotid artery. The positioning of the distal end of the sheath can vary depending on the location of the disease being treated. The atherosclerotic lesion can be in at least one of the common carotid artery, the external carotid artery, or the internal carotid artery.

The catheter system can be inserted through the hub (e.g., RHV) on the base sheath and advanced toward the lesion. The outer catheter can be advanced over the inner catheter and the distal end region of the outer catheter positioned across the lesion. The tapered end region of the inner catheter can be positioned distal to the distal end of the outer catheter so that at least a portion of the tapered end region of the inner catheter can cross the lesion as the catheter system is advanced. The inner catheter can be withdrawn from the outer catheter and the outer catheter maintained in place. A stent delivery system having a stent can be advanced through the catheter lumen to the distal end region of the outer catheter. The stent of the stent delivery system can be deployed against the lesion.

The outer catheter can be a standard full-length catheter or can be a spined catheter as described elsewhere herein having a proximal tether element extending proximally from a point of attachment near the proximal end of the luminal portion. The outer catheter can be advanced over the inner catheter so that the distal end region of the outer catheter is positioned across the lesion. The outer catheter can be maintained in place across the lesion as the inner catheter is withdrawn from its lumen. The outer catheter can be withdrawn after the stent delivery system is positioned to the distal end region of the outer catheter so that withdrawing the outer catheter unsleeves the stent delivery system while it is maintained in place.

Crossing the lesion with the tapered end region of the inner catheter can dilate the lesion prior to deployment of the stent. The catheter system can be advanced over a guidewire. During advancement of the tapered end region of the inner catheter through the lesion, the guidewire can be positioned within the single lumen of the inner catheter so that a distal end of the guidewire is positioned proximal to a distal opening from the single lumen and does not extend through the lesion unsheathed. In other words, the guidewire can remain parked inside the lumen near the distal end of the inner catheter and does not extend out of the distal opening of the single lumen. Alternatively, the guidewire can be positioned across the lesion prior to the tapered end region of the inner catheter crossing the lesion.

Crossing the lesion can include navigating the catheter system past the lesion while the tapered end region of the inner catheter is positioned distal to the distal end of the outer catheter and the guidewire is fully contained within the single lumen of the inner catheter. The tapered end region of the inner catheter can be used to navigate the catheter system past the lesion to find a passage through the lesion.

A method for the treatment of cerebral or intracranial aneurysm is now described. The method can include a flow diverter and delivery system advanced over a guidewire through an outer catheter extending through a base sheath. The catheter can be a conventional full-length catheter, but is preferably a catheter having a larger diameter distal luminal portion 222 coupled to a smaller diameter proximal control element 230 as shown in FIGS. 1A-1B so that monopoint manipulation at the base sheath hub is possible. The base sheath 400 can be introduced into a blood vessel (e.g., femoral artery) and advanced to the level of at least the common carotid artery towards an intracranial or cerebral vessel having a segment with an aneurysm. An outer catheter 200 is advanced through the hub (e.g., an RHV) on the base sheath 400 until the distal end of the catheter exits the distal opening of the base sheath 400 (see FIG. 11A). The catheter 200 can be advanced into the high ICA. The outer catheter 200 can be part of a catheter system including an inner catheter 300 having a tapered end region that extends distal to the distal end of the outer catheter 200. The outer catheter 200 can be navigated through the carotid siphon CS towards the aneurysm A aided by the inner catheter 300. The outer catheter 200 and inner catheter 300 can be advanced until at least a portion of the tapered end region of the inner catheter 300 is positioned across the target aneurysm as illustrated in FIG. 11A. Alternatively, a guidewire 500 can be advanced through the hub on the base sheath 400 and advanced until the guidewire 500 is positioned across the aneurysm A while the outer catheter 200 remains parked at a location between the distal end of the base sheath 400 and the aneurysm A (e.g., at or near the carotid siphon CS).

The distal end region of the outer catheter 200 can be advanced over the inner catheter 300 and positioned across the aneurysm A. The inner catheter 300 can be withdrawn from the outer catheter 200 and the outer catheter 200 maintained in position across the aneurysm (see FIG. 11B). The outer catheter 200 can have an ID of between 2.0 mm and 3.0 mm that is configured to receive an endovascular scaffolding device such as a flow diverter 805 and the delivery system 800 for the scaffolding device. The delivery system 800 and flow diverter 805 can be advanced (e.g., through the hub of the outer catheter 200 or the hub of the base sheath 400 and into the distal tubular portion of the catheter 200 if the catheter 200 is a partial length catheter) to the distal end region of the outer catheter 200. The outer catheter 200 can be withdrawn to expose the delivery system 800 while the delivery system 800 is maintained across the aneurysm A (see FIG. 11C). The flow diverter 805 of the delivery system 800 can then be deployed across the aneurysm A (see FIG. 11D).

The delivery system 800 can include an inner tubular core member or rod 820 and an outer tubular member or restraining sleeve 810. The flow diverter 805 can be mounted on the inner tubular member 820 and constrained by the outer tubular member 810 during delivery. The flow diverter constrained by the outer sleeve 810 can be deliverable through a delivery catheter having an inner diameter that is between 2.0 mm and 3.0 mm. Deployment of the flow diverter 805 across the aneurysm A can be achieved, for example, in reference to FIG. 11D, by retracting the outer sleeve 810 of delivery system 800 to expose flow diverter 805 while an inner rod 820 remains in place distal to the aneurysm A.

The flow diverter 805 can be any of those described previously. For example, the flow diverter can be a laser-cut expandable metal tube. The flow diverter can be formed of first and second expandable tubes where each is a laser cut metal tube. The first expandable tube can be a laser cut metal tube and the second expandable tube can be a braided tube. Alternatively, the first expandable tube can be a laser cut metal tube and the second expandable tube can be a polymer sleeve. The flow diverter can have a compound construction. The compound construction can include two end sections constructed from laser-cut tube and a middle section that is a braid.

### Materials

One or more components of the catheters described herein may include or be made from a variety of materials including one or more of a metal, metal alloy, polymer, a metal-polymer composite, ceramics, hydrophilic polymers, polyacrylamide, polyethers, polyamides, polyethylenes, polyurethanes, copolymers thereof, polyvinyl chloride (PVC), PEO, PEO-impregnated polyurethanes such as Hydrothane, Tecophilic polyurethane, Tecothane, PEO soft segmented polyurethane blended with Tecoflex, thermoplastic starch, PVP, and combinations thereof, and the like, or other suitable materials.

Some examples of suitable metals and metal alloys for catheter braid or coil reinforcement include stainless steel, such as 304V, 304L, and 316LV stainless steel; mild steel; nickel-titanium alloy such as linear-elastic and/or super-elastic Nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material and as described elsewhere herein.

Inner liner materials of the catheters described herein can include low friction polymers such as PTFE (polytetrafluoroethylene) or FEP (fluorinated ethylene propylene), PTFE with polyurethane layer (Tecoflex). Reinforcement layer materials of the catheters described herein can be incorporated to provide mechanical integrity for applying torque and/or to prevent flattening or kinking such as metals including stainless steel, Nitinol, Nitinol braid, helical ribbon, helical wire, cut stainless steel, or the like, or stiff polymers such as PEEK. Reinforcement fiber materials of the catheters described herein can include various high tenacity polymers like Kevlar, polyester, meta-para-aramide, PEEK, single fiber, multi-fiber bundles, high tensile strength polymers, metals, or alloys, and the like. Outer jacket materials of the catheters described herein can provide mechanical integrity and can be contracted of a variety of materials such as polyethylene, polyurethane, PEBAX, nylon, Tecothane, and the like. Other coating materials of the catheters described herein include paralene, Teflon, silicone, polyimide-polytetrafluoroetheylene, and the like. The inner liner may further include different surface finishes, such as dimples, bumps, ridges, troughs. The surface finishes may be randomly disposed, linearly disposed, spirally disposed, or otherwise disposed using a specific pattern along the length of the catheter. It is further contemplated that the inner liner may include a mixture of different surface finishes, for example, one section may have dimples, another section may have troughs, etc. Additionally, the surface finish may be incorporated along the entire length of the catheter or only in sections of the catheter. It is also contemplated that the inner liner may further include an electrosprayed layer, whereby materials could be incorporated into the inner liner. Examples of materials can include low friction materials as described above. Alternatively, the electrosprayed or electrospun layer may incorporate a beneficial agent that becomes free from the coating when exposed to blood, or to compression from a clot, for example, the beneficial agent may be a tissue plasminogen activator (tPA) or heparin encased in alginate.

Implementations describe catheters and delivery systems and methods to deliver catheters to target anatomies. However, while some implementations are described with specific regard to delivering catheters to a target vessel of a neurovascular anatomy such as a cerebral vessel, the implementations are not so limited and certain implementations may also be applicable to other uses. For example, the catheters can be adapted for delivery to different neuroanatomies, such as subclavian, vertebral, carotid vessels as well as to the coronary anatomy or peripheral vascular anatomy, to name only a few possible applications. It should also be appreciated that although the systems described herein are described as being useful for treating a particular condition or pathology, that the condition or pathology being treated may vary and are not intended to be limiting. Use of the terms “embolus,” “embolic,” “emboli,” “thrombus,” “occlusion,” “lesion”, etc. that relate to a target for treatment using the devices described herein are not intended to be limiting. The terms may be used interchangeably and can include, but are not limited to a blood clot, air bubble, small fatty deposit, or other object carried within the bloodstream to a distant site or formed at a location in a vessel. The terms may be used interchangeably herein to refer to something that can cause a partial or full occlusion of blood flow through or within the vessel.

In various implementations, description is made with reference to the figures. However, certain implementations may be practiced without one or more of these specific details, or in combination with other known methods and configurations. In the description, numerous specific details are set forth, such as specific configurations, dimensions, and processes, in order to provide a thorough understanding of the implementations. In other instances, well-known processes and manufacturing techniques have not been described in particular detail in order to not unnecessarily obscure the description. Reference throughout this specification to “one embodiment,” “an embodiment,” “one implementation, “an implementation,” or the like, means that a particular feature, structure, configuration, or characteristic described is included in at least one embodiment or implementation. Thus, the appearance of the phrase “one embodiment,” “an embodiment,” “one implementation, “an implementation,” or the like, in various places throughout this specification are not necessarily referring to the same embodiment or implementation. Furthermore, the particular features, structures, configurations, or characteristics may be combined in any suitable manner in one or more implementations.

The use of relative terms throughout the description may denote a relative position or direction. For example, “distal” may indicate a first direction away from a reference point. Similarly, “proximal” may indicate a location in a second direction opposite to the first direction. The reference point used herein may be the operator such that the terms “proximal” and “distal” are in reference to an operator using the device. A region of the device that is closer to an operator may be described herein as “proximal” and a region of the device that is further away from an operator may be described herein as “distal”. Similarly, the terms “proximal” and “distal” may also be used herein to refer to anatomical locations of a patient from the perspective of an operator or from the perspective of an entry point or along a path of insertion from the entry point of the system. As such, a location that is proximal may mean a location in the patient that is closer to an entry point of the device along a path of insertion towards a target and a location that is distal may mean a location in a patient that is further away from an entry point of the device along a path of insertion towards the target location. However, such terms are provided to establish relative frames of reference, and are not intended to limit the use or orientation of the catheters and/or delivery systems to a specific configuration described in the various implementations.

The word “about” means a range of values including the specified value, which a person of ordinary skill in the art would consider reasonably similar to the specified value. In embodiments, about means within a standard deviation using measurements generally acceptable in the art. In embodiments, about means a range extending to +/- 10% of the specified value. In embodiments, about includes the specified value.

While this specification contains many specifics, these should not be construed as limitations on the scope of what is claimed or of what may be claimed, but rather as descriptions of features specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or a variation of a sub-combination. Similarly, while operations are depicted in the drawings in a particular order, this should not be understood as requiring that such operations be performed in the particular order shown or in sequential order, or that all illustrated operations be performed, to achieve desirable results. Only a few examples and implementations are disclosed. Variations, modifications and enhancements to the described examples and implementations and other implementations may be made based on what is disclosed.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.”

Use of the term “based on,” above and in the claims is intended to mean, “based at least in part on,” such that an unrecited feature or element is also permissible.

The catheter system disclosed herein may be packaged together in a single package, where the catheters and catheter advancement element are packaged in a coil tube. The finished package would be sterilized using sterilization methods such as Ethylene oxide or radiation and labeled and boxed. Instructions for use may also be provided in-box or through an internet link printed on the label.

### P Embodiments

P Embodiment 1. A method of treating intracranial atherosclerotic disease, the method comprising: advancing a catheter system through a base sheath towards an intracranial vessel having an atherosclerotic lesion, the catheter system comprising: an inner catheter having a tubular elongate body with a single lumen and a flexible, distal tapered end region; and an outer catheter having a catheter lumen and a distal end; positioning the tapered end region of the inner catheter distal to the distal end of the outer catheter; crossing the lesion with at least a portion of the tapered end region of the inner catheter; advancing the outer catheter over the inner catheter and positioning a distal end region of the outer catheter across the lesion; withdrawing the inner catheter from the catheter lumen and maintaining the outer catheter in place across the lesion; advancing a stent delivery system comprising a stent through the catheter lumen to the distal end region of the outer catheter; withdrawing the outer catheter to unsleeve the stent and maintaining the stent delivery system in place; and deploying the stent of the stent delivery system against the lesion.

P Embodiment 2. The method of P Embodiment 1, further comprising navigating the catheter system through a carotid artery using the tapered end region of the inner catheter to find a passage through an occlusion in the carotid artery.

P Embodiment 3. The method of P Embodiment 1 or 2, wherein crossing the lesion with the at least a portion of the tapered end region of the inner catheter pre-dilates the lesion.

P Embodiment 4. The method of any one of P Embodiment 1-3, wherein advancing the catheter system comprises advancing the catheter system over a guidewire.

P Embodiment 5. The method of P Embodiment 4, wherein the guidewire is pre-positioned across the lesion.

P Embodiment 6. The method of P Embodiment 4, wherein the guidewire is positioned within the single lumen of the inner catheter proximal to a distal opening from the single lumen.

P Embodiment 7. The method of P Embodiment 6, wherein advancing a catheter system through a base sheath further comprises navigating the catheter system through a carotid artery while the tapered end region of the inner catheter is positioned distal to the distal end of the outer catheter and the guidewire is fully contained within the single lumen of the inner catheter.

P Embodiment 8. The method of P Embodiment 7, wherein navigating the catheter system through the carotid artery comprises using the tapered end region of the inner catheter to find a passage through an occlusion in the carotid artery.

P Embodiment 9. The method of P Embodiment 8, wherein the tapered end region of the inner catheter dilates the occlusion in the carotid artery as the catheter system is advanced towards the atherosclerotic lesion in the intracranial vessel.

P Embodiment 10. The method of P Embodiment 4, wherein a distal end of the guidewire is positioned proximal to the distal tapered end region of the inner catheter during the advancing step.

P Embodiment 11. The method of P Embodiment 4, wherein the guidewire is a 0.014″ to 0.024″ guidewire.

P Embodiment 12. The method of any one of P Embodiments 1-11, wherein the inner catheter has a length configured to extend from outside a patient’s body, through a femoral artery, and to the intracranial vessel.

P Embodiment 13. The method of any one of P Embodiments 1-12, wherein the inner catheter further comprises a proximal segment comprising a metal reinforced segment and an intermediate segment comprising an unreinforced polymer having a first durometer, the intermediate segment proximal of the distal tapered end region and distal to the proximal segment.

P Embodiment 14. The method of P Embodiment 13, wherein the distal tapered end region is formed of a polymer that is different from the unreinforced polymer of the intermediate segment, and where the polymer of the tapered end region has a second durometer less than the first durometer.

P Embodiment 15. The method of P Embodiment 14, wherein the tapered end region tapers distally from a first outer diameter of between 0.048″ and 0.080″ to a second outer diameter of about 0.031″ up to about 0.048″ over a length that is between 0.5 cm and 4.0 cm.

P Embodiment 16. The method of P Embodiment 15, wherein the second outer diameter is at a distal-most terminus of the inner catheter.

P Embodiment 17. The method of P Embodiment 15, wherein a taper angle of a wall of the tapered end region relative to a center line of the tapered end region is between 0.9 to 1.6 degrees.

P Embodiment 18. The method of P Embodiment 15, wherein the second outer diameter is about 50% of the first outer diameter, about 40% of the first outer diameter, or about 65% of the first outer diameter.

P Embodiment 19. The method of P Embodiment 13, wherein the intermediate segment includes a first segment having a material hardness of no more than 55D and a second segment located proximal to the first segment having a material hardness of no more than 72D.

P Embodiment 20. The method of P Embodiment 13, wherein a location of a material transition between the unreinforced polymer and the metal reinforced segment is at least about 49 cm from a distal end of the elongate body.

P Embodiment 21. The method of P Embodiment 13, wherein the metal reinforced segment has an inner diameter of about 0.021″ and an outer diameter of about 0.027″.

P Embodiment 22. The method of P Embodiment 13, wherein the metal reinforced segment is covered with a lubricious polymer.

P Embodiment 23. The method of P Embodiment 13, wherein the single lumen has an inner diameter of less than 0.024 inch.

P Embodiment 24. The method of P Embodiment 13, wherein a wall thickness of the intermediate segment and an untapered portion of the tip segment is about 0.050 inch to about 0.065 inch.

P Embodiment 25. The method of P Embodiment 24, wherein the wall thickness of the intermediate segment and the untapered portion is substantially constant.

P Embodiment 26. The method of P Embodiment 13, wherein an inner diameter of the intermediate segment and the tapered end region is substantially constant.

P Embodiment 27. The method of P Embodiment 13, wherein the metal reinforced segment is a hypotube.

P Embodiment 28. The method of P Embodiment 13, wherein the metal reinforced segment is a spine.

P Embodiment 29. The method of any one of P Embodiments 1-28, wherein the tapered end region tapers distally from a first outer diameter to a second outer diameter, wherein the first outer diameter is at least 1.5 times larger than the second outer diameter.

P Embodiment 30. The method of any one of P Embodiments 1-29, wherein a distal opening from the single lumen has an inner diameter between 0.016″ and 0.028″.

P Embodiment 31. The method of any one of P Embodiments 1-30, wherein the inner catheter comprises at least one radiopaque marker along its length.

P Embodiment 32. The method of any one of P Embodiments 1-31, further comprising at least one radiopaque marker identifying the tapered end region of the inner catheter.

P Embodiment 33. The method of any one of P Embodiments 1-32, wherein the tapered end region tapers distally from a first outer diameter to a second outer diameter, and wherein a first radiopaque marker is disposed near the first outer diameter and a second radiopaque marker is disposed near the second outer diameter.

P Embodiment 34. The method of any one of P Embodiments 1-33, wherein the outer catheter comprises a flexible distal luminal portion and a proximal tether element extending proximally from a point of attachment near a proximal end of the flexible distal luminal portion, the proximal tether element extending proximally to outside the body of the patient.

P Embodiment 35. The method of P Embodiment 25, wherein an outer diameter of a portion of the proximal tether element near the point of attachment is smaller than an outer diameter of the distal luminal portion near the point of attachment.

P Embodiment 36. The method of P Embodiment 25, wherein the proximal tether element is a solid or hollow.

P Embodiment 37. The method of P Embodiment 25, wherein the proximal tether element is a ribbon, a round wire, or a hypotube.

P Embodiment 38. The method of P Embodiment 25, wherein advancing the catheter system through the base sheath further comprises inserting the catheter system into a hub on a proximal end of the base sheath.

P Embodiment 39. The method of P Embodiment 29, wherein advancing a stent delivery system further comprises inserting the stent delivery system through the hub on the proximal end of the base sheath and into the catheter lumen.

P Embodiment 40. The method of P Embodiment 30, wherein the catheter system is inserted through a first port on the hub and the stent delivery system is inserted through a second port on the hub.

P Embodiment 41. The method of P Embodiment 29, wherein an aspiration source is coupled to the hub of the base sheath.

P Embodiment 42. The method of any one of P Embodiments 1-41, wherein the outer catheter is one French size smaller than the base sheath and the inner catheter is one French size smaller than the outer catheter.

P Embodiment 43. The method of any one of P Embodiments 1-42, wherein the outer catheter comprises a reinforcement layer and wherein the inner catheter is unreinforced along at least a portion of a length of the elongate body to a distal-most end of the tapered end region.

P Embodiment 44. The method of any one of P Embodiments 1-43, further comprising applying aspiration pressure through the catheter lumen after the inner catheter is withdrawn to capture embolic material with the outer catheter.

P Embodiment 45. The method of any one of P Embodiments 1-44, further comprising applying aspiration pressure through the catheter lumen as the inner catheter is withdrawn to capture embolic material with the outer catheter.

P Embodiment 46. The method of any one of P Embodiments 1-45, further comprising injecting contrast agent into the intracranial vessel through the catheter lumen to visualize the lesion by angiogram.

P Embodiment 47. The method of any one of P Embodiments 1-46, wherein the base sheath is positioned within a femoral, basilar, radial, ulnar, or subclavian artery.

P Embodiment 48. The method of any one of P Embodiments 1-47, wherein the intracranial vessel is distal to a petrous portion of an internal carotid artery.

P Embodiment 49. The method of any one of P Embodiments 1-48, wherein the intracranial vessel is a middle cerebral artery.

P Embodiment 50. The method of any one of P Embodiments 1-49, wherein the stent is a self-expanding stent and wherein deploying the stent comprises unsleeving the stent from the stent delivery system to expand the stent against the lesion.

P Embodiment 51. The method of any one of P Embodiments 1-50, wherein the stent is a balloon-mounted stent and wherein deploying the stent comprises inflating a balloon of the stent delivery system to expand the stent against the lesion.

P Embodiment 52. The method of any one of P Embodiments 1-51, wherein the lesion is calcified with severe stenosis.

P Embodiment 53. The method of any one of P Embodiments 1-52, wherein the lesion is restenotic.

P Embodiment 54.A method of treating intracranial atherosclerotic disease, the method comprising: advancing a catheter system through a base sheath towards an intracranial vessel having an atherosclerotic lesion, the catheter system comprising: an inner catheter having a tubular elongate body with a single lumen and a flexible, distal tapered end region; and an outer catheter having a catheter lumen and a distal end; positioning the tapered end region of the inner catheter distal to the distal end of the outer catheter; crossing the lesion with at least a portion of the tapered end region of the inner catheter to pre-dilate the lesion; positioning a distal end of the outer catheter to a proximal base of the lesion; withdrawing the inner catheter from the catheter lumen and maintaining the outer catheter in place; advancing a stent delivery system comprising a stent through the catheter lumen through the distal end of the outer catheter and into the pre-dilated lesion; and deploying the stent of the stent delivery system against the lesion.

P Embodiment 55. The method of P Embodiment 54, wherein advancing the catheter system comprises advancing the catheter system with a guidewire.

P Embodiment 56. The method of P Embodiment 55, wherein the guidewire is pre-positioned across the lesion during crossing of the lesion with the tapered end region.

P Embodiment 57. The method of P Embodiment 55, wherein the guidewire is positioned within the single lumen of the inner catheter proximal to a distal opening from the single lumen during at least a portion of the advancing step.

P Embodiment 58. The method of P Embodiment 55, wherein a distal end of the guidewire is positioned proximal to the distal tapered end region of the inner catheter during the advancing step.

P Embodiment 59. The method of P Embodiment 55, wherein the guidewire is a 0.014″ to 0.024″ guidewire.

P Embodiment 60. The method of P Embodiment 54, wherein the inner catheter has a length configured to extend from outside a patient’s body, through a femoral artery, and to the intracranial vessel.

P Embodiment 61. The method of P Embodiment 54, wherein the inner catheter further comprises a proximal segment comprising a metal reinforced segment and an intermediate segment comprising an unreinforced polymer having a first durometer, the intermediate segment proximal of the distal tapered end region and distal to the proximal segment.

P Embodiment 62. The method of P Embodiment 61, wherein the distal tapered end region is formed of a polymer that is different from the unreinforced polymer of the intermediate segment, and where the polymer of the tapered end region has a second durometer less than the first durometer.

P Embodiment 63. The method of P Embodiment 62, wherein the tapered end region tapers distally from a first outer diameter of between 0.048″ and 0.080″ to a second outer diameter of about 0.031″ up to about 0.048″ over a length that is between 0.5 cm and 4.0 cm.

P Embodiment 64. The method of P Embodiment 63, wherein the second outer diameter is at a distal-most terminus of the inner catheter.

P Embodiment 65. The method of P Embodiment 63, wherein a taper angle of a wall of the tapered end region relative to a center line of the tapered end region is between 0.9 to 1.6 degrees.

P Embodiment 66. The method of P Embodiment 63, wherein the second outer diameter is about 50% of the first outer diameter, about 40% of the first outer diameter, or about 65% of the first outer diameter.

P Embodiment 67. The method of P Embodiment 61, wherein the intermediate segment includes a first segment having a material hardness of no more than 55D and a second segment located proximal to the first segment having a material hardness of no more than 72D.

P Embodiment 68. The method of P Embodiment 61, wherein a location of a material transition between the unreinforced polymer and the metal reinforced segment is at least about 49 cm from a distal end of the elongate body.

P Embodiment 69. The method of P Embodiment 61, wherein the metal reinforced segment has an inner diameter of about 0.021″ and an outer diameter of about 0.027″.

P Embodiment 70. The method of P Embodiment 61, wherein the metal reinforced segment comprises a hypotube is covered with a lubricious polymer.

P Embodiment 71. The method of P Embodiment 61, wherein the single lumen has an inner diameter of less than 0.024 inch.

P Embodiment 72. The method of P Embodiment 61, wherein a wall thickness of the intermediate segment and an untapered portion of the tip segment is about 0.050 inch to about 0.065 inch.

P Embodiment 73. The method of P Embodiment 72, wherein the wall thickness of the intermediate segment and the untapered portion is constant.

P Embodiment 74. The method of P Embodiment 54, wherein an inner diameter of the intermediate segment and the tapered end region is constant.

P Embodiment 75. The method of P Embodiment 54, wherein the tapered end region tapers distally over a length so that a taper angle of a wall of the tapered end region relative to a center line of the tapered end region is between 0.9 and 1.6 degrees.

P Embodiment 76. The method of P Embodiment 54, wherein the tapered end region tapers distally from a first outer diameter to a second outer diameter, wherein the first outer diameter is at least 1.5 times the second outer diameter.

P Embodiment 77. The method of P Embodiment 54, wherein the tapered end region tapers distally from a first outer diameter to a second outer diameter, wherein the further comprising a first radiopaque marker disposed near the first outer diameter and a second radiopaque marker disposed near the second outer diameter.

P Embodiment 78. The method of P Embodiment 54, wherein the tapered end region is an unreinforced, fully polymeric region having a material hardness of no more than Shore 35D.

P Embodiment 79. The method of P Embodiment 54, wherein a distal opening from the single lumen has an inner diameter between 0.018″ and 0.024″.

P Embodiment 80. The method of P Embodiment 54, wherein the inner catheter comprises at least one radiopaque marker along its length.

P Embodiment 81. The method of P Embodiment 54, further comprising at least one radiopaque marker identifying the tapered end region of the inner catheter.

P Embodiment 82. The method of P Embodiment 54, wherein the outer catheter comprises a flexible distal luminal portion and a proximal tether element extending proximally from a point of attachment near a proximal end of the flexible distal luminal portion, the proximal tether element extending proximally to outside the body of the patient.

P Embodiment 83. The method of P Embodiment 82, wherein an outer diameter of a portion of the proximal tether element near the point of attachment is smaller than an outer diameter of the distal luminal portion near the point of attachment.

P Embodiment 84. The method of P Embodiment 82, wherein the proximal tether element is a solid or hollow.

P Embodiment 85. The method of P Embodiment 82, wherein the proximal tether element is a ribbon, a round wire, or a hypotube.

P Embodiment 86. The method of P Embodiment 82, wherein advancing the catheter system through the base sheath further comprises inserting the catheter system into a hub on a proximal end of the base sheath.

P Embodiment 87. The method of P Embodiment 86, wherein advancing a stent delivery system further comprises inserting the stent delivery system through the hub on the proximal end of the base sheath and insert the catheter lumen.

P Embodiment 88. The method of P Embodiment 87, wherein the catheter system is inserted through a first port on the hub and the stent delivery system is inserted through a second port on the hub.

P Embodiment 89. The method of P Embodiment 86, wherein an aspiration source is coupled to the hub of the base sheath.

P Embodiment 90. The method of P Embodiment 54, wherein the outer catheter is one French size smaller than the base sheath and the inner catheter is one French size smaller than the outer catheter.

P Embodiment 91. The method of P Embodiment 54, wherein the outer catheter comprises a reinforcement layer and wherein the inner catheter is unreinforced along at least a portion of a length of the elongate body to a distal-most end of the tapered end region.

P Embodiment 92. The method of P Embodiment 54, further comprising applying aspiration pressure through the catheter lumen after the inner catheter is withdrawn to capture embolic material with the outer catheter.

P Embodiment 93. The method of P Embodiment 54, further comprising applying aspiration pressure through the catheter lumen as the inner catheter is withdrawn to capture embolic material with the outer catheter.

P Embodiment 94. The method of P Embodiment 54, further comprising injecting contrast agent into the intracranial vessel through the catheter lumen to visualize the lesion by angiogram.

P Embodiment 95. The method of P Embodiment 54, wherein the base sheath is positioned within a femoral, basilar, radial, ulnar, or subclavian artery.

P Embodiment 96. The method of P Embodiment 54, wherein the intracranial vessel is distal to a petrous portion of an internal carotid artery.

P Embodiment 97. The method of P Embodiment 54, wherein the intracranial vessel is a middle cerebral artery.

P Embodiment 98. The method of P Embodiment 54, wherein the stent is a self-expanding stent and wherein deploying the stent comprises unsleeving the stent from the stent delivery system to expand the stent against the lesion.

P Embodiment 99. The method of P Embodiment 54, wherein the stent is a balloon-mounted stent and wherein deploying the stent comprises inflating a balloon of the stent delivery system to expand the stent against the lesion.

P Embodiment 100. The method of P Embodiment 54, wherein the lesion is calcified with severe stenosis or wherein the lesion is restenotic.

P Embodiment 101. A method of treating atherosclerotic disease, the method comprising: advancing a distal end of a base sheath from a femoral artery to a common carotid artery; advancing a catheter system through the base sheath towards an atherosclerotic lesion in at least one of a common carotid artery, an external carotid artery, or an internal carotid artery, the catheter system comprising: an inner catheter having a tubular elongate body with a single lumen and a flexible, distal tapered end region; and an outer catheter comprising: a flexible, distal luminal portion having a catheter lumen extending between a distal end and a proximal end of the flexible, distal luminal portion; a proximal tether element extending proximally from a point of attachment near the proximal end of the flexible distal luminal portion to outside the body of the patient, wherein an outer diameter of a portion of the proximal tether element near the point of attachment is smaller than an outer diameter of the distal luminal portion near the point of attachment; positioning the tapered end region of the inner catheter distal to the distal end of the outer catheter; crossing the lesion with at least a portion of the tapered end region of the inner catheter; withdrawing the inner catheter from the catheter lumen and maintaining the outer catheter in place; advancing a stent delivery system comprising a stent through the catheter lumen to the distal end region of the outer catheter; and deploying the stent of the stent delivery system against the lesion.

P Embodiment 102. The method of P Embodiment 101, wherein crossing the lesion with the at least a portion of the tapered end region of the inner catheter dilates the lesion.

P Embodiment 103. The method of P Embodiment 101, wherein advancing the catheter system comprises advancing the catheter system with a guidewire positioned within the single lumen of the inner catheter so a distal end of the guidewire is positioned proximal to a distal opening from the single lumen.

P Embodiment 104. The method of P Embodiment 103, wherein crossing the lesion comprises navigating the catheter system past the lesion while the tapered end region of the inner catheter is positioned distal to the distal end of the outer catheter and without the guidewire extending out of the distal opening of the single lumen of the inner catheter.

P Embodiment 105. The method of P Embodiment 104, wherein navigating the catheter system past the lesion comprises using the tapered end region of the inner catheter to find a passage through the lesion.

P Embodiment 106. The method of P Embodiment 101, wherein the distal end of the base sheath is advanced to a location proximal of a bifurcation between the internal carotid artery and the external carotid artery.

P Embodiment 107. The method of P Embodiment 101, further comprising advancing the outer catheter over the inner catheter and positioning a distal end region of the outer catheter across the lesion.

P Embodiment 108. The method of P Embodiment 107, further comprising withdrawing the outer catheter after advancing the stent delivery system to unsleeve the stent while maintaining the stent delivery system in place.

P Embodiment 109. A method of treating atherosclerotic disease, the method comprising: advancing a distal end of a base sheath from a femoral artery to a common carotid artery; advancing a catheter system through the base sheath towards an atherosclerotic lesion in at least one of a common carotid artery, an external carotid artery, or an internal carotid artery, the catheter system comprising: an inner catheter having a tubular elongate body with a single lumen, the inner catheter comprising a proximal segment, an intermediate segment, and a flexible, distal tapered end region comprising an unreinforced polymer having a material hardness less than that of the intermediate segment, a taper length of the tapered end region being between about 0.5 cm and about 4.0 cm; and an outer catheter having a catheter lumen extending between a distal end and a proximal end; positioning the tapered end region of the inner catheter distal to the distal end of the outer catheter; crossing the lesion with at least a portion of the tapered end region of the inner catheter; withdrawing the inner catheter from the catheter lumen and maintaining the outer catheter in place; advancing a stent delivery system comprising a stent through the catheter lumen to the distal end region of the outer catheter; and deploying the stent of the stent delivery system against the lesion.

P Embodiment 110. The method of P Embodiment 109, wherein crossing the lesion with the at least a portion of the tapered end region of the inner catheter dilates the lesion.

P Embodiment 111. The method of P Embodiment 109, wherein advancing the catheter system comprises advancing the catheter system with a guidewire positioned within the single lumen of the inner catheter so a distal end of the guidewire is positioned proximal to a distal opening from the single lumen.

P Embodiment 112. The method of P Embodiment 111, wherein crossing the lesion comprises navigating the catheter system past the lesion while the tapered end region of the inner catheter is positioned distal to the distal end of the outer catheter and without the guidewire extending out of the distal opening of the single lumen of the inner catheter.

P Embodiment 113. The method of P Embodiment 112, wherein navigating the catheter system past the lesion comprises using the tapered end region of the inner catheter to find a passage through the lesion.

P Embodiment 114. The method of P Embodiment 109, wherein the distal end of the base sheath is advanced to a location proximal of a bifurcation between the internal carotid artery and the external carotid artery.

P Embodiment 115. The method of P Embodiment 109, further comprising advancing the outer catheter over the inner catheter and positioning a distal end region of the outer catheter across the lesion.

P Embodiment 116. The method of P Embodiment 115, further comprising withdrawing the outer catheter after advancing the stent delivery system to unsleeve the stent while maintaining the stent delivery system in place.

P Embodiment 117. A method of treating intracranial or cerebral aneurysm, the method comprising: advancing a catheter system through a base sheath towards an intracranial or cerebral vessel having a segment with an aneurysm, the catheter system comprising: an inner catheter having a tubular elongate body with a single lumen and a flexible, distal tapered end region; and an outer catheter having a catheter lumen and a distal end; positioning the tapered end region of the inner catheter distal to the distal end of the outer catheter; crossing the segment of vessel with the aneurysm with at least a portion of the tapered end region of the inner catheter; advancing the outer catheter over the inner catheter and positioning a distal end region of the outer catheter across the lesion; withdrawing the inner catheter from the catheter lumen and maintaining the outer catheter in place across the aneurysm; advancing a flow diverter delivery system comprising a flow diverter through the catheter lumen to the distal end region of the outer catheter; withdrawing the outer catheter while maintaining the flow diverter delivery system in place; and deploying the flow diverter across the segment with the aneurysm.

P Embodiment 118. A flow diverter system comprising: a delivery system comprising: an inner tubular member; and an outer tubular member; a flow diverter mounted on the inner tubular member and constrained by the outer tubular member during delivery; and an outer catheter having an inner diameter of between 2.0 mm and 3.0 mm configured to receive the flow diverter constrained by the outer tubular member for delivery.

P Embodiment 119. A flow diverter system as in P Embodiment 118, wherein the flow diverter is a laser-cut expandable metal tube.

P Embodiment 120. A flow diverter system as in P Embodiment 118, wherein the flow diverter is formed of first and second expandable tubes.

P Embodiment 121. A flow diverter system as in P Embodiment 120, wherein the first and second expandable tubes are each a laser cut metal tube.

P Embodiment 122. A flow diverter system as in P Embodiment 120, wherein the first expandable tube is a laser cut metal tube and the second expandable tube is a braided tube.

P Embodiment 123. A flow diverter system as in P Embodiment 120, wherein the first expandable tube is a laser cut metal tube and the second expandable tube is a polymer sleeve.

P Embodiment 124. A flow diverter system as in P Embodiment 118, wherein the flow diverter has a compound construction.

P Embodiment 125. A flow diverter system as in P Embodiment 124, wherein the compound construction comprises two end sections constructed from laser-cut tube and a middle section comprising a braid.

P Embodiment 126. A flow diverter system comprising: a flow diverter delivery system having an inner tubular member and an introducer; a flow diverter mounted on the inner tubular member and constrained by the introducer, wherein the flow diverter constrained by the introducer is deliverable through a delivery catheter having an inner diameter of between 2.0 mm and 3.0 mm.

What is claimed is:

1. A method of treating intracranial atherosclerotic disease, the method comprising:
advancing a catheter system through a base sheath towards an intracranial vessel having an atherosclerotic lesion, the catheter system comprising:
an inner catheter having a tubular elongate body with a single lumen and a flexible, distal tapered end region; and
an outer catheter having a catheter lumen and a distal end;
positioning the tapered end region of the inner catheter distal to the distal end of the outer catheter;
crossing the lesion with at least a portion of the tapered end region of the inner catheter;
advancing the outer catheter over the inner catheter and positioning a distal end region of the outer catheter across the lesion;
withdrawing the inner catheter from the catheter lumen and maintaining the outer catheter in place across the lesion;
advancing a stent delivery system comprising a stent through the catheter lumen to the distal end region of the outer catheter;
withdrawing the outer catheter to unsleeve the stent and maintaining the stent delivery system in place; and
deploying the stent of the stent delivery system against the lesion.

2. The method of claim 1, further comprising navigating the catheter system through a carotid artery using the tapered end region of the inner catheter to find a passage through an occlusion in the carotid artery.

3. The method of claim 1, wherein crossing the lesion with the at least a portion of the tapered end region of the inner catheter pre-dilates the lesion.

4. The method of claim 1, wherein advancing the catheter system comprises advancing the catheter system over a guidewire.

5. The method of claim 4, wherein the guidewire is pre-positioned across the lesion.

6. The method of claim 4, wherein the guidewire is positioned within the single lumen of the inner catheter proximal to a distal opening from the single lumen.

7. The method of claim 6, wherein advancing a catheter system through a base sheath further comprises navigating the catheter system through a carotid artery while the tapered end region of the inner catheter is positioned distal to the distal end of the outer catheter and the guidewire is fully contained within the single lumen of the inner catheter.

8. The method of claim 7, wherein navigating the catheter system through the carotid artery comprises using the tapered end region of the inner catheter to find a passage through an occlusion in the carotid artery.

9. The method of claim 8, wherein the tapered end region of the inner catheter dilates the occlusion in the carotid artery as the catheter system is advanced towards the atherosclerotic lesion in the intracranial vessel.

10. The method of claim 4, wherein a distal end of the guidewire is positioned proximal to the distal tapered end region of the inner catheter during the advancing step.

11. The method of claim 4, wherein the guidewire is a 0.014″ to 0.024″ guidewire.

12. The method of claim 1, wherein the inner catheter has a length configured to extend from outside a patient’s body, through a femoral artery, and to the intracranial vessel.

13. The method of claim 1, wherein the inner catheter further comprises a proximal segment comprising a metal reinforced segment and an intermediate segment comprising an unreinforced polymer having a first durometer, the intermediate segment proximal of the distal tapered end region and distal to the proximal segment.

14. The method of claim 13, wherein the distal tapered end region is formed of a polymer that is different from the unreinforced polymer of the intermediate segment, and where the polymer of the tapered end region has a second durometer less than the first durometer.

15. The method of claim 14, wherein the tapered end region tapers distally from a first outer diameter of between 0.048″ and 0.080″ to a second outer diameter of about 0.031″ up to about 0.048″ over a length that is between 0.5 cm and 4.0 cm.

16. The method of claim 15, wherein the second outer diameter is at a distal-most terminus of the inner catheter.

17. The method of claim 15, wherein a taper angle of a wall of the tapered end region relative to a center line of the tapered end region is between 0.9 to 1.6 degrees.

18. The method of claim 15, wherein the second outer diameter is about 50% of the first outer diameter, about 40% of the first outer diameter, or about 65% of the first outer diameter.

19. The method of claim 13, wherein the intermediate segment includes a first segment having a material hardness of no more than 55D and a second segment located proximal to the first segment having a material hardness of no more than 72D.

20. The method of claim 13, wherein a location of a material transition between the unreinforced polymer and the metal reinforced segment is at least about 49 cm from a distal end of the elongate body.

21. A method of treating intracranial atherosclerotic disease, the method comprising:
advancing a catheter system through a base sheath towards an intracranial vessel having an atherosclerotic lesion, the catheter system comprising:
an inner catheter having a tubular elongate body with a single lumen and a flexible, distal tapered end region; and
an outer catheter having a catheter lumen and a distal end;
positioning the tapered end region of the inner catheter distal to the distal end of the outer catheter;
crossing the lesion with at least a portion of the tapered end region of the inner catheter to pre-dilate the lesion;
positioning a distal end of the outer catheter to a proximal base of the lesion;
withdrawing the inner catheter from the catheter lumen and maintaining the outer catheter in place;
advancing a stent delivery system comprising a stent through the catheter lumen through the distal end of the outer catheter and into the pre-dilated lesion; and
deploying the stent of the stent delivery system against the lesion.

22. A method of treating atherosclerotic disease, the method comprising:
advancing a distal end of a base sheath from a femoral artery to a common carotid artery;
advancing a catheter system through the base sheath towards an atherosclerotic lesion in at least one of a common carotid artery, an external carotid artery, or an internal carotid artery, the catheter system comprising:
an inner catheter having a tubular elongate body with a single lumen and a flexible, distal tapered end region; and
an outer catheter comprising:
a flexible, distal luminal portion having a catheter lumen extending between a distal end and a proximal end of the flexible, distal luminal portion;
a proximal tether element extending proximally from a point of attachment near the proximal end of the flexible distal luminal portion to outside the body of the patient, wherein an outer diameter of a portion of the proximal tether element near the point of attachment is smaller than an outer diameter of the distal luminal portion near the point of attachment;
positioning the tapered end region of the inner catheter distal to the distal end of the outer catheter;
crossing the lesion with at least a portion of the tapered end region of the inner catheter;
withdrawing the inner catheter from the catheter lumen and maintaining the outer catheter in place;
advancing a stent delivery system comprising a stent through the catheter lumen to the distal end region of the outer catheter; and
deploying the stent of the stent delivery system against the lesion.

23. The method of claim 22, wherein crossing the lesion with the at least a portion of the tapered end region of the inner catheter dilates the lesion.

24. The method of claim 22, wherein advancing the catheter system comprises advancing the catheter system with a guidewire positioned within the single lumen of the inner catheter so a distal end of the guidewire is positioned proximal to a distal opening from the single lumen.

25. The method of claim 24, wherein crossing the lesion comprises navigating the catheter system past the lesion while the tapered end region of the inner catheter is positioned distal to the distal end of the outer catheter and without the guidewire extending out of the distal opening of the single lumen of the inner catheter.

26. The method of claim 25, wherein navigating the catheter system past the lesion comprises using the tapered end region of the inner catheter to find a passage through the lesion.

27. The method of claim 22, wherein the distal end of the base sheath is advanced to a location proximal of a bifurcation between the internal carotid artery and the external carotid artery.

28. The method of claim 22, further comprising advancing the outer catheter over the inner catheter and positioning a distal end region of the outer catheter across the lesion.

29. The method of claim 28, further comprising withdrawing the outer catheter after advancing the stent delivery system to unsleeve the stent while maintaining the stent delivery system in place.

30. A method of treating atherosclerotic disease, the method comprising:
advancing a distal end of a base sheath from a femoral artery to a common carotid artery;
advancing a catheter system through the base sheath towards an atherosclerotic lesion in at least one of a common carotid artery, an external carotid artery, or an internal carotid artery, the catheter system comprising:
an inner catheter having a tubular elongate body with a single lumen, the inner catheter comprising a proximal segment, an intermediate segment, and a flexible, distal tapered end region comprising an unreinforced polymer having a material hardness less than that of the intermediate segment, a taper length of the tapered end region being between about 0.5 cm and about 4.0 cm; and
an outer catheter having a catheter lumen extending between a distal end and a proximal end;
positioning the tapered end region of the inner catheter distal to the distal end of the outer catheter;
crossing the lesion with at least a portion of the tapered end region of the inner catheter;
withdrawing the inner catheter from the catheter lumen and maintaining the outer catheter in place;
advancing a stent delivery system comprising a stent through the catheter lumen to the distal end region of the outer catheter; and
deploying the stent of the stent delivery system against the lesion.

31. The method of claim 30, wherein crossing the lesion with the at least a portion of the tapered end region of the inner catheter dilates the lesion.

32. The method of claim 30, wherein advancing the catheter system comprises advancing the catheter system with a guidewire positioned within the single lumen of the inner catheter so a distal end of the guidewire is positioned proximal to a distal opening from the single lumen.

33. The method of claim 32, wherein crossing the lesion comprises navigating the catheter system past the lesion while the tapered end region of the inner catheter is positioned distal to the distal end of the outer catheter and without the guidewire extending out of the distal opening of the single lumen of the inner catheter.

34. The method of claim 33, wherein navigating the catheter system past the lesion comprises using the tapered end region of the inner catheter to find a passage through the lesion.

35. The method of claim 30, wherein the distal end of the base sheath is advanced to a location proximal of a bifurcation between the internal carotid artery and the external carotid artery.

36. The method of claim 30, further comprising advancing the outer catheter over the inner catheter and positioning a distal end region of the outer catheter across the lesion.

37. The method of claim 36, further comprising withdrawing the outer catheter after advancing the stent delivery system to unsleeve the stent while maintaining the stent delivery system in place.

38. A flow diverter system comprising:
a delivery system comprising:
an inner tubular member; and
an outer tubular member;
a flow diverter mounted on the inner tubular member and constrained by the outer tubular member during delivery; and
an outer catheter having an inner diameter of between 2.0 mm and 3.0 mm configured to receive the flow diverter constrained by the outer tubular member for delivery.

39. A flow diverter system as in claim 38, wherein the flow diverter is a laser-cut expandable metal tube.

40. A flow diverter system as in claim 38, wherein the flow diverter is formed of first and second expandable tubes.

41. A flow diverter system as in claim 40, wherein the first and second expandable tubes are laser-cut metal tubes.

42. A flow diverter system as in claim 40, wherein the first expandable tube is a laser-cut metal tube and the second expandable tube is a braided tube.

43. A flow diverter system as in claim 40, wherein the first expandable tube is a laser-cut metal tube and the second expandable tube is a polymer sleeve.

44. A flow diverter system as in claim 38, wherein the flow diverter has a compound construction.

45. A flow diverter system as in claim 44, wherein the compound construction comprises two end sections constructed from laser-cut tube and a middle section comprising a braid.

46. A flow diverter system comprising:
a flow diverter delivery system having an inner tubular member and an introducer; and
a flow diverter mounted on the inner tubular member and constrained by the introducer, wherein the flow diverter constrained by the introducer is deliverable through a delivery catheter having an inner diameter of between 2.0 mm and 3.0 mm.
